(19) United States
(12) Patent Application Publication
(10) Pub. No.: US 2008/0044860 A1
(43) Pub. Date: Feb. 21, 2008

(54) Polyketides and Their Synthesis

(76) Inventors: Sabine Gaisser, Little Chesterford (GB); Stephen Frederick Haydock, Cambridge (GB); Peter Francis Leadlay, Little Chesterford (GB); Hamish Alastair Irvine Mcarthur, New York, NY (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/580,872

(22) PCT Filed: Nov. 29, 2004
(86) PCT No.: PCT/GB04/05001

§ 371 Date: Jun. 11, 2007

(30) Foreign Application Priority Data
Nov. 28, 2003 (GB) 0327721.7

(51) Int. Cl. C07G 11/00; C07G 3/00; C07H 17/08; C12N 1/21

(52) U.S. Cl. 435/71.3; 435/71.2; 536/4.1; 536/7.2

(57) ABSTRACT

Macrolides particularly erythromycins and azithromycins, having O-mycaminosyl or O-angolosaminyl groups, particularly at the 5-position, are produced using a gene cassette comprising a combination of genes which, in an appropriate strain background, are able to direct the synthesis of mycaminose or angolosamine and to direct its subsequent transfer to an aglycone or pseudoaglycone. Synthetic genes may comprise one or more of angMIII, angMI, angB, angAI, angAII, angorf14, angorf4, tylMIII, tylMI, tylB, tylAI, tylAII, eryCVI, spnO, eryBVI, eryK, tyl Ia and ery G. Glycosyltransfer genes may comprise one or more of eryCIII, tylMII, angMII, desVII, eryBV, spnP and midI.

### FIELD OF INVENTION

The present invention relates to processes and materials (including recombinant strains) for the preparation and isolation of macrolide compounds, particularly compounds differing from natural compounds at least in terms of glycosylation. It is particularly concerned with erythromycin and azithromycin analogues wherein the natural sugar at the 5-position has been replaced. The invention includes the use of recombinant cells in which gene cassettes are expressed to generate novel macrolide antibiotics.

### BACKGROUND TO THE INVENTION

The biosynthetic pathways to the macrolide antibiotics produced by actinomycete bacteria generally involve the assembly of an aglycone structure, followed by specific modifications which may include any or all of: hydroxylation or other oxidative steps, methylation and glycosylation. In the case of the 14-membered macrolide erythromycin A, these modifications consist of the specific hydroxylation of 6-deoxyerythronolide B to erythronolide B which is catalysed by EryF, followed by the sequential attachment of dTDP-L mycarose via the hydroxyl group at C-3 catalysed by the mycarosyltransferase EryBV (Staunton and Wilkinson, 1997). The attachment of dTDP-D-desosamine via the hydroxyl group at C-5, catalysed by EryCIII, then results in the production of erythromycin D, the first intermediate with antibiotic activity. Erythromycin D is subsequently converted to erythromycin A by hydroxylation at C-12 (EryK) and O-methylation (EryG) on the mycarosyl group, this order being preferred (Staunton and Wilkinson, 1997). The biosynthesis of dTDP-L-mycarose and dTDP-D-desosamine has been studied in detail (Gaisser et al., 1997; Summers et al., 1997; Gaisser et al., 1998; Salah-Bey et al., 1998).

Recently, a 3.1 Å high-resolution X-ray investigation of the interaction of ribosomes with macrolides (Schlunzen et al., 2001, Hansen et al., 2002) has revealed key interactions giving direct insights into ways in which macrolide templates might be adapted, by chemical or biological approaches, for increased ribosomal binding and inhibition and for improved effectiveness against resistant organisms. In particular, previous indications about the importance of the sugar substituent at the C-5 hydroxyl of the macrocycle for ribosomal binding were fully borne out by the structural analysis. This substituent extends towards the peptidyl transferase centre and in the case of 16-membered macrolides, which bear a disaccharide at C-5, reaches further into the peptidyl transferase centre, thus providing a molecular basis for the observation that 16-membered macrolides inhibit ribosomal capacity to form even a single peptide bond (Poulsen et al., 2000). This suggests that erythromycins with alternative substituents at the C-5 positions, for example mycaminosyl and angolosaminyl erythromycins, and in particular mycaminosyl and 4′-O substituted mycaminosyl erythromycins, are highly desirable as potential anti-bacterial agents.

Since post-polyketide synthase modifications are often critical for biological activity (Liu and Thorson, 1994; Kaneko et al., 2000), there has been increasing interest in understanding the mechanism and specificity of the enzymes involved to engineer the biosynthesis of diverse novel hybrid macrolides with potentially improved activities. Recent work has demonstrated that the manipulation of sugar biosynthetic genes is a powerful approach to isolate novel macrolide antibiotics. The recently demonstrated relaxed specificity of the glycosyltransferases is crucial for this approach (see Méndez and Salas, 2001 and references therein). In the pathways to erythromycin A and methymycin/neomethymycin, the production of hybrid macrolides has been observed after inactivation of specific genes involved in the biosynthesis of deoxyhexoses (Gaisser et al., 1997; Summers et al., 1997; Gaisser et al., 1998; Salah-Bey et al., 1998; Zhao et al., 1998a; Zhao et al., 1998b) or after the expression of genes from different biosynthetic gene clusters (Zhao et al., 1999). A relaxed specificity towards the sugar substrate has also been reported for glycosyltransferases that have been expressed in heterologous strains, including glycosyltransferases from the pathways to vancomycin (Solenberg et al., 1997), elloramycin (Wohlert et al., 1998), oleandomycin (Doumith et al., 1999; Gaisser et al., 2000), pikromycin (Tang and McDaniel, 2001), epirubicin (Madduri et al., 1998), avermectin (Wohlert et al., 2001) and spinosyn (Gaisser et al., 2002a). Most of the successful alterations so far reported have involved relaxed specificity towards the activated sugar moiety, while as yet only isolated examples are known where a glycosyltransferase targets its deoxysugar to an alternative aglycone substrate (Spagnoli et al., 1983; Trefzer et al., 1999). Both WO 97/23630 and WO 99/05283 describe the production of erythromycins with an altered glycosylation pattern in culture supernatants by deletion of a specific sugar biosynthesis gene. Thus WO 99/05283 describes low but detectable levels of 5-O-dedesosaminyl-5-O-mycaminosyl erythromycin D in the culture supernatant of an eryCIV knockout strain of S. erythraea. It also has been demonstrated that the use of the gene cassette technology described in patent WO01/79520 is a powerful and potentially general approach to isolate novel macrolide antibiotics by expressing combinations of genes in mutant strains of S. erythraea (Gaisser et al., 2002b). WO01/79520 also describes the detection of 5-O-dedesosaminyl-5-O-mycaminosyl erythromycin A in culture supernatants of the S. erythraea strains SGQ2pSGCIII and SGQ2p(mycaminose)CIII, fed with 3-O-mycarosyl erythronolide B. However, the low levels of 5-O-dedesosaminyl-5-O-mycaminosyl erythromycin A make this a less than optimal method for producing this valuable material on large scales and similar problems were encountered synthesizing 5-O-dedesosaminyl-5-O-mycaminosyl erythromycin A using chemical methods (Jones et al., 1969). EP 1024145 refers to the isolation of azithromycin analogues carrying a mycaminosyl residue such as 5-O-dedesosaminyl-5-O-mycaminosyl azithromycin and 3″-desmethyl-5-O-dedesosaminyl-5-O-inycaininosyl azithromycin. However the only examples given in this area are “prophetic examples” and there is no evidence that they could actually be put into practice.

Therefore, the present invention provides the first demonstration of an efficient and highly effective method for making significant quantities of erythromycins and azithromycins which have non-natural sugars at the C-5 position, in particular mycaminose and angolosamine. In a specific aspect the present invention provides for the synthesis of mycaminose and angolosamine using specific combinations of sugar biosynthetic genes in gene cassettes.

### SUMMARY OF THE INVENTION

The present invention relates to processes, and recombinant strains, for the preparation and isolation of erythromycins and azithromycins, which differ from the corresponding naturally occurring compound in the glycosylation of the C-5 position. In a specific aspect the present invention relates to processes, and recombinant strains, for the preparation and isolation of erythrcomycins and azithromycins, which incorporate angolosamine or mycaminose at the C-5 position. In particular, the present invention relates to processes and recombinant strains for the preparation and isolation of 5-O-dedesosaminyl-5-O- mycaminosyl, or angolosaminyl erythromycins and azithromycins, in particular 5-O-dedesosaminyl-5-O-mycaminosyl erythromycins and 5-O-dedesosaminyl-5-O-mycaminosyl azithromycins, and specifically 5-O-dedesosaminyl-5-O-mycaminosyl erythromycin B, 5-O-dedesosaminyl-5-O-mycaminosyl erythromycin C, 5-O-dedesosaminyl-5-O-mycaminosyl erythromycin D, 5-O-dedesosaminyl-5-O-mycaminosyl erythromycin A, and 5-O-dedesosaminyl-5-O-mycaminosyl azithromycin. The present invention further relates to novel 5-O-dedesosaminyl-5-O-mycaminosyl, angolosaminyl erythromycins and azithromycins produced thereby.

### DETAILED DESCRIPTION OF THE INVENTION

The present invention relates to processes, and recombinant strains, for the preparation and isolation of erythromycins and azithromycins which differ from the naturally occurring compound in the glycosylation of the C-5 position. These are referred to herein as “compounds of the invention” and unless the context dictates otherwise, such a reference includes a reference to 5-O-dedesosaminyl-5-O-mycaminosyl erythromycins, 5-O-dedesosaminyl-5-O-angolosaminyl erythromycins, 5-O-dedesosaminyl-5-O-mnycaminosyl azithromycins, and 5-O-dedesosaminyl-5-O-angolosaminyl azithromycins, specifically 5-O-dedesosaminyl-5-O-mycaminosyl erythromycin A, 5-O-dedesosaminyl-5-O-rnycaminosyl erythromycin C, 5-O-dedesosaminyl-5-O-mycaminosyl erythromycin B, 5-O-dedesosaminyl-5-O-mycaminosyl erythromycin D, 5-O-dedesosaminyl-5-O-mycaminosyl azithromycin, 5-O-dedesosaminyl-5-O-angolosaminyl erythromycin A, 5-O-dedesosaminyl-5-O-angolosaminyl erythromycin B, 5-O-dedesosaminyl-5-O- angolosaminyl erythromycin C, 5-O-dedesosaminyl-5-O-angolosaminyl erythromycin D, 5-O-dedesosaminyl-5-O-angolosaminyl azithromycin and analogues thereof which additionally vary in glycosylation at the C3 position (see WO 01/79520) and which may also vary in the aglycone backbones (see WO 98/01571, EP 1024145, WO 93/13663, WO 98/49315). The invention relates to processes, and recombinant strains, for the preparation and isolation of compounds of the invention. In particular, the present invention provides a process for the production of erythromycins and azithromycins which differ from the naturally occurring compound in the glycosylation of the C-5 position, said process comprising transforming a strain with a gene cassette as described herein and culturing the strain under appropriate conditions for the production of said erythromycin or azithrornycin. In a preferred embodiment the strain is an actinomycete, a pseudomonad, a myxobacterium, or an E. coli . In an alternative preferred embodiment the host strain is additionally transformed with the ermE gene from S. erythraea . In a more highly preferred embodiment, the host strain is an actinomycete. In a more highly preferred embodiment the host strain is selected from S. erytlraea, Streptomyces griseofuscus, Streptomyces cinnarnonensis, Streptomyces albus, Streptonmyces lividans, Streptomyces hygroscopicus sp., Streptomyces hygroscopicus var. ascomyceticus, Streptomyces longisporoflavus, Saccharopolyspora spinosa, Streptomyces tsukubaensis, Streptomyces coelicolor, Streptomyces fradiae, Streptomyces rimosus, Streptomyces avermitilis, Streptomyces eurythermzus, Streptomyces venezuelae, and Amycolatopsis mediterranei. In a specific embodiment the host strain is S. erythraea. In an alternative specific embodiment the host strain is selected from the SGQ2, Q42/1 or 18AI strains of S. erythraea.

The present invention further relates to novel 5-O-dedesosaminyl-5-O-angolosaminyl erythromycins and azithromycins produced thereby (FIG. 1). The methodology comprises in part the expression of a gene cassette in the S. erythraea mutant strain SGQ2 (which carries genomic deletions in eryA, eryCIII, eryBV and eryCIV (WO01/79520)), as described in Example 3 and 6 and in S. erythraea Q42/1 (BIOT-2166) (Examples 1-4) and S. erytliraea 18AI (BIOT-2634) (Example 6). Detailed descriptions are given in Examples 1-11.

The invention relates to a process involving the transformation of an actinomycete strain, including but not limited to strains of S. erythraea such as SGQ2, (see WO 01/79520) or Q42/1 or 18A1 (whose preparation is described below) with an expression plasmid containing a combination of genes which are able to direct the biosynthesis of a sugar moiety and direct its subsequent transfer to an aglycone or pseudoaglycone.

In a particular embodiment the present invention relates to a gene cassette containing a combination of genes which are able to direct the synthesis of mycaminose or angolosamine in an appropriate strain background.

In a particular embodiment the present invention relates to a gene cassette containing a combination of genes which are able to direct the synthesis of mycaminose in an appropriate strain background. The gene cassette may include genes selected from but not limited to angorf14, tylMIII, tylMI, tylB, tylAI, tylAII, tylia, angAI, angAII, angMIII, angB, angMI, eryG, eryK and glycosyltransferase genes including but not limited to tylMII, angMII, desVII, eryCIII, eryBV, spnP, and midI. In a preferred embodiment the gene cassette comprises tylia in combination with one or more other genes which are able to direct the synthesis of mycaminose. In a preferred embodiment the gene cassette comprises angorf14 in combination with one or more other genes which are able to direct the synthesis of mycaminose. In an more preferred embodiment the gene cassette comprises aTngAI, angAII, angorf14, angMIII, angB, angMI, in combination with one or more glycosyltransferases such as but not limited to eryCIII, tylMII, angMII, In an alternative embodiment the gene cassette comprises tylAI tylAII, tylMIII, tylB, tylIa, tylMI in combination with glycosyltransferases such as but not limited to eryCIII, tylMII and aiigMII. In a preferred embodiment the strain is an S. erythraea strain.

In a particular embodiment the present invention relates to a gene cassette containing combinations of genes which are able to direct the synthesis of angolosamine, including but not limited to angMIII, angMI, angB, anglAI angAII, angorf14, angorf4, tylMIII, tylMl, tylB, tyl4I tylAII, eryCVI, spnO, eryBVI, and eryK and one or more glycosyltransferase genes including but not limited to eryCIII, tylMII, angMII, des VII, eryBV, spnP and midi. In a preferred embodiment the gene cassette contains angMIII, angMI, angB, angAI angAII, angorf14, spnO in combination with a glycosyltransferase gene such as but not limited to angMII, tylMII or eryCIII. In an alternative preferred embodiment the gene cassette contains comprises angMIII, angMI, angB, angI; angAII, angorf4, and angorf14, in combination with one or more glycosyltransferases selected from the group consisting of angMII, tylMII and eryCIII. In a preferred embodiment the strain is an S. erythraea strain.

In one embodiment, the process of the present invention further involves feeding of an aglycone and/or a pseudoaglycone substrate (for definition see below), to the recombinant strain, said aglycone or pseudoaglycone is selected from the group including (but not limited to) 3-O-mycarosyl erythronolide B, erythronolide B, 6-deoxy erythronolide B, 3-O-mycarosyl-6-deoxy erythronolide B, tylactone, spinosyn pseudoaglycones, 3-O-rhamnosyl erythronolide B, 3-O-rhamnosyl-6-deoxy erythronolide B, 3-O-angolosaminyl erythronolide B, 15-hydroxy-3-O-mycarosyl erythronolide B, 15-hydroxy erythronolide B, 15-hydroxy-6-deoxy erythronolide B, 15-hydroxy-3-O-mycarosyl-6-deoxy erythronolide B, 15-hydroxy-3-O-rhamnosyl erythronolide B, 15-hydroxy-3-O-rhamnosyl-6-deoxy erythronolide B, 15-liydroxy-3-O-angolosaminyl erythronolide B, 14-hydroxy-3-O-mycarosyl erythronolide B, 14-hydroxy erythronolide B, 14-hydroxy-6-deoxy erythronolide B, 14-hydroxy-3-O-mycarosyl-6-deoxy erythronolide B, 14-hydroxy- 3-O-rhamnosyl erythronolide B, 14-hydroxy-3-O-rhamnosyl-6-deoxy erythronolide B, 14-hydroxy-3-O-angolosaminyl erythronolide B to cultures of the transformed actinomycete strains, the bioconversion of the substrate to compounds of the invention and optionally the isolation of said compounds. This process is exemplified in Examples 1-11. However, a person of skill in the art will appreciate that in an alternative embodiment the host cell can express the desired aglycone template, either naturally or recombinantly.

As used herein, the term “pseudoaglycone” refers to a partially glycosylated intermediate of a multiply-glycosylated product.

Those skilled in the art will appreciate that alternative host strains can be used. A preferred cell is a prokaryote or a fungal cell or a mammalian cell. A particularly preferred host cell is a prokaryote, more preferably host cell strains such as actinomycetes, Pseudomnonas, myxobacteria, and E. coli . It will be appreciated that if the host cell does not naturally produce erythromycin, or a closely related 14-membered macrolide, it may be necessary to introduce a gene conferring self-resistance to the macrolide product, such as the ermE gene from S. erythraea . Even more preferably the host cell is an actinomycete, even more preferably strains that include but are not limited to S. erythraea, Streptoiimyces griseofuscus, Streptomyces cinnamonensis, Streptomyces albus, Streptomyces lividans, Streptomyces hygroscopicus sp., Streptomyces hygroscopicus var. ascomyceticus, Streptomyces longisporoflavus, Saccharopolyspora spinosa, Streptomyces tsukubaensis, Streptomyces coelicolor, Streptomycesfradiae, Streptomyces rimosus, Streptomyces avermitilis, Streptomyces eurythermus, Streptomyces venezuelae, Amycolatopsis mediterranei. In a more highly preferred embodiment the host cell is S. erythraea.

It will readily occur to those skilled in the art that the substrate fed to the recombinant cultures of the invention need not be a natural intermediate in erythromycin biosynthesis. Thus, the substrate could be modified in the aglycone backbone (see Examples 8-11) or in the sugar attached at the 3-position or both. WO 01/79520 demonstrates that the desosaminyl transferase EryCIII exhibits relaxed specificity with respect to the pseudoaglycone substrate, converting 3-O-rhamnosyl erythronolides into the corresponding 3-O-rhamnosyl erythromycins. Appropriate modified substrates may also be produced by chemical semi-synthetic methods. Alternatively, methods of engineering the erythromycin-producing polyketide synthase, DEBS, to produce modified erythromycins are well known in the art (for example WO 93/13663, WO 98/01571, WO 98/01546, WO 98/49315, Kato, Y. et al., 2002). Likewise, WO 01/79520 describes methods for obtaining erythronolides with alternative sugars attached at the 3-position. Therefore, the term “compounds of the invention” includes all such non-natural aglycone compounds as described previous additionally with alternative sugars at the C-5 position. All these documents are incorporated herein by reference.

It will readily occur to those skilled in the art that the compounds of the invention containing a mycaminosyl moiety at the C-5 position could be modified at the C-4 hydroxyl group of the mycaminosyl moiety, including but not limited to glycosylation (see also WO 01/79520), acylation or chemical modification.

The present invention thus provides variants of erythromycin and related macrolides having at the 5-position a non-naturally occurring sugar, in particular an O-mycaminosyl, or O-angolosaminyl residue or a derivative or precursor thereof, specifically an O-angolosaminyl residue or a derivative thereof.

The term “variants of erythromycin” encompasses (a) erythromycins A, B, C and D; (b) semi-synthetic derivatives such as azithromycin and other derivatives as discussed in EP 1024145, which is incorporated herein by reference; (c) variants produced by genetic engineering and semi-synthetic derivatives thereof. Variants produced by genetic engineering include variants as taught in, or producible by, methods taught in WO 98/01571, EP 1024145, WO 93/13663, WO 98/49315 and WO 01/79520 which are incorporated herein by reference. The compounds of the invention include variants of erythromycin where the natural sugar at position C-5 has been replaced with mycaminose or angolosamine and also includes compounds of the following formulas (I—erythromycins and II—azithromycins) and pharmaceutically acceptable salts thereof. No stereochemistry is shown in Formula I or II as all possibilities are covered, including “natural” stereochemistries (as shown elsewhere in this specification) at some or all positions. In particular, the stereochemistry of any —CH(OH)— group is generally independently selectable.

### Formula I:

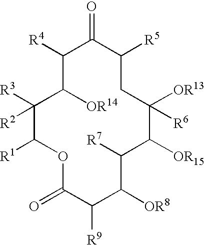

### Formula II

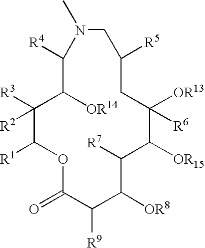 [0022] R¹═H, CH₃, C₂H₅ or is selected from i) below;
R², R⁴, R⁵, R⁶, R⁷ and R⁹ are each independently H, OH, CH₃, C₂H₅ or OCH₃;
R³═H or OH;
R⁸═H,

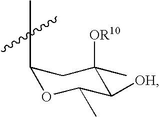

rhamnose, 2′-O-methyl rhamnose, 2′,3′-bis-O-methyl rhamnose, 2′,3′,4′-tri-O-methyl prhamnose, oleandrose, oliose, digitoxose, olivose or angolosamine;
R¹⁰═H, CH₃ or C(═O)RA, where R_{A}═C1-C6 alkyl, C2-C6 alkenyl or C2-C6 alkynyl;
R¹¹═H,

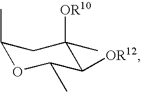

mycarose, C4-O-acyl-mycarose or glucose;
R¹²═H or C(═O)R_{A}, where R_{A}═C1-C6 alkyl, C2-C6 alkenyl or C2-C6 alkynyl;
R¹³═H or CH₃;
R¹⁵═H or

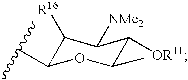

R¹⁶═H or OH;
R¹⁴═H or —C(O)NR^{c}R^{d}
   wherein each of R^{c} and R^{d} is independently H, C₁-C₁₀ alkyl, C₂-C₂₀ alkenyl, C₂-C₁₀ alkynyl, -(CH₂)ₘ(C₆-C₁₀ aryl), or —(CH₂)ₘ(5-10 membered heteroaryl), wherein m is an integer ranging from 0 to 4, and wherein each of the foregoing R^{c} and R^{d} groups, except H, may be substituted by 1 to 3 Q groups; or wherein R^{c} and R^{d} may be taken together to form a 4-7 membered saturated ring or a 5-10 membered heteroaryl ring, wherein said saturated and heteroaryl rings may include 1 or 2 heteroatoms selected from O, S and N, in addition to the nitrogen to which R^{c} and R^{d} are attached, and said saturated ring may include 1 or 2 carbon-carbon double or triple bonds, and said saturated and heteroaryl rings may be substituted by 1 to 3 Q groups; or R² and R¹⁷ taken together form a carbonate ring; each Q is independently selected from halo, cyano, nitro, trifluoromethyl, azido, —C(O)Q¹, —OC(O)Q¹, —C(O)OQ¹, —OC(O)OQ¹, —NQ²C(O)Q³, —C(O)NQ²Q³, —NQ²Q³, hydroxy, C₁-C₆ alkyl, C₁-C₆ alkoxy, —(CH₂)ₘ(C₆-C₁₀ aryl), and —(CH₂)ₘ(5-10 membered heteroaryl), wherein m is an integer ranging from 0 to 4, and wherein said aryl and heteroaryl substituents may be substituted by 1 or 2 substituents independently selected from halo, cyano, nitro, trifluoromethyl, azido, —C(O)Q¹, —C(O)OQ¹, —OC(O)OQ¹, —NQ²C(O)Q³, —C(O)NQ²Q³, —NQ²Q³, hydroxy, C₁-C₆ alkyl, and C₁-C₆ alkoxy;

each Q¹, Q² and Q³ is independently selected from H, OH, C₁-C₁₀ alkyl, C₁-C₆ alkoxy, C₂-C₁₀ alkenyl, C₂-C₁₀ alkynyl, —(CH₂)m(C₆-C₁₀ aryl), and —(CH₂)ₘ(5-10 membered heteroaryl), wherein m is an integer ranging from 0 to 4; with the proviso that the compound is not 5-O-dedesosaminyl-5-O- mycaminosyl erythromycin A or D.

The present invention also provides compounds according to formulas I or II above in which:

i) the substituent R¹ is selected from
an alpha-branched C₃-C₈ group selected from alkyl, alkenyl, alkynyl, alkoxyalkyl and alkylthioalkyl groups any of which may be optionally substituted by one or more hydroxyl groups;
a C₅-C₈ cycloalkylalkyl group wherein the alkyl group is an alpha-branched C₂-C₅ alkyl group;
a C₃-C₈ cycloalkyl group or C₅-C₈ cycloalkenyl group, either of which may optionally be substituted by one or more hydroxyl, or one or more C₁-C₄ alkyl groups or halo atoms;
a 3 to 6 membered oxygen or sulphur containing heterocyclic ring which may be saturated, or fully or partially unsaturated and which may optionally be substituted by one or more C₁-C₄ alkyl groups, halo atoms or hydroxyl groups;
phenyl which may be optionally substituted with at least one substituent selected from C₁-C₄ alkyl, C₁-C₄ alkoxy and C₁-C₄ alkylthio groups, halogen atoms, trifluoromethyl, and cyano or;
R¹ is R¹⁷—CH₂— where R¹⁷ is H, C₁-C₈ alkyl, C₂-C₈ alkenyl, C₂-C₈ alkynyl, alkoxyalkyl or alkylthioallkyl containing from 1 to 6 carbon atoms in each alkyl or alkoxy group wherein any of said alkyl, alkoxy, alkenyl or alkynyl groups may be substituted by one or more hydroxyl groups or by one or more halo atoms; or a C₃-C₈ cycloalkyl or C₅-C₈ cycloalkenyl either of which may be optionally substituted by one or more C₁-C₄ alkyl groups or halo atoms; or a 3 to 6 membered oxygen or sulphur containing heterocyclic ring which may be saturated or fully or partially unsaturated and which may optionally be substituted by one or more C₁-C₄ alkyl groups or halo atoms; or a group of the formula SA₁₆ wherein A₁₆ is C₁-C₈ alkyl, C₂-C₈ alkenyl, C₂-C₈ alkynyl, C₃-C₈ cycloalkyl, C₅-C₈ cycloalkenyl, phenyl or substituted phenyl wherein the substituent is C₁-C₄ alkyl, C₁-C₄ alkoxy or halo, or a 3 to 6 membered oxygen or sulphur-containing heterocyclic ring which may be saturated, or fully or partially unsaturated and which may optionally be substituted by one or more C₁-C₄ alkyl groups or halo atoms;

ii) the —CHOH— at CII (erythromycins) or C12 (azithromycins) is replaced by a methylene group (—CH₂—), a keto group (C═O), or by a 10,11-olefinic bond (erythromycins) or 11,12-olefinic bond (azithromycins);

iii) the substituent R¹¹ is H or mycarose or C4-O-acyl-mycarose or glucose; or compounds according to formula I or II above which differ in the oxidation state of one or more of the ketide units (i.e. selection of alternatives from the group: —CO—, —CH(OH)—, alkene —CH—, and CH₂) where the stereochemistry of any —CH(OH)— is also independently selectable, with the proviso that the compounds are not selected from the group consisting of 5-O-dedesosaminyl-5-O-mycaminosyl erythromycin A, 5-O-dedesosaminyl-5-O-mycaminosyl erythromycin D and 5-O-dedesosaminyl-5-O-mycaminnosyl azithromycin.

Novel 5-O-dedesosaminyl-5-O-angolosaminyl erythromycins and azithromycins made available by this aspect of the invention include, but are not limited to those where in the R¹⁵ group R¹¹═R¹⁶═H, with the proviso that they are not angolamycin or medermycin (Kinumaki and Suzuki, 1972; Ichinose et al., 2003).

In a preferred embodiment the present invention provides a compound according to formula I or II where: R¹═H, CH₃, C₂H₅ or selected from: an alpha-branched C₃-C₈ group selected from alkyl, alkenyl, alkynyl, alkoxyalkyl and alkylthioallcyl groups any of which may be optionally substituted by one or more hydroxyl groups; a C₅-C₈ cycloalkylalkyl group wherein the alkyl group is an alpha-branched C₂-C₅ alkyl group; a C₃-C₈ cycloalkyl group or C₅-C₈ cycloalkenyl group, either of which may optionally be substituted by one or more hydroxyl, or one or more C₁-C₄ alkyl groups or halo atoms; a 3 to 6 membered oxygen or sulphur containing heterocyclic ring which may be saturated, or fully or partially unsaturated and which may optionally be substituted by one or more C₁-C₄ alkyl groups, halo atoms or hydroxyl groups; phenyl which may be optionally substituted with at least one substituent selected from C₁-C₄ allcyl, C₁-C₄ alkoxy and C₁-C₄ alkylthio groups, halogen atoms, trifluoromethyl, and cyano or R¹ is R¹⁷-CH₂- where R¹⁷ is H, C₁-C₈ alkyl, C₂-C₈ alkenyl, C₂-C₈ alkynyl, alkoxyalkyl or alkylthioalkyl containing from 1 to 6 carbon atoms in each alkyl or alkoxy group wherein any of said alkyl, alkoxy, alkenyl or alkynyl groups may be substituted by one or more hydroxyl groups or by one or more halo atoms; or a C₃-C₈ cycloalkyl or C₅-C₈ cycloalkenyl either of which may be optionally substituted by one or more C- C₄ alkyl groups or halo atoms; or a 3 to 6 membered oxygen or sulphur containing heterocyclic ring which may be saturated or fully or partially unsaturated and which may optionally be substituted by one or more C₁-C₄ alkyl groups or halo atoms; or a group of the formula SA₁₆ wherein A₁₆ is Cl-C₈ alkyl, C₂- C₈ alkenyl, C₂-C₈ alkynyl, C₃-C₈ cycloalkyl, C5-C8 cycloalkenyl, phenyl or substituted phenyl wherein the substituent is C₁ -C₄ alkyl, C₁-C₄ alkoxy or halo, or a 3 to 6 membered oxygen or sulphur-containing heterocyclic ring which may be saturated, or fully or partially unsaturated and which may optionally be substituted by one or more C₁-C₄ allcyl groups or halo atoms
R², R⁴, R⁵, R⁶, R⁷ and R⁹ are all CH₃
R³ is H or OH
R⁸═H or

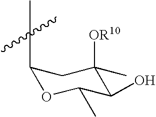

or is selected from rhamnose, 2′-O-methyl rhamnose, 2′,3′-bis-O-methyl rhamnose, 2′,3′,4′-tri-O-methyl rhamnose, oleandrose, oliose, digitoxose, olivose and angolosamine;
R¹⁰═H or CH3
R¹¹═H or

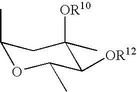

R¹²═H or C(═O)R_{A}, where R_{A}═C1-C6 alkyl, C2-C6 alkenyl or C2-C6 alkynyl
R¹³═H or CH₃
R¹⁴═H or —C(O)NR^{c}R^{d} wherein each of R^{c} and R^{d} is independently H, C₁-C₁₀ alkyl, C₂-C₂₀ alkenyl, C₂-C₁₀ alkynyl, —(CH₂)ₘ(C₆-C₁₀ aryl), or —(CH₂)ₘ(5-10 membered heteroaryl), wherein m is an integer ranging from 0 to 4, and wherein each of the foregoing R^{c} and R^{d} groups, except H, may be substituted by

1 to 3 Q groups; or wherein R^{c} and R^{d} may be taken together to form a 4-7 membered saturated ring or a 5-10 membered heteroaryl ring, wherein said saturated and heteroaryl rings may include 1 or 2 heteroatoms selected from 0, S and N, in addition to the nitrogen to which RC and Rd are attached, and said saturated ring may include 1 or 2 carbon-carbon double or triple bonds, and said saturated and heteroaryl rings may be substituted by 1 to 3 Q groups; or R² and R¹⁷ taken together form a carbonate ring; each Q is independently selected from halo, cyano, nitro, trifluorornethyl, azido, —C(O)Q¹, —OC(O)Q¹, —C(O)OQ¹, —OC(O)OQ¹, —NQ²C(O)Q³, —C(O)NQ²Q³, —NQ²Q³, hydroxy, C₁-C₆ alkyl, C₁-C₆ alkoxy, —(CH₂)ₘ(C₆-C₁₀ aryl), and —(CH₂)ₘ(5-10 membered heteroaryl), wherein m is an integer ranging from 0 to 4, and wherein said aryl and heteroaryl substituents may be substituted by 1 or 2 substituents independently selected from halo, cyano, nitro, trifluoromethyl, azido, —C(O)Q¹, —C(O)OQ¹, —OC(O)OQ¹, —NQ²C(O)Q³, —C(O)NQ²Q³, —NQ²Q³, hydroxy, C₁-C₆ alkyl, and C₁-C₆ alkoxy;

each Q¹, Q² and Q³ is independently selected from H, OH, C₁-C₁₀ alkyl, C₁-C₆ alkoxy, C₂-C₁₀ alklenyl, C₂-C₁₀ alkynyl, —(CH₂)ₘ(C₆-C₁₀ aryl), and —CH₂)ₘ(5-10 membered heteroaryl), wherein m is an integer ranging from 0 to 4; with the proviso that the compound is not 5-O-dedesosaminyl-5-O-mycaminosyl erythromycin A or D

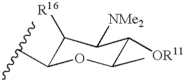

R¹⁵═H or
R¹⁶═H or OH
   with the proviso that the compounds are not selected from the group consisting of 5-O-dedesosaminyl-5-O-mycaminosyl erythromycin A, 5-O-dedesosaminyl-5-O-mycaminosyl erythromycin D and 5-O-dedesosaminyl-5-O-mycaminosyl azithromycin

In a further preferred embodiment the present invention provides a compound according to formula 1, wherein:
R¹═H, CH₃, C₂H₅ or selected from: an alpha-branched C₃-C₈ group selected from alkyl, alkenyl, alkynyl, alkoxyallcyl and alkylthioalkyl groups any of which may be optionally substituted by one or more hydroxyl groups; a C₅-C₈ cycloalkylalkyl group wherein the alkyl group is an alpha-branched C₂-C₅ alkyl group; a C₃-C₈ cycloalkyl group or C₅-C₈ cycloalkenyl group, either of which may optionally be substituted by one or more hydroxyl, or one or more C₁-C₄ alkyl groups or halo atoms; a 3 to 6 membered oxygen or sulphur containing heterocyclic ring which may be saturated, or fully or partially unsaturated and which may optionally be substituted by one or more C₁-C₄ alkyl groups, halo atoms or hydroxyl groups; phenyl which may be optionally substituted with at least one substituent selected from C₁-C₄ alkyl, C₁-C₄ alkoxy and C₁-C₄ alkylthio groups, halogen atoms, trifluoromethyl, and cyano or R¹ is R¹⁷-CH₂- where R¹⁷ is H, C₁-C₈ alkyl, C₂-C₈ alkenyl, C₂-C₈ alkynyl, alkoxyalkyl or alkylthioalkyl containing from 1 to 6 carbon atoms in each alkyl or alkoxy group wherein any of said alkyl, alkoxy, alkenyl or allkynyl groups may be substituted by one or more hydroxyl groups or by one or more halo atoms; or a C₃-C₈ cycloallcyl or C₅-C₈ cycloalkenyl either of which may be optionally substituted by one or more C₁-C₄ alkyl groups or halo atoms; or a 3 to 6 membered oxygen or sulphur containing heterocyclic ring which may be saturated or fully or partially unsaturated and which may optionally be substituted by one or more C₁-C₄ alkyl groups or halo atoms; or a group of the formula SA₁₆ wherein A₁₆ is C₁-C₈ all,yl, C₂-C₈ allkenyl, C₂-C₈ alkynyl, C₃-C₈ cycloalkyl, C₅-C₈ cycloalkenyl, phenyl or substituted phenyl wherein the substituent is C₁-C₄ alkyl, C₁-C₄ alkoxy or halo, or a 3 to 6 membered oxygen or sulphur-containing heterocyclic ring which may be saturated, or fully or partially unsaturated and which may optionally be substituted by one or more C₁-C₄ alkyl groups or halo atoms
R²) R⁴, R⁵, R⁶, R⁷ and R⁹ are all CH₃
R³is H or OH
R⁸═H or

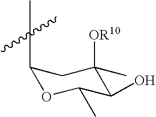

or is selected from rhamnose, 2′-O-methyl rhamnose, 2′,3′-bis-O-methyl rhamnose, 2′,3′,4′-tri-O-methyl rhamnose, oleandrose, oliose, digitoxose, olivose and angolosamine;
R10═H or CH3
R¹¹═H or

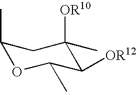

R¹²═H or C(═O)R_{A}, where R_{A}═C1-C6 alkyl, C2-C6 alkenyl or C2-C6 alkynyl
R¹³═H or CH₃
R¹⁴═H
R¹⁵═H or

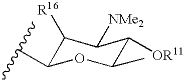

R¹⁶═H or OH
   with the proviso that the compounds are not selected from the group consisting of 5-O-dedesosaminyl-5-O-mycaminosyl erythromycin A, 5-O-dedesosaminyl-5-O-mycaminosyl erythromycin D and 5-O-dedesosam inyl-5-O-mycaminosyl azithromycin

In a more preferred embodiment the present invention provides a compound according to formula I where:
R¹═C₂H₅ optionally substituted with a hydroxyl group
R², R⁴ , R ⁶, R⁷ and R⁹ are all CH₃
R³ is H or OH
R⁸═H or

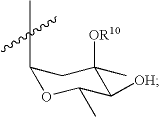

R¹⁰═H or CH₃
R¹¹═H or

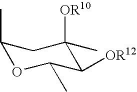

R¹²═H or C(═O)R_{A}, where R_{A}═C1-C6 alkyl, C2-C6 alkenyl or C2-C6 alkynyl
R¹³═H or CH₃
R¹⁴═H
R¹⁵═H or

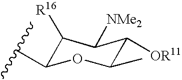

R¹⁶═H or OH
   with the provisio that the compounds are not selected from the group consisting of 5-O-dedesosaminyl-1-5-O-mycaminosyl erythromycin A and 5-O-dedesosaminyl-5-O-mycaminosyl erythromycin D

In a more preferred embodiment the present invention provides a compound according to formula I where
R¹═C₂h₅ optionally substituted with a hydroxyl group
R², R⁴, R⁵, R⁶, R⁷ and R⁹ are all CH₃
R³ is H or OH
R⁸═H or

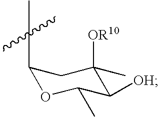

R¹⁰═H or CH₃
R¹²═H
R¹³═H or CH₃
R¹⁴═H
R¹⁵═H or

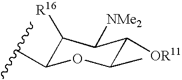

R¹⁶═H or OH

In a highly preferred embodiment the present invention provides a compound according to formula I where
R¹═C₂H₅
R², R⁴, R⁵, R⁶, R⁷ and R⁹ are all CH₃
R³ is H or OH
R⁸═H or

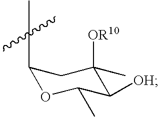

R¹⁰═H or CH₃
R¹²═H
R¹³═H or CH₃
R¹⁴═H
R¹⁵ ═H or

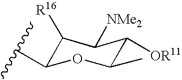

R¹⁶═H or OH
   with the proviso that the compounds are not selected from the group consisting of 5-O-dedesosaminyl-5-O-mycaminosyl erythromycin A and 5-O-dedesosaminyl-5-O-mycaminosyl erythromycin D.

Additionally, a person of skill in the art will appreciate that, using the methods of the present invention, mycaminose and angolosamine may be added to other aglycones or pseudoaglycones for example (but without limitation) a tylactone or spinosyn pseudoaglycone. These other aglycones or pseudoaglycones may be the naturally occurring structure or they may be modified in the aglycone backbone, such modified substrates may be produced by chemical semi-synthetic methods (Kaneko et al., 2000 and references cited therein). or, alternatively, via PKS engineering, such methods are well known in the art (for example WO 93/13663, WO 98/01571, WO 98/01546, WO 98/49315, Kato, Y. et al., 2002). Therefore, in a further embodiment the present invention provides 5-O-angolosaminyl tylactone, 5-O-mycaminosyl tylactone, 17-O-angolosaminyl spinosyn and 17-O-mycaminosyl spinosyn.

Moreover, the process of the host cell selection further comprises the optional step of deleting or inactivating or adding or manipulating genes in the host cell. This process comprises the improvement of recombinant host strains for the preparation and isolation of compounds of the invention, in particular 5-O-dedesosamiinyl-5-O-mycaminosyl erythromycins and 5-O-dedesosaminyl-5-O-mycaminosyl azithromycins, specifically 5-O-dedesosaminyl-5-O-mycaminosyl erythromycin A, 5-O-dedesosaminyl- 5-O-inycaminosyl erythromycin C, 5-O-dedesosaminyl-5-O-mycaminosyl erythromycin B, 5-O- dedesosamninyl-5-O-mycaminosyl erythromycin D and 5-O-dedesosaminyl-5-O-mycaminosyl azithromycin. This approach is exemplified in Example 1 by introducing an eryBVI mutation into the chromosome of S. erythraea SGQ2 in order to optimise the conversion of the substrate 3-O-mycarosyl erythlonlolide B to 5-O-dedesosaminyl-5-O-mycaminosyl erythromycins.

In a further aspect the invention relates to the construction of gene cassettes. The cloning method used to isolate these gene cassettes is analogous to that used in PCT/GBO3/003230 and diverges significantly from the approach previously described (WO 01/79520) by assembling the gene cassette directly in an expression vector rather than pre-assembling the genes in pUC18/19 plasmids, thus providing a more rapid cloning procedure for the isolation of gene cassettes. The strategy for isolating these gene cassettes is exemplified in Example 1 to Example 11. A schematic overview of the strategy is given in FIG. 2.

Another aspect of the invention allows the enhancement of gene expression by changing the order of genes in a gene cassette, the genes including but not limited to tylMI, tylMIII, tylB, eryCVI, tylAI, tylAII, eryCIII, eryBV, augAl, angAII, angMIII, angB, angMI, angorf14, angorf4, eryBVI, eryK, eryG, angMII, tylMII, desVII,,midI, spnO, spnN, spnP and genes with similar functions, allowing the arrangement of the genes in a multitude of permutations (FIG. 2).

The cloning strategy outlined in this invention also allows the introduction of a histidine tag in combination with a terminator sequence 3′ of the gene cassette to enhance gene expression (see Example 1). Those skilled in the art will appreciate other terminator sequences well known in the art could be used. See, for example Bussiere and Bastia (1999), Bertram et al, (2001) and Kieser et al. (2000), incorporated herein by reference.

Another aspect of the invention comprises the use of alternative promoters such as PtipA (Ali et al., 2002) and/or Pptr (Salah-Bey et al., 1995) to express genes and/or assembled gene cassette(s) to enhance expression.

Another aspect of the invention describes the multiple uses of promoter sequences in the assembled gene cassette to enhance gene expression as exemplified in Example 6.

Another aspect of the invention describes the addition of genes encoding for a NDP-glucose-synthase such as tylAI and a NDP-glucose-4,6-dehydratase such as tylAIJ to the gene cassette in order to enhance the endogenous production of the activated sugar substrate. Those skilled in the art will appreciate that alternative sources of equivalent sugar biosynthetic pathway genes may be used. In this context alternative sources include but are not limited to:

TylAI- homologues: DesIII of Streptomyces venezuelae (accession no AAC68682), GrsD of Streptomyces griseus (accession no AAD31799), AveBIII of Streptomyces avermitilis (accession no BAA84594), Gtt of Saccharopolyspora spinosa (accession no AAK83289), SnogJ of Streptomyces nogalater (accession no AAF01820), AclY of Streptoniyces galilaeus (accession no BAB72036), LanG of Streptonmyces cyanogenus (accession no AAD13545), Graorf16(GraD) of Streptomyces violaceoruber (accession no AAA99940), OleS of Streptomyces antibioticus (accession no AAD55453) and StrD of Streptoniyces griseus (accession no A26984) and AngAI of S. eurythermus.

TylAII- homologues: AprE of Streptomyces tenebrarius (accession no AAG18457), GdH of S. spinosa (accession no AAK83290), DesIV of S. venezuelae (accession no AAC68681), GdH of S. erytlhraea (accession no AAA68211), AveBII of S. avermitilis (accession no BAA84593), Scf81.08C of Streptomyces coelicolor (accession no CAB61555), LanH of S. cyanogenus (accession no AAD13546), Graorf17 (GraE) of S. violaceoruber (accession no S58686), OleE of S. antibioticus (accession no AAD55454), StrE of S. griseus (accession no P29782) and AngAIl of S. eurythermnus.

Similarly, alternative sources for activated sugar biosynthesis gene homologues to tylMIII, angAIII, eryCII, tjMII, angMII, tylB, angB, eryCI, tylMI, aingMI, eryCVI, tylIa, angorf14, angorf4, spnO, eryBVI, eryBV, eryCIII, desVII, midI, spnN and spnP will readily occur to those skilled in the art, and can be used.

Another aspect of the invention describes the use of alternative glycosyltransferases in the gene cassettes such as EryCIII. Those skilled in the art will appreciate that alternative glycosyltransferases may be used. In this context alternative glycosyltransferases include but are not limited to: TylMII (Accession no CAA57472), DesVII (Accession noAAC68677), MegCIII (Accession no AAG13921), MegDI (Accession no AAG13908) or AngMII of S. eurythermus.

In one aspect of the present invention, the gene cassette may additionally comprise a chimeric glycosyltransferase (GT). This is particularly of benefit where the natural GT does not recognise the combination of sugar and aglycone that is required for the synthesis of the desired analogue. Therefore, in this aspect the present invention specifically contemplates the use of a chimearic GT wherein part of the GT is specific for the recognition of the sugar whose synthesis is directed by the genes in said expression cassette when expressed in an appropriate strain background and part of the GT is specific for the aglycone or pseudoaglycone template (Hu and Walker, 2002).

Those skilled in the art will appreciate that different strategies may be used for the introduction of gene cassettes into the host strain, such as site-specific integration vectors (Smovkina et al., 1990; Lee et al., 1991; Matsuura et al., 1996; Van Mel laert et al., 1998; Kieser et al., 2000). Alternatively, plasmids containing the gene cassettes may be integrated into any neutral site on the chromosome using homologous recombination sites. Further, for a number of actinomycete host strains, including S. erythraea, the gene cassettes may be introduced on self-replicating plasmids (Kieser et al., 2000; WO 98/01571).

A further aspect of the invention provides a process for the production of compounds of the invention and optionally for the isolation of said compounds.

A further aspect of the invention is the use of different fermentation methods to optimise the production of the compounds of the invention as exemplified in Example 1. Another aspect of the invention is the addition of ery genes such as eryK and/or eryG into the gene cassette. One skilled in the art will appreciate that the process can be optimised for the production of a specific erythromycin (i.e. A, B, C, D) or azithromycin by manipulation of the genes eryG (responsible for the methylation on the mycarose sugar) and/or eryK (responsible for hydroxylation at C12). Thus, to optimise the production of the A-form, an extra copy of eryK may be included into the gene cassette. Conversely, if the erythromycin B analogue is required, this can be achieved by deletion of the eryK gene from the S. erythraea host strain, or by working in a heterologous host in which the gene and/or its functional homologue, is not present. Similarly, if the erythromycin D analogue is required, this can be achieved by deletion of both eryG and eryK genes from the S. erythraea host strain, or by working in a heterologous host in which both genes and/or their functional homologues are not present. Similarly, if the erythromycin C analogue is required, this can be achieved by deletion of the eryG gene from the S. erythraea host strain, or by working in a heterologous host in which the gene and/or its functional homologues are not present.

In this context a preferred host cell strain is a mammalian cell strain, fungal cells strain or a prokaryote. More preferably the host cell strain is an actinomycete, a Pseudomonad, a myxobacterium or an E. coli . In a more preferred embodiment the host cell strain is an actinomycete, still more preferably including, but not limited to Saccharopolyspora erythraea, Streptomyces coelicolor, Streptomyces avermitilis, Streptomyces griseofuscus, Streptomyces cinnarnonensis, Streptomycesfradiae, Streptomyces eurythermus, Streptomyces longisporofiavus, Streptomyces hygroscopicus, Saccharopolyspora spinosa, Micromnonospora griseorubida, Streptomyces lasaliensis, Streptomyces venezuelae, Streptomyces antibioticzus, Streptomyces lividans, Streptomyces rimosus, Streptomyces albus, Amycolatopsis mediterranei, Nocardia sp, Streptomyces tsukubaensis and Actinoplanes sp. N902-109. In a still more preferred embodiment the host cell strain is selected from Saccharopolyspora erythraea, Streptomyces griseofuescus, Streptomyces cinnamonensis, Streptomyces albus, Streptomyces lividans, Streptomyces hygroscopicus sp., Streptomyces hygroscopicus var. ascoinyceticus, Streptomyces longisporoflavus, Saccharopolyspora spinosa, Streptomyces tsukubaensis, Streptomyces coelicolor, Streptomyces fradiae, Streptomyces rimosus, Streptomyces avermitilis, Streptomyces eurythermus, Streptomyces venezuelae, Amycolatopsis mediterranei. In the most highly preferred embodiment the host strain is Saccharopolyspora erythraea.

The present invention provides methods for the production and isolation of compounds of the invention, in particular of erythromycin and azithromycin analogues which differ from the natural compound in the glycosylation of the C-5 position, for example but without limitation: novel 5-O-dedesosaminyl-5-O-mycaminosyl or angolosaminyl erythromycins and 5-O-dedesosaminyl-5-O-mycaminosyl, or angolosaminyl azithromycins which are useful as anti-microbial agents for use in human or animal health.

In further aspects the present invention provides novel products as obtainable by any of the processes disclosed herein.

### BRIEF DESCRIPTION OF FIGURES

FIG. 1A. Structures of 5-O-dedesosaminyl-5-O-mycaminosyl erythromycin A, 5-O-dedesosaminyl-5-O-mycaminosyl erythromycin B and 5-O-dedesosaminyl-5-O-mycaminosyl erythromycin C.

FIG. 1B. Structure of 5-O-dedesosaminyl-5-O-mycaminosyl azithromycin.

FIG. 2: Schematic overview over the gene cassette cloning strategy. Vector pSG144 was derived from vector pSG142 (Gaisser et al., 2000). Abbreviations: dam⁻: DNA isolated from dam⁻ strain background, XbaI^{met}:XbaI site sensitive to Dam methylation, eryR-HS:DNA fragment of the right hand side of the ery-cluster as described previously (Gaisser et al., 2000).

FIG. 3: Amino acid comparison between the published sequence of TylA1 (below, SEQ ID NO: 1) and the amino acid sequence detected from the sequencing data described in this invention (above, SEQ ID NO: 2). The changes in the amino acid sequence are underlined.

FIG. 4: Amino acid comparison between the published sequence of TylAII (below, SEQ ID NO: 3) and the amino acid sequence detected from the sequencing data described in this invention (above, SEQ ID NO: 4). The changes in the amino acid sequence are underlined.

FIG. 5: Structure of 5-O-angolosaminyl tylactone.

FIG. 6: Shows an overview of the angolamycin polyketide synthase gene cluster.

FIG. 7: The DNA sequence which comprises orf14 and orf15 (angB) from the angolamycin gene cluster (SEQ ID NO: 5).

FIG. 8: The DNA sequence which comprises orf2 (angAI), orf3 (angAII) and orf4 from the angolamycin gene cluster (SEQ ID NO: 6).

FIG. 9: The DNA sequence which comprises orf1* (angMIII), orj2* (angMII), and orf3* (angMI) from the angolamycin gene cluster (SEQ ID NO: 7).

FIG. 10: The amino acid sequence which corresponds to orf2 (angAI, SEQ ID NO: 8).

FIG. 11: The amino acid sequence which corresponds to orf3 (angAII, SEQ ID NO: 9).

FIG. 12: The amino acid sequence which corresponds to orf4 (SEQ ID NO: 10)

FIG. 13: The amino acid sequence which corresponds to orf14 (SEQ ID NO: 11).

FIG. 14: The amino acid sequence which corresponds to orf15 (angB, SEQ ID NO: 12).

FIG. 15: The amino acid sequence which corresponds to orf1* (angMIII, SEQ ID NO: 13).

FIG. 16: The amino acid sequence which corresponds to orf2* (angMII, SEQ ID NO: 14).

FIG. 17: The amino acid sequence which corresponds to oif3* (angMI, SEQ ID NO: 15).

### GENERAL METHODS

Escherichia coli XL1-Blue MR (Stratagene), E. coli DH10B (GibcoBRL) and E. coli ET12567 were grown in 2xTY medium as described by Sambrook et al., (1989). Vector pUC18, pUC19 and Litmus 28 were obtained from New England Biolabs. E. coli transformants were selected with 100 μg/mL ainpicillin. Conditions used for growing the Saccharopolyspora erythraea NRRL 2338-red variant strain were as described previously (Gaisser et al., 1997, Gaisser et al., 1998). Expression vectors in S. erythraea were derived from plasmid pSG142 (Gaisser et al., 2000). Plasmid-containing S. erythraea were selected with 25-40 μg/mL thiostrepton or 50 μg/mL apramycin. To investigate the production of antibiotics, S. erythraea strains were grown in sucrose-succinate medium (Caffrey et al., 1992) as described previously (Gaisser et al., 1997) and the cells were harvested by centrifugation. Chromosomal DNA of Streptomyces rochei ATCC21250 was isolated using standard procedures (Kieser et al., 2000). Feedings of 3-O-mycarosyl erythronolide B or tylactone were carried out at concentrations between 25 to 50 mg /L.

### DNA Manipulation and Sequencing

DNA manipulations, PCR and electroporation procedures were carried out as described in Sambrook et al., (1989). Protoplast formation and transformation procedures of S. erythraea were as described previously (Gaisser et al., 1997). Southern hybridizations were carried out with probes labelled with digoxigenin using the DIG DNA labelling kit (Boehringer Mannheim). DNA sequencing was performed as described previously (Gaisser et al., 1997), using automated DNA sequencing on double stranded DNA templates with an ABI Prism 3700 DNA Analyzer. Sequence data were analysed using standard programs.

### Extraction and Mass Spectrometry

1 mL of each fermentation broth was harvested and the pH was adjusted to pH 9. For extractions an equal volume of ethyl acetate, methanol or acetonitrile was added, mixed for at least 30 min and centrifuged. For extractions with ethyl acetate, the organic layer was evaporated to dryness and then re-dissolved in 0.5 mL methanol. For methanol and acetonitrile extractions, supernatant was collected after centrifugation and used for analysis. High resolution spectra were obtained on a Bruker BioApex II FT-ICR (Bruker, Bremen, FRG).

### Analysis of Culture Broths

An aliquot of whole broth (1 mL) was shaken with CH₃CN (1 mL) for 30 minutes. The mixture was clarified by centrifugation and the supernatant analysed by LCMS. The HPLC system comprised an Agilent HP1100 equipped with a Luna 5 μm C18 BDS 4.6×250 mm column (Phenomenex, Macclesfield, UK) heated to 40° C. The gradient elution was from 25% mobile phase B to 75% mobile phase B over 19 minutes at a flow rate of 1 mL/min. Mobile phase A was 10% acetonitrile: 90% water, containing 10 mM ammonium acetate and 0.15% formic acid, mobile phase B was 90% acetonitrile: 10% water, containing 10 mM ammonium acetate and 0.15% formic acid. The HPLC system described was coupled to a Bruker Daltonics Esquire3000 electrospray mass spectrometer operating in positive ion mode.

### Extraction and Purification Protocol:

For NMR analysis of 5-O-dedesosaminyl-5-O-mycaminosyl erythromycin A the fermentation broth was clarified by centrifugation to provide supernatant and cells. The supernatant was applied to a column (16×15 cm) of Diaione HP20 resin (Supelco), washed with 10% Me₂CO/H₂0 (2×2 L) and then eluted with Me₂CO (3.5 L). The cells were mixed to homogeneity with an equal volume of Me₂CO/MeOH (1:1). After at least 30 minutes the slurry was clarified by centrifugation and the supernatant decanted. The pelleted cells were similarly extracted once more with Me₂CO/MeOH (1:1). The cell extracts were combined with the Me₂CO from the HP20 column and the solvent was removed in vacuo to give an aqueous concentrate. The aqueous was extracted with EtOAc (3×) and the solvent removed in vacuo to give a crude extract. The residue was dissolved in CH₃CN/MeOH and purified by repeated rounds of reverse phase (C18) high performance liquid chromatography using a Gilson HPLC, eluting a Phenomenex 21.2×250 mm Luna 5 μm C18 BDS column at 21 mL/min. Elution with a linear gradient of 32.5% B to 63% B was used to concentrate the macrolides followed by isocratic elution with 30% B to resolve the individual erythromycins. Mobile phase A was 20 mM ammonium acetate and mobile phase B was acetonitrile. High resolution mass spectra were acquired on a Bruker BioApex II FTICR (Bruker, Bremen, Germany).

For NMR analysis of 5-O-angolosaminyl tylactone bioconversion experiments were performed as previously described with four 2 L flasks containing each 400 mL of SSDM medium inoculated with 5% of pre-cultures. Feedings with tylactone were carried out at 50 mg/L. The culture was centrifuged and the pH of the supernatant was adjusted to about pH 9 followed by extractions with three equal volumes of ethyl acetate. The cell pellet was extracted twice with equal volumes of a mixture of acetone-methanol (50:50, vol/vol). The extracts were combined and concentrated in vacuo. The resulting aqueous fraction was extracted three times with ethyl acetate and the extracts were combined and evaporated until dryness.

This semi purified extract was dissolved in methanol and purified by preparative HPLC on a Gilson 315 system using a 21 mm×250 mm Prodigy ODS3 column (Phenomenex, Macclesfield, UK). The mobile phase was pumped at a flow rate of 21 mL/min as a binary system consisting of 30% CH₃CN, 70% H₂0 increasing linearly to 70% CH₃CN over 20 min.

### Sequence Information

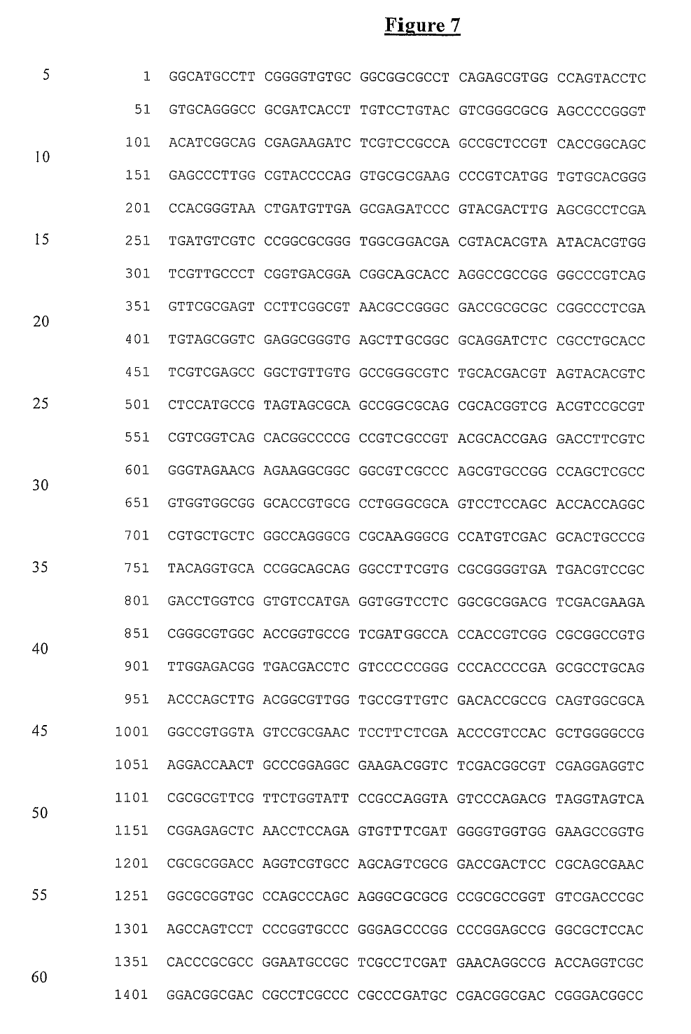

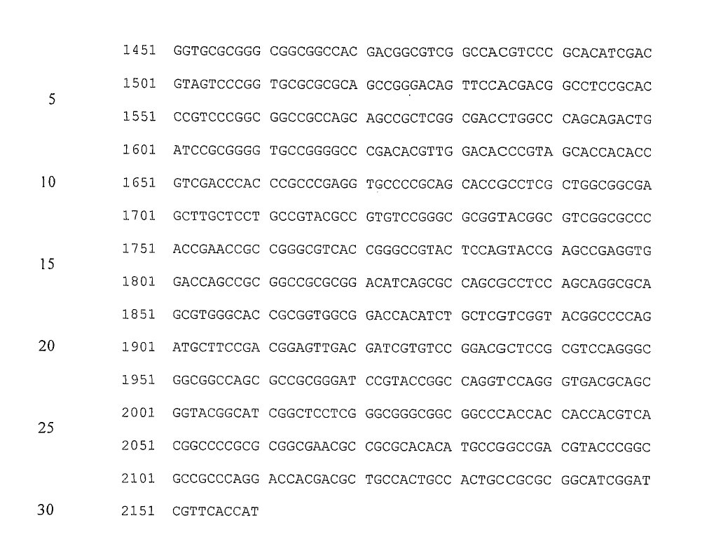

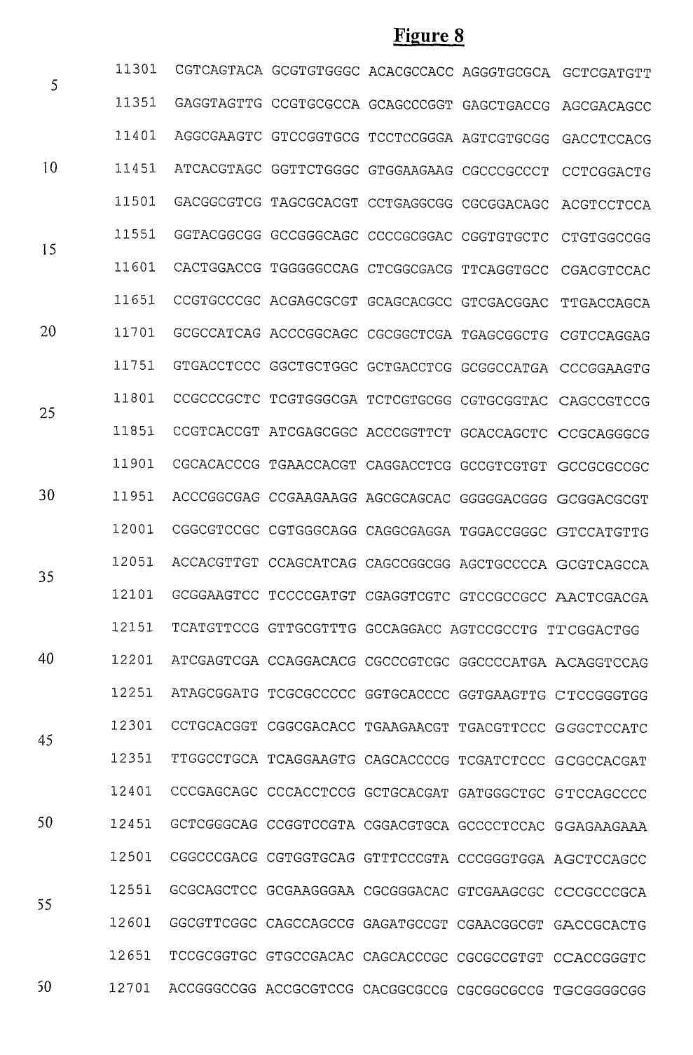

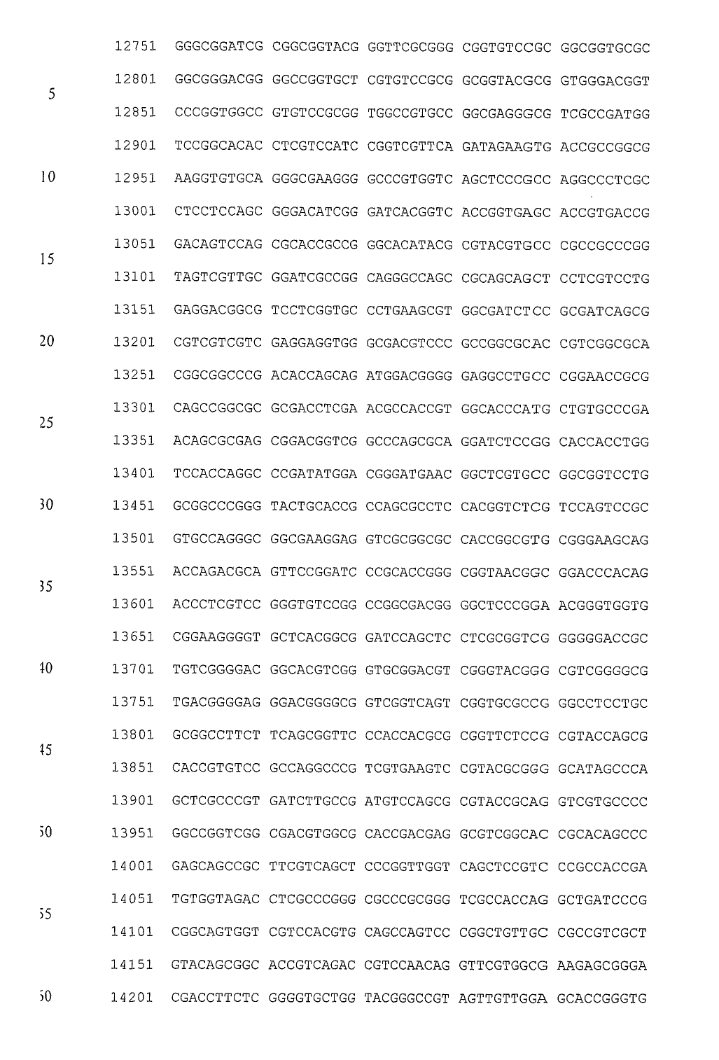

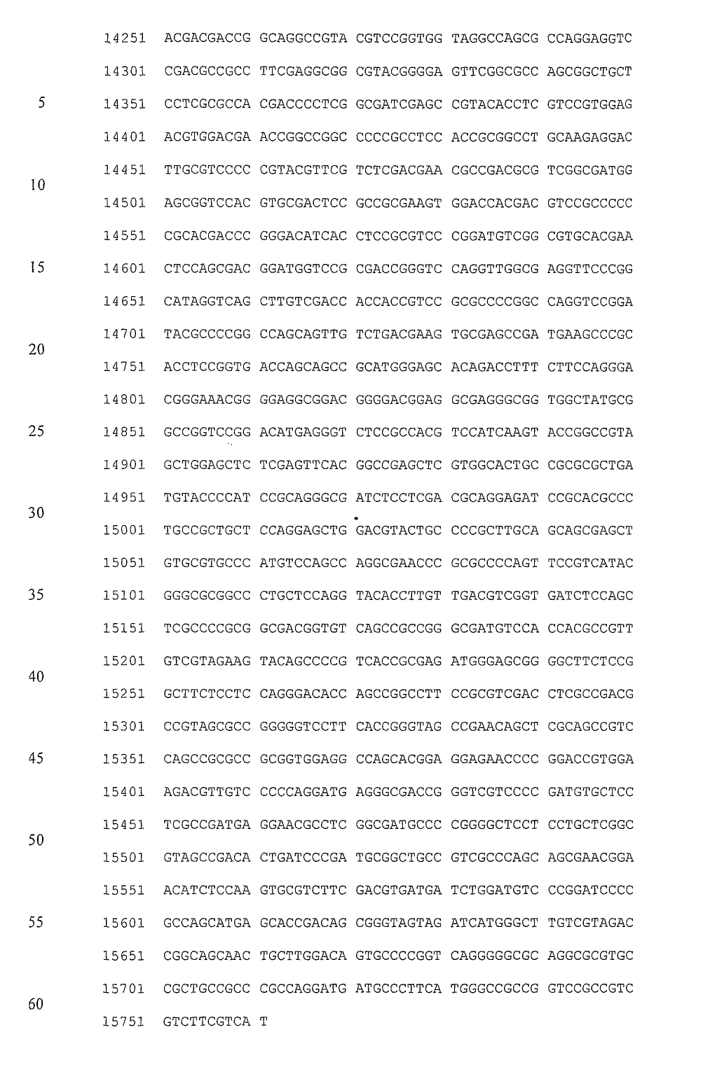

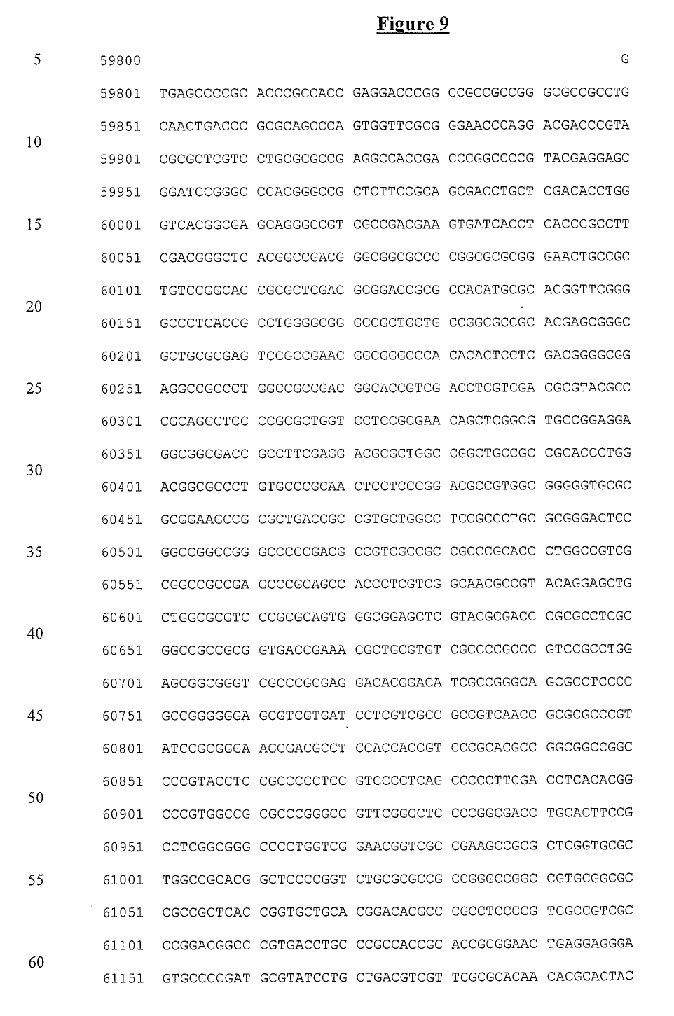

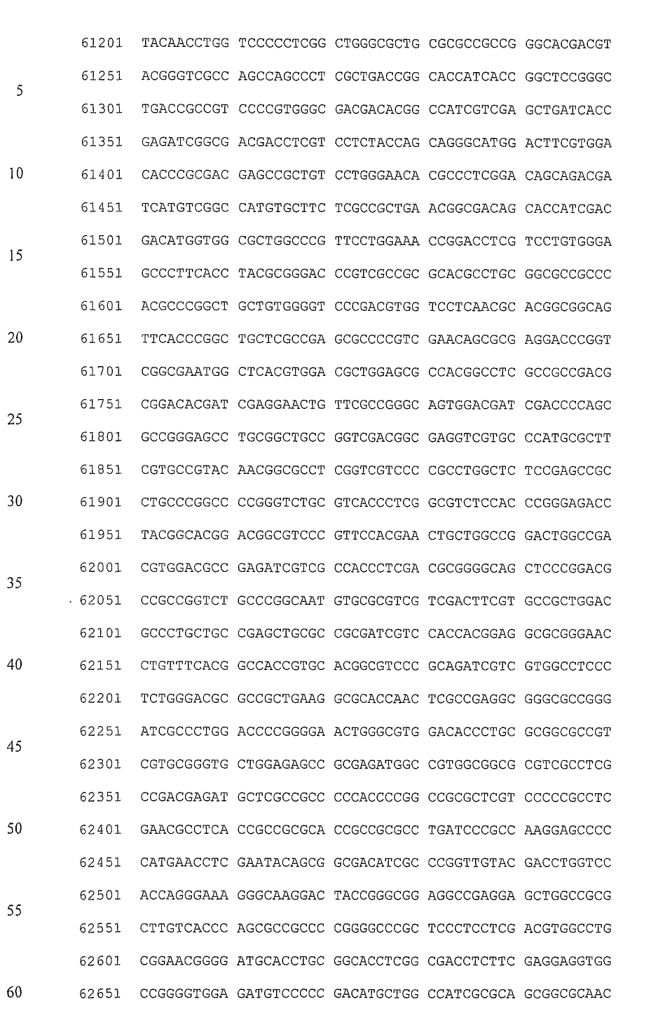

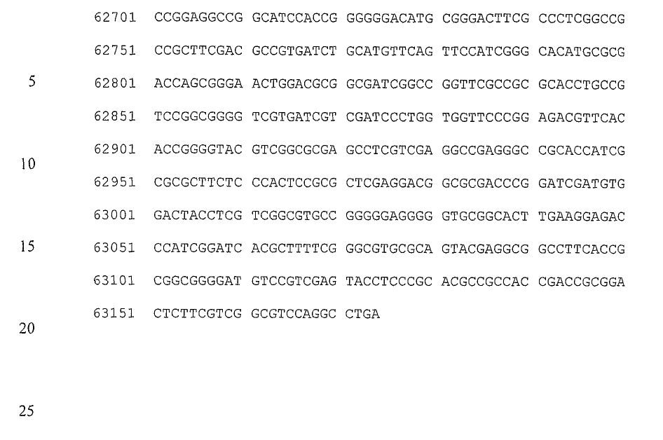

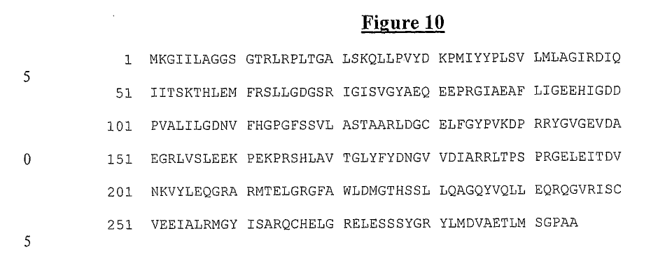

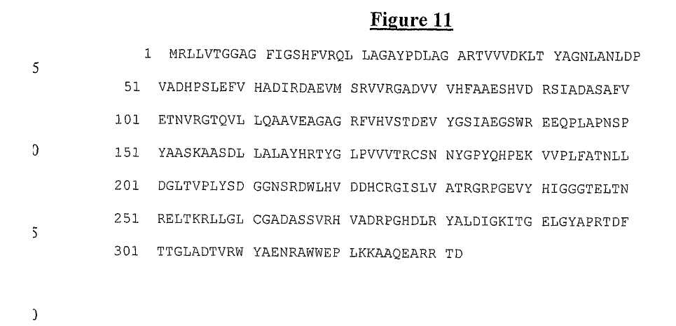

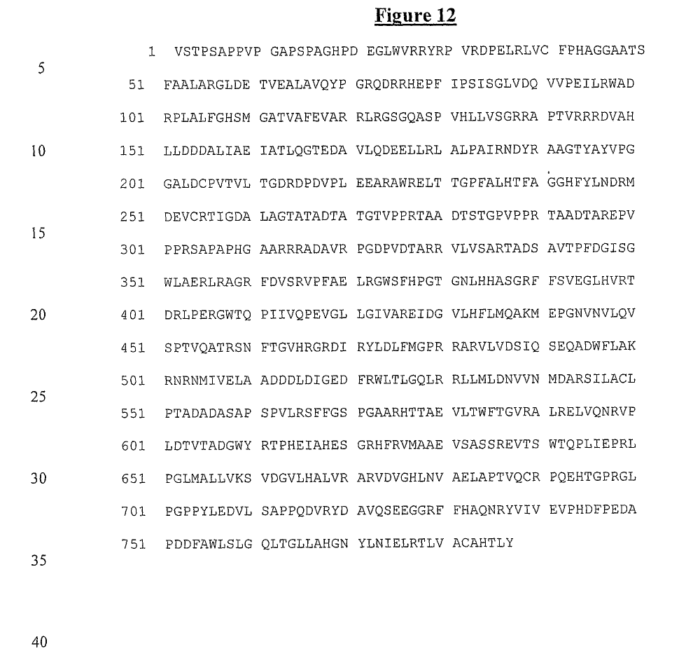

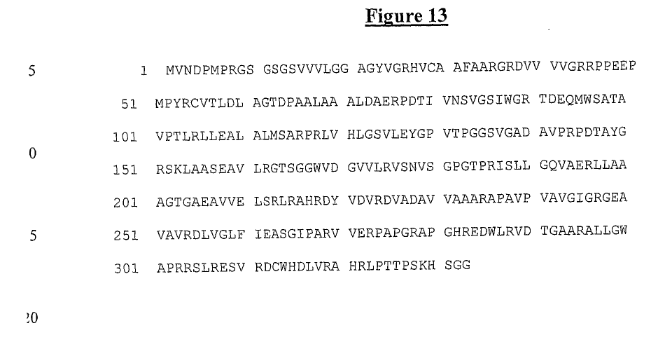

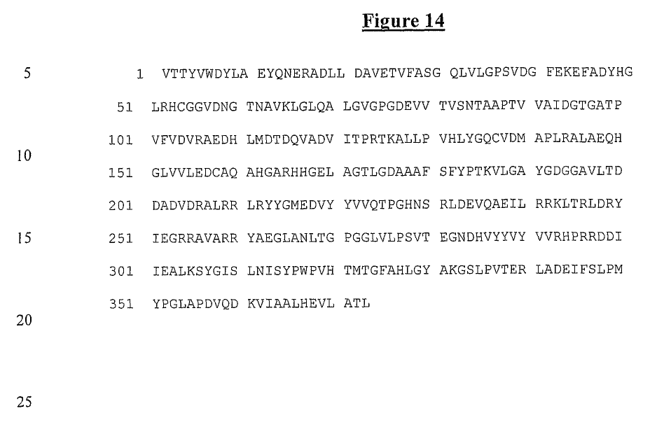

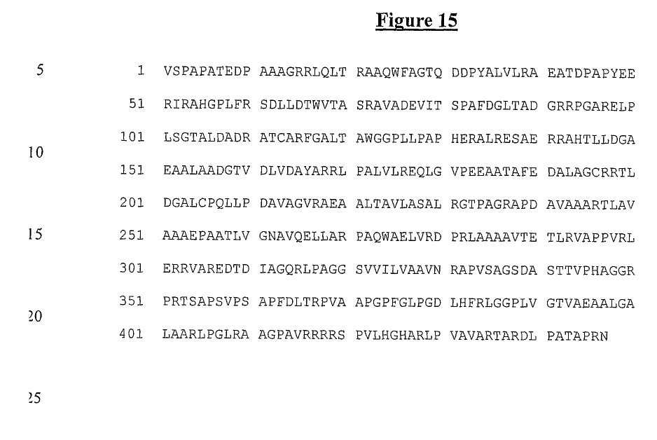

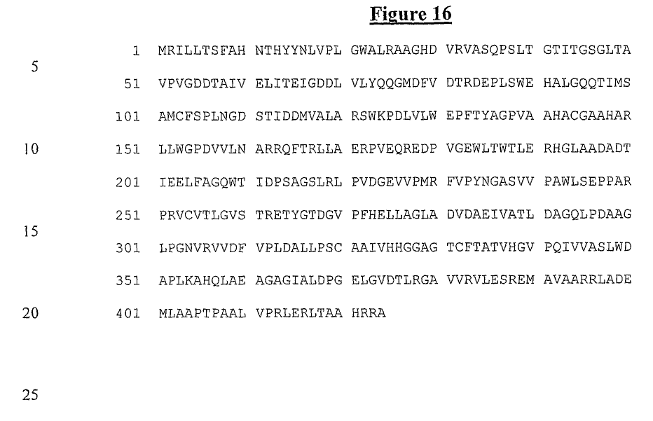

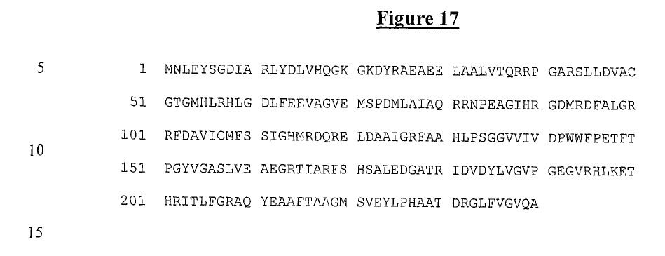

TABLE I
Sequence information for the angolosamine biosynthetic genes
included in the gene cassettes

| Gene (named | | |
|---|---|---|
| according | | |
| to tyl | | Corresponding polypeptide |
| equivalent) | Bases in Figure | Figure number |
| orf2 (angAI) | 14847-15731c from FIG. 8 | FIG. 10 (SEQ ID NO: 8) |
| | (SEQ ID NO: 6) | NDP-hexose synthase |
| orf3 | 13779-14774c from FIG. 8 | FIG. 11 (SEQ ID NO: 9) |
| (angAII) | (SEQ ID NO: 6) | NDP-hexose 4,6-dehydratase |
| orf4 | 11306-13666c from FIG. 8 | FIG. 12 (SEQ ID NO: 10) |
| (N-part) | (SEQ ID NO: 6) | typeII thioesterase |
| (C-part) | | NDP-hexose 2,3-dehydratase |
| orf14 | 1162-2160c from FIG. 7 | FIG. 13 (SEQ ID NO: 11) |
| | (SEQ ID NO: 5) | NDP-hexose 4-ketoreductase |
| orf15 (angB) | 33-1151c from FIG. 7 | FIG. 14 (SEQ ID NO: 12) |
| | (SEQ ID NO: 5) | NDP-hexoseaminotransferase |
| orf1* | 59800-61140 from FIG. 9 | FIG. 15 (SEQ ID NO: 13) |
| (angMIII) | (SEQ ID NO: 7) | Hypothetical NDP hexose 3,4 |
| | | isomerase |
| orf2* | 61159-62430 from FIG. 9 | FIG. 16 (SEQ ID NO: 14) |
| (angMII) | (SEQ ID NO: 7) | angolosaminyl glycosyl |
| | | transferase |
| orf3* | 62452-63171 from FIG. 9 | FIG. 17 (SEQ ID NO: 15) |
| (angMI) | (SEQ ID NO: 7) | N,N-dimethyl transferase |

Note:
c indicates that the gene is encoded by the complement DNA strand
potential functions of the predicted polypeptides (SEQ ID No. 8 to 15) were obtained from the NCBI database using a BLAST search.

### EXAMPLE 1

### Bioconversion of 3-O-mycarosyl erythronolide B to 5-O-dedesosaminyl-5-O-mycaminosyl erythromycins using gene cassette pSG144tylAItylAIItylMIIItylBtyIIatylMIeryCIII

### Isolation of pSG143

Plasmid pSG142 (Gaisser et al., 2000) was digested with XbaI and a fill-in reaction was performed using standard protocols. The DNA was re-ligated and used to transform E. coli DH10B. Construct pSG143 was isolated and the removal of the XbaI site was confirmed by sequence analysis.

### Isolation of pUC18eryBVcas

The gene eryBV was amplified by PCR using the primers casOleG21 (WO01/79520) and 7966 5′-GGGGAATTCAGATCTGGTCTAGAGGTCAGCCGGCGTGGCGGCGCGTGAGTTCCTCCAGTCGC GGGACGATCT -3′ (SEQ ID NO: 16) and pSG142 (Gaisser et al., 2000) as template. The PCR fragment was cloned using standard procedures and plasmid pUC18eryBVcas was isolated with an NdeI site overlapping the start codon of eryBV and XbaI and BglII sites (underlined) following the stop codon. The construct was verified by sequence analysis.

### Isolation of Vector pSGLit1

The isolation of this vector is described in PCT/GB03/003230.

### Isolation of pSGLit1eryCIII

Plasmid pSGCIII (WO01/79520) was digested with NdeI/BglII and the insert fragment was isolated and ligated with the NdeIBglII treated vector fragment of pSGLit1. The ligation was used to transform E. coli ET12567 and plasmid pSGLit1 eryCIII was isolated using standard procedures. The construct was confirmed using restriction digests and sequence analysis. This cloning strategy allows the introduction of a his-tag C-terminal of EryCIII.

### Isolation of pSGLit1 tylMII

Plasmid pSGTYLM2 (WO01/7952) was digested with NdeI/BglII and the insert fragment was isolated and ligated with the NdeI/BglII treated vector fragment of pSGLit1. The ligation was used to transform E. coli ET12567 and plasmid pSGLit1tylMII was isolated using standard procedures. The construct was confirmed using restriction digests and sequence analysis. This cloning strategy allows the introduction of a his-tag C-terminal of TylMII.

### Isolation of pSG144

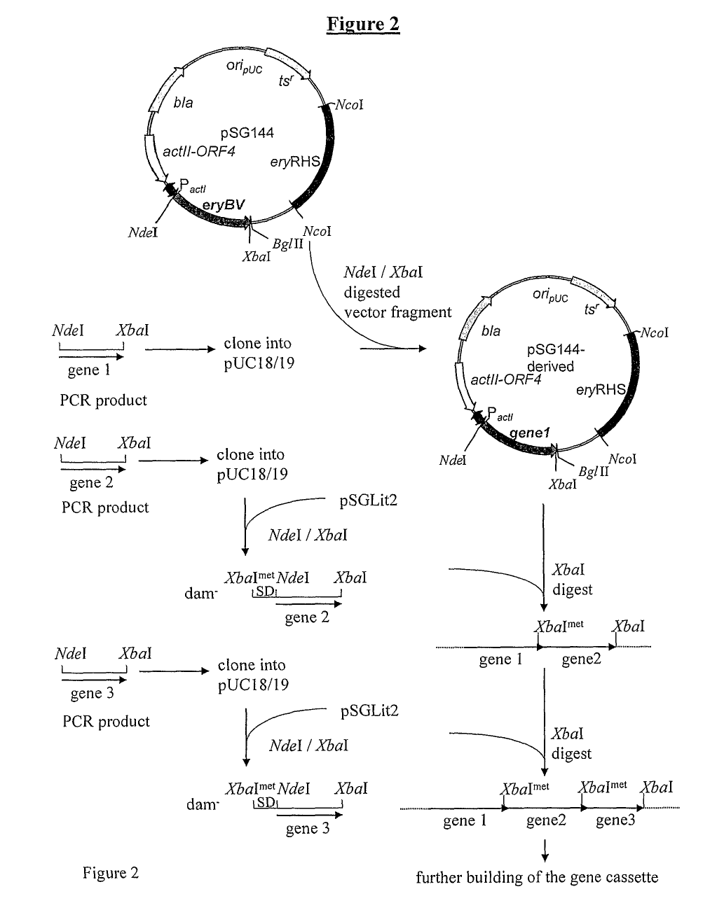

Plasmid pSGLit1 was isolated and digested with NdeI/BglII and an approximately 1.3 kb insert was isolated. Plasmid pSG143 was digested with NdeI/BglII, the vector band was isolated and ligated with the approximately 1.3 kb band from pSGLit1 followed by transformation of E. coli DH10B. Plasmid pSG144 (FIG. 2) was isolated and the construct was verified by DNA sequence analysis. This vector allows the assembly of gene cassettes directly in an expression vector (FIG. 2) without prior assembly in pUC-derived vectors (WO 01/79520) in analogy to PCT/GB03/003230 using vector pSG144 instead of pSGset1. Plasmid pSG144 differs from pSG142 in that the XbaI site between the thiostrepton resistance gene and the eryRHS has been deleted and the his- tag at the end of eryBV has been removed from pSG142 and replaced in pSG144 with an XbaI site at the end of eryBV. This is to facilitate direct cloning of genes to replace eryBV and then build up the cassette.

### Isolation of pSG144eryCIII

EryCIII was amplified by PCR reaction using standard protocols, with primers casOleG21 (WO 01/79520) and caseryCIII2 (WO 01/79520) and plasmid pSGCIII (Gaisser et al., 2000) as template. The approximately 1.3 kb PCR product was isolated and cloned into pUC18 using standard techniques. Plasmid pUCCIIIcass was isolated and the sequence was verified. The insert fragment of plasmid pUCCIIIcass was isolated after NdeI/XbaI digestion and ligated with the NdeI/XbaI digested vector fragment of pSG144. After the transformation of E. coli DH10B plasmid pSG144eryCIII was isolated using standard techniques.

### Isolation of pUC19tylAI

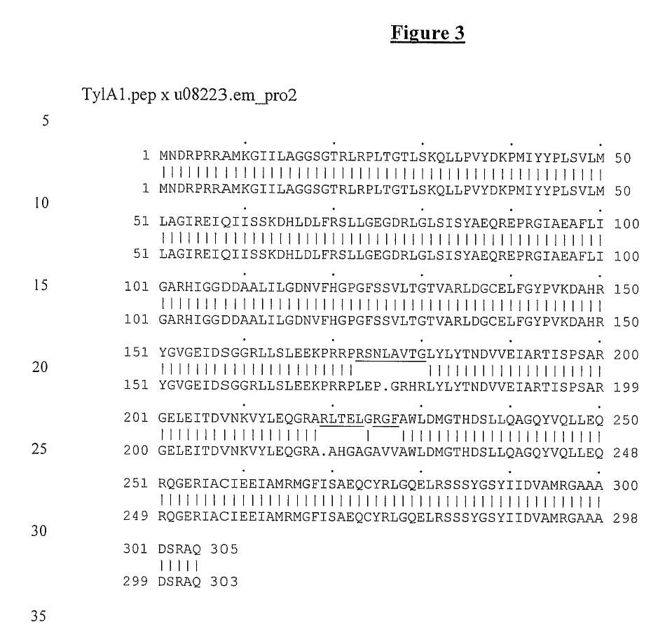

Primers BIOSG34 5′-GGGCATATGAACGACCGTCCCCGCCGCGCCATGAAGGG-3′ (SEQ ID NO: 17) and 5′-CCCCTCTAGAGGTCACTGTGCCCGGCTGTCGGCGGCGGCCCCGCGCATGG-3′ (SEQ ID NO: 18) were used with genomic DNA of Streptomyces fradiae as template to amplify tylAI. The amplified product was cloned using standard protocols and plasmid pUC19tylAI was isolated. The insert was verified by DNA sequence analysis. Differences to the published sequence are shown in FIG. 3.

### Isolation of pSGLit2

Plasmid Litmus 28 was digested with SpeI/XbaI and the vector fragment was isolated. Plasmid pSGLit1 (dam⁻) was digested with XbaI and the insert band was isolated and ligated with the SpeI/XbaI digested vector fragment of Litmus 28 followed by the transformation of E. coli DH10B using standard techniques. Plasmid pSGLit2 was isolated and the construct was verified by restriction digest and sequence analysis. This plasmid can be used to add a 5′ region containing an xbaI site sensitive to Dam methylation and a Shine Dalgarno region thus converting genes which were originally cloned with an NdeI site overlapping the start codon and an bal site 3′ of the stop codon for the assembly of gene cassettes. This conversion includes the transformation of the ligations into E. coli ET12567 followed by the isolation of darn DNA and xbaI digests. Examples for this strategy are outlined below.

### Isolation of pSGLit2tylAI

Plasmid pSGLit2 and pUC19tylAI were digested with NdeI/XbaI and the insert band of pUC19tylAI and the vector band of pSGLit2 were isolated, ligated and used to transform E. coli ET12567. Plasmid pSGLit2tylAI (darn) was isolated.

### Isolation of pUC19tylAII

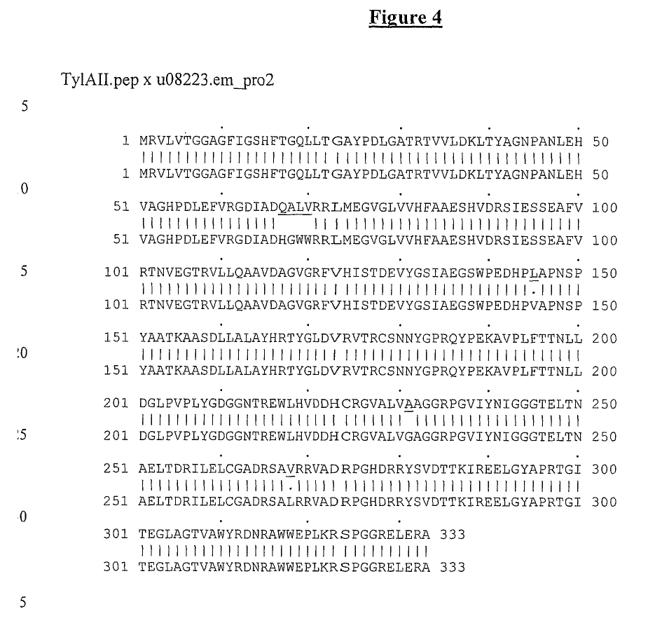

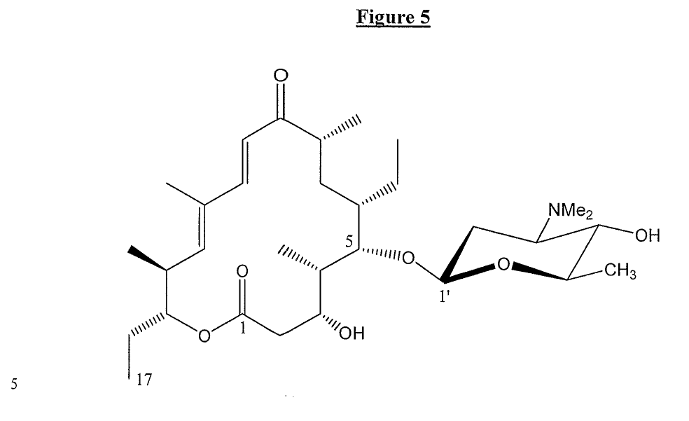

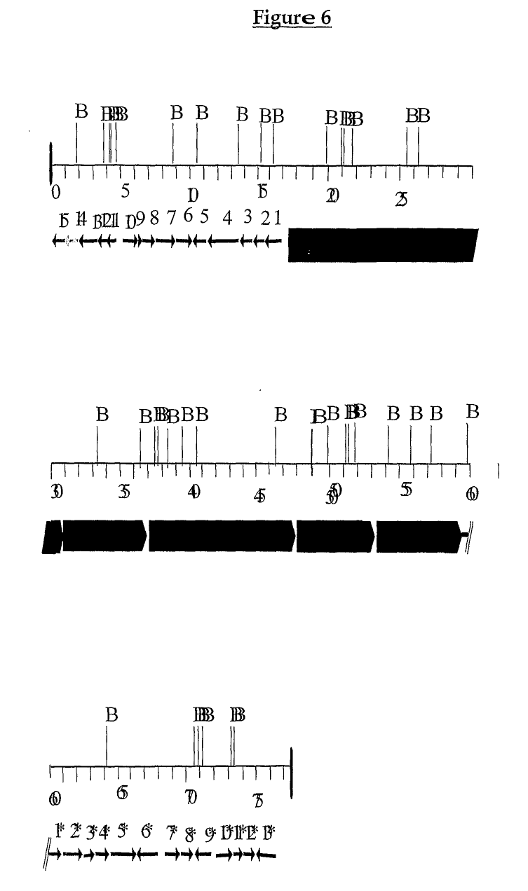

Primers 5′-CCCCTCTAGAGGTCTAGCGCGCTCCAGTTCCCTGCCGCCCGGGGACCGC TTG-3′ (SEQ ID NO: 19) and 5′-GGGTCTAGATCGATTAATTAAGGAGGACATTCATGCGCGT CCTGGTGACCGGAGGTGCGGGCTTCATCGGCTCGCACTTCA-3′ (SEQ ID NO: 20) and genomic DNA of Streptomyces fradiae as template were used for a PCR reaction applying standard protocols to ampIlify tylAII. The approximately 1 kb sized DNA fragment was isolated and cloned into SmaI-cut pUC19 using standard techniques. The DNA sequencing of this construct revealed that 12 nucleotides at the 5′ end had been removed possibly by an exonuclease activity present in the PCR reaction. The comparison of the amino acid sequence of the cloned fragment compared to the published sequence is shown in FIG. 4.

### Isolation of pSGLit2 tylAII

To add the missing 5′-nucleotides, pSGLit2 was digested with PacI/XbaI and the vector fragment was isolated and ligated with the PacI/AbaI digested insert fragment of pUC19tyl4II. The ligated DNA was used to transform E. coli ET12567 and plasmid pSGLit2tylAII (dam⁻) was isolated.

### Isolation of Plasmid pUC19eryCVI

The eryCVI gene was amplified by PCR using primer BIOSG28 5′-GGGCATATGTACGAGGG CGGGTTCGCCGAGCTTTACGACC-3′(SEQ ID NO: 21) and BIOSG29 5′-GGGGTCTAGAGGTCAT CCGCGCACACCGACGAACAACCCG-3′ (SEQ ID NO: 22) and plasmid pNCO62 (Gaisser et al., 1997) as a template. The PCR product was cloned into Smal digested pUC19 using standard techniques and plasmid pUC19eryCVI was isolated and verified by sequence analysis.

### Isolation of Plasmid pSGLit2eryCVI

Plasmid pUC19eryCVI was digested with NdellXbaI and ligated with the NdeIlXbaI digested vector fragment of pSGLit2 followed by transformation of E. coli ET12567. Plasmid pSGLit2eryCVI (dam⁻) was isolated.

### Isolation of Plasmid pSG144tylAI

Plasmid pSG144 and pUC19tylAI were digested with NdeI/XbaI and the insert band of pUC I 9tylAI and the vector band of pSG144 were isolated, ligated and used to transform E. coli DHI10B. Plasmid pSG144tylAI was isolated using standard protocols.

### Isolation of Plasmid pSG144tylAltylAII

Plasmid pSGLit2tylAII (dam⁻) was digested with XbaI and ligated with XbaI digested plasmid pSG144tylAI. The ligation was used to transform E. coli DH10B and plasmid pSG144tylAItylAII was isolated and verified using standard protocols.

### Isolation of Plasmid pSGLit2tylMIII

Plasmid pUC18tylM3 (Isolation described in WO01/79520) was digested with NdeI/XbaI and the insert band and the vector band of NdeIIAbaI digested pSGLit2 were isolated, ligated and used to transform E. coli ET12567. Plasmid pSGLit2tylMIII (dam⁻) was isolated using standard protocols. The construct was verified using restriction digests and sequence analysis.

### Isolation of Plasmid pSG144tylAItylAIItylMII

Plasmid pSGLit2tylMIII (dam⁻) was digested with XbaI and the insert band was ligated with XbaI digested plasmid pSG144tylAltylAII. The ligation was used to transform E. coli DH10B and plasmid pSG144tylAItylAItylMIII no36 was isolated using standard protocols. The construct was verified using restriction digests and sequence analysis.

### Isolation of Plasmid pSGLit2tylB

Plasmid pUC18tylB (Isolation described in WO01/79520) was digested with PacI/XbaI and the insert band and the vector band of PacI/XbaI digested pSGLit2 were isolated, ligated and used to transform E. coli ET12567. Plasmid pSGLit2tylB nol (dam⁻) was isolated using standard protocols.

### Isolation of plasmid pSG144tylAItylAJItylMIIItylB

Plasmid pSGLit2tylB (dam⁻) was digested with XbaI and the insert band was ligated with XbaI digested plasmid pSG144tylAItylAItylMIII. The ligation was used to transform E. coli DH10B and plasmid pSG144tylAItylAIItylMIIItylB no5 was isolated using standard protocols and verified by restriction digests and sequence analysis.

### Isolation of Plasmid pUC18tylIa

Primers BIOSG 88 5′-GGGCATATGGCGGCGAGCACTACGACGGAGGGGAATGT-3′ (SEQ ID NO: 23) and BIOSG 89 5′-GGGTCTAGAGGTCACGGGTGGCTCCTGCCGGCCCTCAG-3′ (SEQ ID NO: 24) were used to amplify tylIa using a plasmid carrying the tyl region (accession number u08223.em_pro2) comprising ORF1 (cytochrome P450) to the end of ORF2 (TyIB) as a template. Plasmid pUCtyIa nol was isolated using standard procedures and the construct was verified using sequence analysis.

### Isolation of Plasmid pSGLit2tylIa

Plasmid pUCtylIa nol was digested with NdeI/XbaI and the insert band and the vector band of NdelIXbaI digested pSGLit2 were isolated, ligated and used to transform E. coli ET12567. Plasmid pSGLit2tylIa no 54 (dam⁻) was isolated using standard protocols. The construct was verified using sequence analysis.

### Isolation ofplasmidpSG144tylAItylAItylMIIItylBtylIa

Plasmid pSGLit2tylIa (dam⁻) was digested with XbaI and the insert band was ligated with XbaI digested plasmid pSG144tylAItylAIItylMIIItylB. The ligation was used to transform E coli DH10B and plasmid pSG144tylAItylAIItylMIIItylBtylIa no3 was isolated using standard protocols and verified by restriction digests and sequence analysis.

### Isolation of Plasmid pSGLit1 tylMIeryCIII

Plasmid pUCtylMI (Isolation described in WO01/79520) was PacI digested and the insert was ligated with the PacI digested vector fragment of pSGLitl eryCIII using standard procedures. Plasmid

pSGL it1tylMIeryCIII no20 was isolated and the orientation was confirmed by restriction digests and sequence analysis.

### Isolation of Gene Cassette pSG144tylAltylAIItylMIIItylBtyIIatylMIeryCIII

Plasmid pSGLit1tylMIeryCIII no20 was digested with XbaI/BglII and the insert band was isolated and ligated with the XbaI/BglII digested vector fragment of plasmid pSG144tylAltylAIItylMIIItylBtylIa no3. Plasmid pSG144tylAItylAIItylMIIItylBtyl1atylMIeryCIII was isolated using standard procedures and the construct was confirmed using restriction digests and sequence analysis. Plasmid preparations were used to transform S. eryth7raea mutant strains with standard procedures.

### Isolation of Plasmid pSGKC1

To prevent the conversion of the substrate 3-O-mycarosyl erythronolide B to 3,5-di-O-mycarosyl erythronolide B a further chromosomal mutation was introduced into S. erythraea SGQ2 (Isolation described in WO 01/79520) to prevent the biosynthesis of L-mycarose in the strain background. Plasmid pSGKCI was isolated by cloning the approximately 0.7 kb DNA fragment of the eryBVIgene by using PCR amplification with cosmid2 or plasmid pGG1 (WO01/79520) as a template and with the primers 646 5′-CATCGTCAAGGAGTTCGACGGT-3′ (SEQ ID NO: 25) and 874 5′-GCCAGCTCGGCGACGTCC ATC-3′ (SEQ ID NO: 26) using standard protocols. Cosmid 2 containing the right hand site of the ery-cluster was isolated from an existing cosmid library (Gaisser et al., 1997) by screening with eryBVas a probe using standard techniques. The amplified DNA fragment was isolated and cloned into EcoRV digested pKC1132 (Bierman et al., 1992) using standard methods. The ligated DNA was used to transform E. coli DH10B and plasmid pSGKCl was isolated using standard molecular biological techniques. The construct was verified by DNA sequence analysis.

Isolation of S. erythraea Q42/1 (Biot-2166) Plasmid pSGKC1 was used to transform S. erythraea SGQ2 using standard techniques followed by selection with apramycin. Thiostrepton/apramycin resistant transformant S. erythraea Q42/1 was isolated.

### Bioconversion using S. erythraea Q42/1 pSG144tylAItylAIItylMIIItylBtyl1atylMIeryCIII

Bioconversion assays using 3-O-mycarosyl erythronolide B are carried out as described in General Methods. Improved levels of mycaminosyl erythromycin A are detected in bioconversion assays using S. erythraea Q42/1 pSG144tylAItylAIItylMIIItylBtyl1atylMIeryCIII compared to bioconversion levels previously observed (WO01/79520).

### EXAMPLE 2

### Isolation of Mycaminosyl Tylactone using Gene Cassette pSG144tylAItylAIItylMIIItylBtylIatylMItylMII

### Isolation of Plasmid pSGLit1tylMItylMII

Plasmid pUCtylMI (Isolation described in WO1/79520) was PacI digested and the insert was ligated with the PacI digested vector fragment of pSGLit1 tylMII using standard procedures. Plasmid pSGLit1tylMItylMII no16 was isolated and the construct was confirmed by restriction digests and sequence analysis.

### Isolation of Plasmid pSG144tylAItylAIItylMIIItylBtylIatylMItylMII

Plasmid pSGLit1tylMItylMII no16 was digested with XbaI/BglII and the insert band was isolated and ligated with the XbaI/BglII digested vector fragment of plasmid pSG144tylAItylAItylMIItylBtylIa no3. Plasmid pSG144tylAItylAIItylMIIItylBtyl1atylMItylMII was isolated using standard procedures and the construct was confirmed using restriction digests and sequence analysis. The plasmid was isolated and used for transformation of S. erythraea mutant strains using standard protocols.

### Bioconversion using Gene Cassette pSG144tylAItylAIItylMIIItylBtyl1atylMItylMII

The conversion of fed tylactone to mycaminosyl tylactone was assessed in bioconversion assays using S. erythraea Q42/1pSG144tylAItylAIItylMIIItylBtyl1atylMItylMII. Bioconversion assays were carried out using standard protocols. The analysis of the culture showed the major ion to be 568.8 [M+H]⁺ consistent with the presence of mycaminosyl tylactone. Fragmentation of this ion gave a daughter ion of m/z 174, as expected for protonated mycaminose. No tylactone was detected during the analysis of the culture extracts, indicating that the bioconversion of the fed tylactone was complete.

Recently, a homologue of TyIIa was identified in the biosynthetic pathway of dTDP-3-acetamido-3,6-dideoxy-alpha-D-galactose in Aneurinibacillus therm oaerophilus L420-91^{T}* (Pfoestl et al., 2003) and the function was postulated as a novel type of isomerase capable of synthesizing dTDP-6-deoxy-D-xylohex-3-ulose from dTDP-6-deoxy-D-xylohex-4-ulose.

### EXAMPLE 3

### Bioconversion of 3-O-mycarosyl erythronolide B to 5-O-dedesosaminyl-5-O-mycaminosyl erythromycins using gene cassette pSG1448/27/95/21/44/193/6eryCIII

(pSG144angAIangAIIorf14angMIIIangBangMIeryCIII).

### Cloning of angMIII by Isolating Plasmid Lit1/4

The gene angMIII was amplified by PCR using the primers BIOSG61 5′-GGGCATATGAGCCCCGCACCCGCCACCGAGGACCC-3′ (SEQ ID NO: 27) and BIOSG62 5′-GGTCTAGAGGTCAGTTCCGCGGTGCGGTGGCGGGCAGGTCAC -3′ (SEQ ID NO: 28). Cosmid5B2 containing a fragment of the angolamycin biosynthetic pathway was used as template. The 1.4 kb PCR fragment (PCR no1) was cloned using standard procedures and EcoRV digested plasmid Litmus28. Plasmid Lit1/4 was isolated with an NdeI site overlapping the start codon of angMIII and an XbaI site following the stop codon. The construct was verified by sequence analysis.

### Isolation of Plasmid pSGLit21/4

Plasmid Lit1/4 was digested with NdeI/XbaI and the about 1.4 kb fragment was isolated and ligated to NdeI/XbaI digested DNA of pSGLit2. The ligation was used to transform E. coli ET12567 and plasmid pSGLt21/4 no 7 (dam⁻) was isolated. This construct was digested with XbaI and used for othe construction of gene cassettes.

### Cloning of angMII by Isolating Plasmid Lit2/8

The gene angMII was amplified by PCR using the primers BIOSG63 5′-GGGCATATGCGTATC CTGCTGACGTCGTTCGCGCACAACAC-3′(SEQ ID NO: 29) and BIOSG64 5′-GGTCTAGAGGTCA GGCGCGGCGGTGCGCGGCGGTGAGGCGTTCG-3′ (SEQ ID NO: 30) and cosmid5B2 containing a fragment of the angolamycin biosynthetic pathway was used as template. The 1.3 kb PCR fragment (PCR no2) was cloned using standard procedures and EcoRV digested plasmid Litmus28. Plasmid Lit2/8 was isolated with an NdeI site overlapping the start cocon of angMII and an XbaI site following the stop codon. The construct was verified by sequence analysis.

### Cloning of angMII by Isolating Plasmid pLitangMII(BglII)

The gene angMII was amplified by PCR using primers BIOSG63 5′-GGGCATATGCGTATCCT GCTGACGTCGTTCGCGCACAACAC-3′ (SEQ ID NO: 29) and BIOSG80 5′-GGAGATCTGGCGCG GCGGTGCGCGGCGGTGAGGCGTTCG-3′ (SEQ ID NO: 31) and cosmid5B2 containing a fragment of the angolamycin biosynthetic pathway as template. The 1.3 kb PCR fragment was cloned using standard procedures and EcoRV digested plasmid Litmus28. Plasmid LitangMII(BGlII)no8 was isolated with an NdeI site overlapping the start codon of angMII and a BglII site instead of a stop codon thus allowing the addition of a his-tag. The construct was verified by sequence analysis.

### Isolation of Plasmid pSGLit1angMII

Plasmid LitangMII(BgIII) was digested with NdeI/BglII and ligated with the NdeI/BglII digested vector fragment of pSGLit1. The ligation was used to transform E. coli ET12567 and plasmid psGLit1angMII (dam⁻) was isolated using standard procedures.

### Cloning of angMI by Isolating Plasmid Lit3/6

The gene angMI was amplified by PCR using the primers BIOSG65 5′-GGGCATATGAAC CTCGAATACAGCGGCGACATCGCCCGGTTG -3′ (SEQ ID NO: 32) and BIOSG66 5′-GGTCTAGAGGTCAGGCCTGGACGCCGACGAAGAGTCCGCGGTCG-3′ (SEQ ID NO: 33) and cosmid5B2 containing a fragment of the angolamycin biosynthetic pathway was used as template. The 0.75 kb PCR fragment (PCR no3) was cloned using standard procedures and EcoRV digested plasmid Litmus28. Plasmid Lit3/6 was isolated with an NdeI site overlapping the start codon of angMI and an XbaI site following the stop codon. The construct was verified by sequence analysis.

### Isolation of Plasmid pSGlit23/6 no8

Plasmid Lit3/6 was digested with NdeI/XbaI and the about 0.8 kb fragment was isolated and ligated to NdeI/XbaI digested DNA of pSGLit2. The ligation was used to transform E. coli ET12567 and plasmid pSGLit23/6 no8 (dam⁻) was isolated. This construct was digested with XbaI and the isolated about 1 kb fragment was used for the assembly of gene cassettes.

### Cloning of angB by Isolating Plasmid Lit4/19

The gene angB was amplified by PCR using the primers BIOSG67 5′-GGGCATATGACTACCT ACGTCTGGGACTACCTGGCGG -3′ (SEQ ID NO: 34) and BIOSG68 5′-GGTCTAGAGGTCAGAGC GTGGCCAGTACCTCGTGCAGGGC-3′ (SEQ ID NO: 35) and cosmid4H2 containing a fragment of the angolamycin biosynthetic pathway was used as template. The 1.2 kb PCR fragment (PCR no4) was cloned using standard procedures and EcoRV digested plasmid Litmus28. Plasmid Lit4/19 was isolated with an NdeI site overlapping the start codon of angB and an XbaI site following the stop codon. The construct was verified by sequence analysis.

### Isolation of Plasmid pSGlit24/19

Plasmid Lit4/19 was digested with NdeI/XbaI and the 1.2 kb fragment was isolated and ligated into NdeI/XbaI digested DNA of pSGLit2. The ligation was used to transform E. coli ET12567 and plasmid pSGLit24/19 no24 (dam⁻) was isolated. This construct was digested with XbaI and the isolated 1.2 kb fragment was used for the assembly of gene cassettes.

### Cloning of orf14 by Isolating Plasmid Lit5/2

The gene orf14 was amplified by PCR using the primers BIOSG69 5′-GGGCATATGGTGAA CGATCCGATGCCGCGCGGCAGTGGCAG-3′ (SEQ ID NO: 36) and BIOSG70 5′-GGTCTAGAGGT CAACCTCCAGAGTGTTTCGATGGGGTGGTGGG-3′ (SEQ ID NO: 37) and cosmid4H2 containing a fragment of the angolamycin biosynthetic pathway was used as template. The 1.0 kb PCR fragment (PCR no5) was cloned using standard procedures and EcoRV digested plasmid Litmus28. Plasmid Lit5/2 was isolated with an NdeI site overlapping the start codon of ORF14 and an XbaI site following the stop codon. The construct was verified by sequence analysis.

### Isolation of Plasmid pSGlit25/2 no24

Plasmid Lit5/2 was digested with NdeI/XbaI and the approximately 1 kb fragment was isolated and ligated to NdeI/Xbal digested DNA of pSGLit2. The ligation was used to transform E. coli ET12567 and plasmid pSGLit25/2 no24 (dam⁻) was isolated. This construct was digested with XbaI, the about 1 kb fragment isolated and used for the assembly of gene cassettes.

### Isolation of Plasmid pSGlit27/9 no15

Plasmid Lit7/9 was digested with NdeI/XbaI and the approximately 1 kb fragment was isolated and ligated to NdeI/XbaI digested DNA of pSGLit2. The ligation was used to transform E. coli ET12567 and plasmid pSGLit27/9 no15 (dam⁻) was isolated. This construct was digested with XbaI and the isolated 1 kb fragment was used for the assembly of gene cassettes.

### Cloning of angAI (orj2) by Isolating Plasmid Lit8/2

The gene angAI was amplified by PCR using the primers BIOSG73 5′-GGGCATATGAAGGGC ATCATCCTGGCGGGCGGCAGCGGC-3′ (SEQ ID NO: 38) and BIOSG74 5′-GGTCTAGAGGTCAT GCGGCCGGTCCGGACATGAGGGTCTCCGCCAC-3′ (SEQ ID NO: 39) and cosmid4H2 containing a fragment of the angolamycin biosynthetic pathway was used as template. The around 1.0 kb PCR fragment (PCR no8) was cloned using standard procedures and EcoRV digested plasmid Litmus28. Plasmid Lit8/2 was isolated with an NdeI site overlapping the start codon of angAI and an XbaI site following the stop codon. The construct was verified by sequence analysis.

### Cloning of angAII (orf3) by isolating plasmid Lit7/9

The gene angaII was amplified by PCR using the primers BIOSG71 5′-GGGCATATGCGGCTG CTGGTCACCGGAGGTGCGGGC-3′ (SEQ ID NO: 40) and BIOSG72 5′-GGTCTAGAGGTCAGTCG GTGCGCCGGGCCTCCTGCG-3′ (SEQ ID NO: 41) and cosmid4H2 containing a fragment of the angolamycin biosynthetic pathway was used as template. The 1.0 kb PCR fragment was cloned using standard procedures and EcoRV digested plasmid Litmus28. Plasmid Lit7/9 was isolated with an NdeI site overlapping the start codon of angAII and an XbaI site following the stop codon. The construct was verified by sequence analysis.

### Isolation of Plasmid pSGlit28/2 no18 (pSGLit2angAI)

Plasmid Lit8/2 was digested with NdellXbaI and the 1 kb fragment was isolated and ligated to NdeI/XbaI digested DNA of pSGLit2. The ligation was used to transform E. coli ET12567 and plasmid pSGLit28/2 no18 (dam⁻) was isolated.

### Isolation of Plasmid pSG1448/2 (pSG144angAI)

Plasmid Lit8/2 was digested with NdeI/XbaI and the approximately 1 kb fragment was isolated and ligated with NdeI/XbaI digested DNA of pSG144. The ligation was used to transform E. coli DH10B and plasmid pSG1448/2 (dam⁻) (pSG144angAI) was isolated using standard procedures. This construct was verified with restriction digests and sequence analysis.

### Isolation of Plasmid pSG1448/27/9 (pSG144angAIangAII)

Plasmid pSGLit27/9 (isolated from E. coli ET12567) was digested with XaI and the 1 kb fragment was isolated and ligated with the XbaI digested vector fragment of pSG1448/2 (pSG144angAI).

The ligation was used to transform E. coli DH10B and plasmid pSG1448/27/9 (pSG144angAIangAII) was isolated using standard protocols. The construct was verified with restriction digests and sequence analysis.

### Isolation of Plasmid pSG1448/27/91/4 (pSG144angAIangAIIangMIII)

Plasmid pSGLit21/4 (isolated from E. coli ET12567) was digested with XbaI and the 1.4 kb fragment was isolated and ligated with the XbaI digested vector fragment of pSG1448/27/9 (pSG144angAIangAII). The ligation was used to transform E. coli DH10B and plasmid pSG1448/27/91/4 (pSG144angAIanggAIangMIII) was isolated using standard protocols. The construct was verified with restriction digests and sequence analysis.

### Isolation of Plasmid pSG1448/27/91/44/19 (pSG144angAIangAIIangMIIIangB)

Plasmid pSGLit24/19 (isolated from E. coli ET12567) was digested with XbaI and the about 1.2 kb fragment was isolated and ligated with the XbaI digested vector fragment of pSG1448/27/91/4 (pSG144angAIangAIIangMIII). The ligation was used to transform E. coli DH10B and plasmid pSG144/27/91/44/19 (pSG144angAIangAIIangMIIIangB) was isolated using standard protocols. The construct was verified with restriction digests and sequence analysis.

### Isolation of Plasmid pSG1448/27/91/44/193/6 (pSG144angAIangAIIangMIIIangBangMI)

Plasmid pSGLit23/6 (isolated from E. coli ET12567) was digested with XbaI and the about 0.8 kb fragment was isolated and ligated with the XbaI digested vector fragment of pSG1448/27/91/44/19 (pSG144angAIang4AIIangMIIIangB). The ligation was used to transform E. coli DH10B and plasmid pSG1448/27/91/44/193/6 (pSG144angAIangAIIangMIIIangBangMI) was isolated using standard protocols. The construct was verified with restriction digests and sequence analysis.

### Isolation of Plasmid pSG1448/27/91/44/193/6eryCIII (pSG144ang/AIang/AIIang)MIIIangBangMIeryCIII)

Plasmid pSGLit1eryCIII (isolated from E. coli ET12567) was digested with XbaI/BglII and the about 1.2 kb fragment was isolated and ligated with the XbaI digested and partially BglII digested vector fragment of pSG1448/27/91/44/193/6 (pSG144angAIangAIIangMIIIangBangMI). The BglII partial digest Was necessary due to the presence of a BglII site in angB. The ligation was used to transform E. coli DH10B and plasmid pSG1448/27/91/44/193/6eryCIII no9 (pSG144angAIangAIIangMIIIangBangMIeryCIII) was isolated using standard protocols. The construct was verified with restriction digests and sequence analysis. EryCIII carries a his-tag fusion at the end.

### Bioconversion of 3-O-mycarosyl erythronolide B to 5-O-dedesosaminyl-5-O-mycaminosyl erythromycin A using S. erythraea q42/1pSG1448/727/91/44/193/6eryCIII no9

(pSG144angAIangAIIangMIIIangBangMIeryCIII)

The S. erythraea strain Q42/1pSG1448/27/91/44/193/6eryCIII was grown and bioconversions with fed 3-O-mycarosyl erthronolide B were performed as described in the General Methods. The cultures were analysed and a small amount of a compound with m/z 750 was detected consistent with the presence of 5-O-dedesosaminyl-5-O-mycaminosyl erythromycin A.

### Isolation of Plasmid pSG1448/27/95/2 (pSG144angAIangAIIorf14)

Plasmid pSGLit25/2 (isolated from E. coli ET12567) was digested with XbaI and the about 1 kb fragment was isolated and ligated with the XbaI digested vector fragment of pSG1448/27/9 (pSG144angAIangAII). The ligation was used to transform E. coli DH10B and plasmid pSG1448/27/95/2 (pSG144angAIangAIIorf14) was isolated using standard protocols. The construct was verified with restriction digests and sequence analysis.

### Isolation of Plasmid pSG1448/27/95/21/4 (pSG144angAIangAIIorf14angMIII)

Plasmid pSGLit21/4 (isolated from E. coli ET12567) was digested with XbaI and the 1.4 kb fragment was isolated and ligated with the XbaI digested vector fragment of pSG1448/27/95/2 (pSG144angAIangAIIorf14). The ligation was used to transform E. coli DH10B and plasmid pSG1448/27/95/21/4 (pSG144angAlIangAIIorf14angMIII) was isolated using standard protocols. The construct was verified with restriction digests and sequence analysis.

### Isolation of Plasmid pSG1448/27/95/21/44/19 (pSG144ang/AIangAIIorf14angMIIIangB)

Plasmid pSGLit24/19 (isolated from E. coli ET12567) was digested with XbaI and the 1.2 kb fragment was isolated and ligated with the XbaI digested vector fragment of pSGI448/27/95/21/4 (pSG 144angAIangAIIorf4angMIII). The ligation was used to transform E. coli DH10B and plasmid pSG 1448/27/95/21/44/19 (pSG 144angaIangAIIorf1 4angMIIIangB) was isolated using standard protocols. The construct was verified with restriction digests and sequence analysis.

### Isolation of Plasmid pSG1448/27/95/21/44/193/6eryCIII

(pSG144angAIang/AIIorf14angMIIIangBangMIeryCIII)

Plasmid pSG1448/27/91/44/193/6eryCIII no9 was digested with BglII and the about 2 kb fragment was isolated and ligated with the BglI digested vector fragment of pSG1448/27/95/21/44/19 (pSG144angaIangAIIorf14angMIIIIangB). The ligation was used to transform E. coli DH10B and plasmid pSG 1448/27/95/21/44/193/6eryCIII (pSG144angAIangAIIorf14angMIIIangBangMIeryCIII) was isolated using standard protocols. The construct was verified with restriction digests and sequence analysis. EryCIII carries a his-tag fusion at the end. The construct was used to transform S. erythraea SGQ2 using standard procedures.

### Bioconversion of 3-O-mycarosyl erythronolide B to 5-O-dedesosaminyl-5-O-mycaminosyl erythromycin A

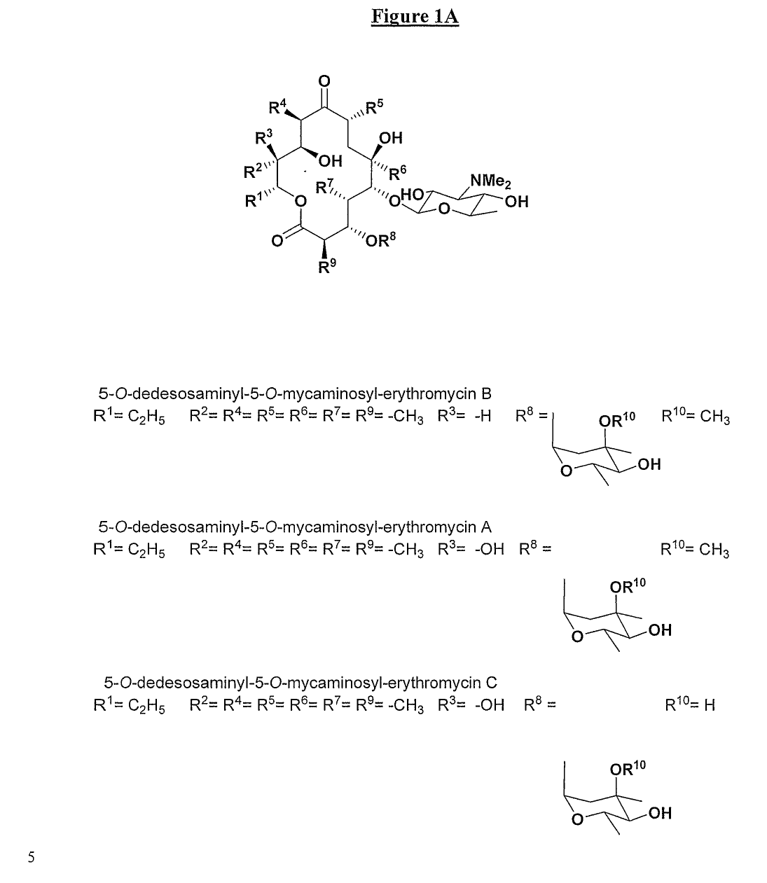

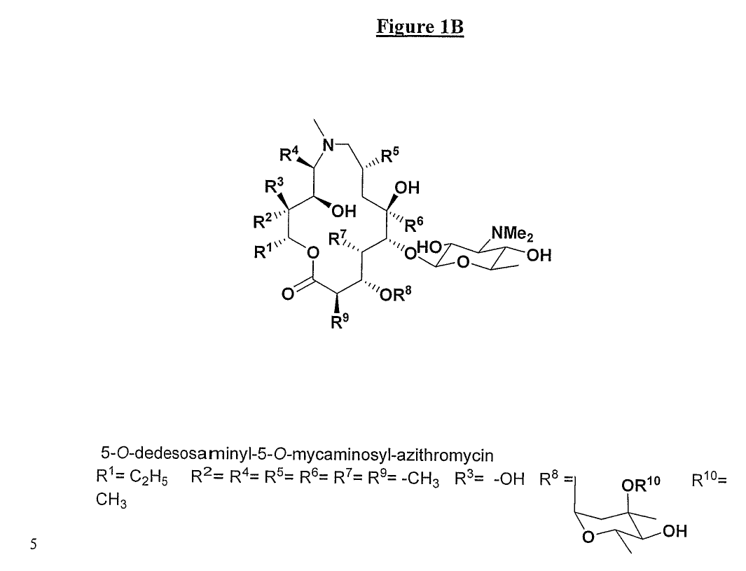

The S. erythraea strain SGQ2pSG1448/27/95/21/44/193/6eryCIII was grown and bioconversions with fed 3-O-mycarosyl erythronolide B were performed as described in the General Methods. The cultures were analysed and improved amounts of a compound with m/z 750 was detected consistent with the presence of 5-O-dedesosaminyl-5-O-mycaminosyl erythromycin A. Similar results were obtained with the S. erythraea strain Q42/1 containing the gene cassette pSG1448/27/95/21/44/193/6eryCIII. 16 mg of the compound with m/z 750 was purified and the structure of 5-O-dedesosaminyl-5-O- inycaminosyl erythromycin A was confirmed by NMR analysis (See Table I and FIG. 1).

TABLE II
¹H and ¹³C NMR data for 5-O-dedesosaminyl-5-O-mycaminosyl
erythromycin A (BC156)

| Position | δ_{H} | Multiplicity | Coupling | δ_{C} |
|---|---|---|---|---|
| 1 | | | | 175.4 |
| 2 | 2.83 | dq | 9.6, 7.1 | 44.9 |
| 3 | 3.91 | dd | 9.7, 1.6 | 80.0 |
| 4 | 2.00 | m | | 39.1 |
| 5 | 3.53 | d | 6.8 | 85.4 |
| 6 | | | | 74.8 |
| 7 | 1.66 | dd | 14.8, 2.2 | 38.5 |
| | 1.82 | dd | 14.8, 11.4 | |
| 8 | 2.69 | dqd | 11.3, 7.0, 2.2 | 44.9 |
| 9 | | | | 221.6 |
| 10 | 3.06 | qd | 6.9, 1.3 | 38.0 |
| 11 | 3.81 | d | 1.3 | 68.9 |
| 12 | | | | 74.6 |
| 13 | 5.04 | dd | 11.0, 2.3 | 76.8^{a} |
| 14 | 1.47 | dqd | 14.3, 11.0, 7.2 | 21.1 |
| | 1.91 | ddq | 14.3, 7.5, 2.2 | |
| 15 | 0.83 | dd | 7.4, 7.4 | 10.6 |
| 16 | 1.18 | d | 7.1 | 16.0 |
| 17 | 1.03 | d | 7.4 | 9.7 |
| 18 | 1.44 | s | | 26.6 |
| 19 | 1.16 | d | 7.0 | 18.3 |
| 20 | 1.14 | d | 7.0 | 12.0 |
| 21 | 1.12 | s | | 16.2 |
| 1′ | 4.87 | d | 4.8 | 96.4 |
| 2′ | 1.55 | dd | 15.2, 4.8 | 34.9 |
| | 2.32 | dd | 15.2, 0.9 | |
| 3′ | | | | 72.8 |
| 4′ | 3.01 | d | 9.3 | 77.8 |
| 5′ | 3.99 | dq | 9.3, 6.2 | 65.6 |
| 6′ | 1.27 | d | 6.2 | 18.5 |
| 7′ | 1.23 | s | | 21.4 |
| 8′ | 3.29 | s | | 49.4 |
| 1″ | 4.43 | d | 7.4 | 103.3 |
| 2″ | 3.56 | dd | 10.5, 7.3 | 71.3 |
| 3″ | 2.48 | dd | 10.3, 10.3 | 70.6 |
| 4″ | 3.09 | dd | 9.9, 9.0 | 70.2 |
| 5″ | 3.31 | dq | 9.0, 6.1 | 72.9 |
| 6″ | 1.29 | d | 6.1 | 18.1 |
| 7″ | 2.58 | s | | 41.7 |

^{a}This carbon was assigned from the HMQC spectrum

### EXAMPLE 4

### Isolation of Mycaminosyl Tylactone

### Isolation of Plasmid pSG1448/27/95/21/44/193/6tylMII

(pSG144angAIangAIIorf14angMIIIangB3/6tylMII)

Plasmid pSG1448/27/91/44/193/6tylMII no9 was digested with BglII and the about 2 kb fragment was isolated and ligated with the BglII digested vector fragment of pSG1448/27/95/21/44/19 (pSG144angAIangAIIorf14angMIIIangB). The ligation was used to transform E. coli DHIOB and plasmid pSG1448/27/95/21/44/193/6tylMII (pSG144angAIangAIIorf14angMIIIangBangMItylMII) was isolated using standard protocols. The construct was verified with restriction digests and sequence analysis. TylMII carries a his-tag fusion at the end.

### Bioconversion of Tylactone to Nycaminosyl Tylactone

The S. erythraea strain Q42/1pSG1448/27/95/21/44/193/6tylMII is grown and bioconversions with fed tylactone is performed as described in the General Methods. The cultures are analysed and a compound with In/z 568 is detected consistent with the presence of mycaminosyl tylactone.

### EXAMPLE 5

### Isolation of 5-O-dedesosaminyl-5-O-angolosaminyl Erythromycins using Gene Cassette pSG1448/27/91/4spnO5/2p4/193/6tylMII by Bioconversion of 3-O-mycarosyl erythronolide B

### Isolation of Plasmid Conv Nol

For the multiple use of promoter sequences in act-controlled gene cassettes a 240 bp fragment was amplified by PCR using the primers BIOSG78 5′-GGGCATATGTGTCCTCCTTAATTAATCGAT GCGTTCGTCC-3′ (SEQ ID NO: 42) and BIOSG79 5′-GGAGATCTGGTCTAGATCGTGTTCCCCTCC CTGCCTCGTGGTCCCTCACGC -3′ (SEQ ID NO: 43) and plasmid pSG142 (Gaisser et al., 2000) as template. The 0.2 kb PCR fragment (PCR no5) was cloned using standard procedures and EcoRV digested plasmid Litmus28. Plasmid conv nol was isolated. The construct was verified by sequence analysis.

### Isolation of pSGLit3relig1

Plasmid conv nol was digested with NdeJ/BglII and the about 0.2 kb fragment was isolated and ligated with the BamHI/NdeI digested vector fragment of pSGLit2. The ligation was used to transform E. coli DH10B and plasmid pSGLit3relig1 was isolated using standard procedures. This construct was verified using restriction digests and sequence analysis.

### Isolation of Plasmid pSGlit34/19

Plasmid Lit4/19 was digested with NdeI/XbaI and the 1.2 kb fragment was isolated and ligated to NdeI/XbaI digested DNA of pSGLit3. The ligation was used to transform E. coli ET12567 and plasmid pSGLit34/19 no23 was isolated. This construct was digested with xbaI and the isolated 1.4 kb fragment was used for the assembly of gene cassettes.

### Cloning of orf4 by Isolating Olasmnid Lit6/4

The gene orf4 was amplified by PCR using the primers BIOSG75 5′-GGGCATATGAGCACCC CTTCCGCACCACCCGTTCCG-3′ (SEQ ID NO: 44) and BIC)SG76 5′-GGTCTAGAGGTCAGTACAG CGTGTGGGCACACGCCACCAG-3′ (SEQ ID NO: 45) and cosmid4H2 containing a fragment of the angolainycin biosynthetic pathway was used as template. The 2.5 kb PCR fragment (PCR no6) was cloned using standard procedures and EcoRV digested plasmid Litmus28. Plasmid Lit6/4 was isolated with an Ndel site overlapping the start codon of orf4 and an XbaI site following the stop codon. The construct was verified by sequence analysis.

### Isolation of Plasmid pSGlit26/4 no9

Plasmid Lit6/4 was digested with NdeI/XbaI and the DNA was isolated and ligated to NdeI/AbaI digested DNA of pSGLit2. The ligation was used to transform E. coli ET12567 and plasmid pSGLit26/4 no9 was isolated. This construct was confirmed by restriction digests and sequence analysis.

### Cloning of spnO by Isolation Plasmid pUC19spnO

The gene spnO from the spinosyn biosynthetic gene cluster of Saccharopolyspoia spinosa was amplified by PCR using the primers BIOSG41 5′-GGGCATATGAGCAGTTCTGTCGAAGCTGAGGC AAGTG-3′ (SEQ ID NO: 46) and BIOSG42 5′-GGTCTAGAGGTCATCGCCCCAACGCCCACAAGCT ATGCA GG-3′ (SEQ ID NO: 47) and genomic DNA of S. spinosa as template. The about 1.5 kb PCR fragment was cloned using standard procedures and SmaI digested plasmid pUC19. Plasmid pUC19spnO no2 was isolated with an NdeI site overlapping the start codon of spnO and an XbaI site following the stop codon. The construct was verified by sequence analysis.

### Isolation of Plasmid pSGlit2spnO no4

Plasmid pUC19spnO was digested with NdeI/XbaI and the 1.5 kb fragment was isolated and ligated to NdeI/XbaI digested DNA of pSGLit2. The ligation was used to transform E. coli ET12567 and plasmid pSGLit2spnO no 4 was isolated using standard procedures. This construct was digested with XbaI and the isolated 1.5 kb fragment was used for the assembly of gene cassettes.

### Isolation of Plasmid pSG1448/27/91/4spnO (pSG144angAIang/AIIangMIIIspnO)

Plasmid pSGLit2spnO no4 (isolated from E. coli ET12567) was digested with XbaI and the 1.5 kb fragment was isolated and ligated with the XbaI digested vector fragment of pSG1448/27/91/4 (pSG144angAIangAIIangMIII). The ligation was used to transform E. coli DH10B and plasmid pSG1448/27/91/4spnO (pSG144angAIangAIIangMIIIspnO) was isolated using standard protocols. The construct was verified with restriction digests and sequence analysis.

### Isolation of Plasmid pSG1448/27/91/4spnO5/2 (pSG144angAIangAIIangMIIIspnOangorf14)

Plasmid pSGLit25/2 no24 (isolated from E. coli ET 12567) was digested with XbaI and the 1 kb fragment was isolated and ligated with the XbaI digested vector fragment of pSG1448/27/91/4spnO (pSG144angaIangAIIangMIIIspnO). The ligation was used to transform E. coli DH10B and plasmid pSG1448/27/91/4spnO5/2 (pSG144angaIangAIIangMIIspnOangorfl4) was isolated using standard protocols. The construct was verified with restriction digests and sequence analysis.

### Isolation of Plasniid pSG1448/27/91/4spnO5/2p4/19 (pSG144angAIangAIIangMIIIspnOangorf14pangB)

Plasmid pSGLit34/19 no23 (isolated from E. coli ET12567) was digested with XbaI and the about 1.4 kb fragment was isolated and ligated with the XbaI digested vector fragment of pSG1448/27/91/4spnO5/2 (pSG144angAIangAIIangMIIIspnOangorf14). The ligation was used to transform E. coli DH10B and plasmid pSG1448/27/91/4spnO5/2p19 (pSG 144angaIangAIIangMIIIspnOangorf14pangB) was isolated using standard protocols. The construct was verified with restriction digests and sequence analysis. ‘p’ indicates the presence of the promoter region in front of angB to emphasize the presence of multiple promoter sites in the construct.

### Isolation of Plasmid pSG1448/27/91/4spnO5/2p4/193/6eryCIII (pSG144angAIangAIIangMIIIspnOorf14pangBangMIeryCIII)

Plasmid pSG1448/27/91/44/193/6eryCIII no9 was digested with BglII and the about 2 kb fragment was isolated and ligated with the BglII digested vector fragment of pSG1448/27/91/4spnO5/2p4/19 (pSG144angAIangAIIangMIIIspnOorf14pangB). The ligation was used to transform E. coli DH10B and plasmid pSG1448/27/91/4spnO5/2p4/193/6eryCIII (pSG144angAIangAIIangMIlIspnOorf14pangBangMIeryCIII) was isolated using standard protocols. The construct was verified with restriction digests and sequence analysis. EryCIII carries a his-tag fusion at the end. ‘p’ indicates the presence of the promoter region in front of angB to emphasize the presence of multiple promoter sites in the construct. The plasmid construct was used to transform mutant strains of S. erythraea using standard procedures.

### Bioconversion of 3-O-mycarosyl erythronolide B to 5-O-dedesosaininyl-5-O-angolosaininyl erythrornycins

Strain S. erythiaea Q42/1 pSG1448/27/91/4spnO5/2p4/193/6eryCIII was grown and bioconversions with fed 3-O-mycarosyl erythronolide B were performed as described in the General Methods. The cultures were analysed and peaks with m/z 704, m/z 718 and m/z 734 consistent with the presence of angolosaminyl erythromycin D, B and A, respectively, were observed.

### EXAMPLE 6

### Production of 5-O-angolosaminyl Yylactone

### Isolation of Plasmid pSG1448/27/91/AspnO5/2p4/193/6tylMII

(pSG144angAIangAIIangMIIIspnOorf14pangBangMItylMII)

Plasmid pSG1448/27/91/44/193/6tylMII no9 was digested with BglII and the about 2 kb fragment was isolated and ligated with the BglII digested vector fragment of pSG1448/27/91/4spnO5/2p4/19 (pSG144angaIangAIIangMIIIspnOorf14pangB). The ligation was used to transform E. coli DH10B and plasmid pSG 1448/27/91/4spnO5/2p193/6tylMII (pSG144angAIangAIIangMIIIspnOorf14pangBangMItylMIi) was isolated using standard protocols. The construct was verified with restriction digests and sequence analysis. TylMII carries a his-tag fusion at the end. The plasmid was used to transform mutant strains of S. erythraea applying standard protocols. ‘p’ indicates the presence of the promoter region in front of angB to emphasize the presence of multiple promoter sites in the construct.

### Isolation of S. erytlraea 18A1 (BIOT-2634)

To introduce a deletion comprising the PKS and majority of post PKS genes in S. erythraea a region of the left hand side of the ery- cluster (LHS) containing a portion of eryCl, the complete ermE gene and a fragment of the eryBI gene were cloned together with a region of the right hand side of the ery- cluster (RHS) containing a portion of the eryBVII gene, the complete eryK gene and a fragment of DNA adjacent to eryK. This construct should enable homologous recombination into the genome in both LHS and RHS regions resulting in the isolation of a strain containing a deletion between these two regions of DNA. The LHS fragment (2201 bp) was PCR amplified using S. erythraea chromosomal DNA as template and primers BldelNde (5′-CCCATATGACCGGAGTTCGAGGTACGCGGCTTG-3′, SEQ ID NO: 48) and BIdelSpe (5′-GATACTAGTCCGCCGACCGCACGTCGCTGAGCC-3′, SEQ ID NO: 49). Primer BIdeINde contains an NdeI restriction site (underlined) and primer BIdelSpe contains a SpeI restriction site used for subsequent cloning steps. The PCR product was cloned into the Smal restriction site of pUC19, and plasmid pLSB177 was isolated using standard procedures. The construct was confirmed by sequence analysis. Similarly, RHS (2158 bp) was amplified by PCR using S. erythraea chromosomal DNA as template and primers BVIIdelSpe (5′-TGCACTAGTGGCCGGGCGCTCGACGT CATCGTCGACAT-3′, SEQ ID NO: 50) and BVIIdelEco (5′-TCGATATCGTGTCCTGCGGTTTCACC TGCAACGCTG-3′, SEQ ID NO: 51). Primer BVIIdelSpe contains a SpeI restriction site and primer BVIIdelEco contains an EcoRV restriction site. The PCR product was cloned into the SinaI restriction site of pUC19 in the orientation with SpeI positioned adjacent to KpnI and EcoRV positioned adjacent to xbaI. The plasinid pLSB 178 was isolated and confirmed using sequence analysis. Plasmid pLSB177 was digested with NdeI and SpeI, the ˜2.2 kb fragment was isolated and similarly plasmid pLSB178 was digested with NdeI and SpeI and the 4.6 kb fragment was isolated using standard methods. Both fragments were ligated and plasmid pLSB188 containing LHS and RHS combined together at a SpeI site in pUC19 was isolated using standard protocols. An NdeI/XbaI fragment (˜4.4 kbp) from pLSB188 was isolated and ligated with SpeI and NdeI treated pCJR24. The ligation was used to transform E. coli DH10B and plasmid pLSB189 was isolated using standard methods. Plasmid pLSB189 was used to transform S. erythraea P2338 and transformants were selected using thiostrepton. S. erythraea Del18 was isolated and inoculated into 6 ml TSB medium and grown for 2 days. A 5% inoculum was used to subculture this strain 3 times. 100 μof the final culture were used to plate onto R2T20 agar followed by incubation at 30° C. to allow sporulation. Spores were harvested, filtered, diluted and plated onto R2T20 agar using standard procedures. Colonies were replica plated onto R2T20 plates with and without addition of thiostrepton. Colonies that could no longer grow on thiostrepton were selected and further grown in TSB medium. S. erythraea 18A1 was isolated and confirmed using PCR and Southern blot analysis. The strain was designated LB-1 /BIOT-2634. For further analysis, the production of erythromycin was assessed as described in General Methods and the lack of erythromycin production was confirmed. In bioconversion assays this strain did not further process fed erythronolide B and erythromycin D was hydroxylated at C12 to give erythromycin C as expected, indicating that EryK was still functional.

### Bioconversion of Tylactone to5-O-angolosaminyl Tylactone

Strain S. erythraea SGQ2pSG1448/27/91/4spnO5/2p4/193/6tylM-III was grown and bioconversions with fed tylactone were performed as described in the General Methods. The cultures were extracted and analysed. A compound consistent with the presence of angolosaminyl tylactone was detected. 20 mg of this compound were purified and the structure was confirmed by NMR analysis. A compound consistent with the presence of angolosaminyl tylactone was also obtained when the gene cassette pSG1448/27/91/4spnO5/2p4/193/6tylMII was expressed in the S. erythraea strain Q42/1 or S. erythraea 18A1.

TABLE III
NMR data for 5-O-βD angolosaminyl Tylactone

| # | δ_{c} | δ_{H} (mult., Hz) | COSY H-H | HMBC H-C |
|---|---|---|---|---|
| 1 | 174.4 | | | |
| 2 | 39.8 | 1.91 d (16.8) | 2b | 1, 3 |
| | | 2.46 dd(16.8, 10.5) | 2a, 3 | 1 |
| 3 | 66.9 | 3.68 dd (10.5, 1.2) | 2b | 1 |
| 4 | 40.4 | 1.56 m | 5, 18 | 3 |
| 5 | 80.7 | 3.76 d (10.3) | 4 | 4, 7, 18, 19, 1′ |
| 6 | 38.7 | 2.68 m | 7b | |
| 7 | 33.6 | 1.45 m | | |
| | | 1.55 m | 6 | |
| 8 | 45.0 | 2.70 m | 21 | |
| 9 | 203.9 | | | |
| 10 | 118.3 | 6.26 d (15.5) | 11 | 12 |
| 11 | 147.7 | 7.27 d (15.5) | 10 | 9, 12, 13, 22 |
| 12 | 133.5 | | | |
| 13 | 145.4 | 5.60 d (10.4) | 14, 22 | 11, 14, 22, 23 |
| 14 | 38.3 | 2.70 m | 13, 15, 23 | 12, 13, 15, 23 |
| 15 | 78.8 | 4.68 td (9.7, 2.4) | 14, 16b | 1, 17 |
| 16 | 24.7 | 1.55 m | 15, 16b, 17 | 15 |
| | | 1.82 ddd | 16a, 17 | 18 |
| 17 | 9.6 | 0.91 t (7.2) | 16 | 15, 16 |
| 18 | 9.7 | 0.91 d (7.2) | 4 | 3, 4, 5 |
| 19 | 21.0 | 1.55 m | 20 | |
| 20 | 11.8 | 0.83 t (7.2) | 19 | 6, 19 |
| 21 | 17.1 | 1.15 d (6.8) | 8 | 7, 9 |
| 22 | 13.0 | 1.76 s | 13 | 11, 12, 13 |
| 23 | 16.1 | 1.05 d (6.5) | 14 | 13, 14, 15 |
| 1′ | 101.0 | 4.41 d (8.6) | 2′ | 2′ |
| 2′ | 28.0 | 1.48 m | 1′, 2b′, 3′ | 1′, 3′, 4′ |
| | | 2.05 ddd (10.4, 3.9, 1.6) | 2a′, 3′ | 1′, 3′ |
| 3′ | 65.8 | 2.89 td (10.0, 3.9) | 2a′, 2b′, 4′ | 4′ |
| 4′ | 70.5 | 3.16 dd (9.5, 9.0) | 3′, 5′ | 3′, 5′, 6′ |
| 5′ | 73.2 | 3.26 dq (9.6, 6.0) | 4′, 6′ | |
| 6′ | 17.7 | 1.3 d (6.0) | 5′ | |

### Isolation of Plasmid pSG1448/27/91/4spnOp5/2 (pSG144angAIang/AIIangMIIIspnOpangorf14)

Plasmid pSGLit35/2 (isolated from E. coli ET12567) was digested with XbaI and the insert fragment was isolated and ligated with the XbaI digested vector fragment of pSG1448/27/91/4spnO (pSG144angAIangAIIangMIIspnO). The ligation was used to transform E. coli DH10B and plasmid pSG1448/27/91/4spnOp5/2 (pSG144angAIangAIangMIIIspnOpangorf14) was isolated using standard protocols. The construct was verified with restriction digests and sequence analysis.

### Isolation ofplasmidpSG1448/27/91/4spnOp5/24/19 (pSG144angAIangAIIangMIIIspnOpangorf14angB)

Plasmid pSGLit24/19 (isolated from E. coli ET12567) was digested withXbaI and the insert fragment was isolated and ligated with the XbaI digested vector fragment of pSG1448/27/91/4spnCp5/2 (pSG144angAIangAIIangMIIspnOpangorf14). The ligation was used to transform E. coli DH10B and plasmid pSG1448/27/91/4spnOp5/24/19 (pSG144angaIangAIIangMIIIspnOpangorf194angB) was isolated using standard protocols. The construct was verified with restriction digests and sequence analysis.

### Isolation of Plasmid pSG1448/27/91/4spnOp5/24/193/6

(pSG144angAIangAIIangMIIIspnOpangorf14angBangMI)

Plasmid pSGLit23/6 (isolated from E. coli ET12567) was digested with XbaI and the insert fragment was isolated and ligated with the xbaI digested vector fragment of pSG1448/27/91/4spnOp5/24/19 (pSG144angAIangAIIangMII-spnOpangorf14angB). The ligation was used to transform E. coil DH10B and plasmid pSG1448/27/91/4spnOp5/24/193/6 (pSG144angAIangAIfangMIIIspnOpangorf14angBangMI) was isolated using standard protocols. The construct was verified with restriction digests and sequence analysis.

### Isolation of Plasmid pSG1448/27/91/4spnOp5/24/193/6angMII

(pSG144angAIangAIIangMIIIspnOpangorf14angBangMIangMII)

Plasmid pSGLit1angMII (isolated from E. coli ET12567) was digested with XbaI/BglII and the insert fragment was isolated and ligated with the XbaI and partial BglII digested vector fragment of pSG1448/27/91/4spnOp5/24/193/6 (pSG144angAIangAIIangMIIIspnOpangorf14angBangMI). The ligation was used to transform E. coli DH10B and plasmid pSG1448/27/91/4spnOp5/24/193/6angMII (pSG144angAIangAIIangMIIIspnOpangorf14angBangMIangMII) was isolated using standard protocols. The construct was verified with restriction digests and sequence analysis. The plasmid was used to transform mutant strains of S. erytlraea with standard procedures.

### Biotransformation using S. erythraea Q42/1 pSG1448/27/91/4spnOp5/24/193/6angMII

(pSG144angAIangAIIangMIIIspnOpangorf14angBangMIangMII)

Biotransformation experiments feeding tylactone are carried out as described in General Methods and the cultures are analysed. Angolosaminyl tylactone is detected.

### Isolation of Plasmid pSG1448/27/96/4 (pSG144angAIangAIIangorf4)

Plasmid pSG1448/27/9 (pSG144angAIangA14) was digested with XbaI and treated with alkaline phosphatase using standard protocols. The vector fragment was used for ligations with XbaI treated plasmid pSGLit26/4 no9 followed by transformations of E. coli DH10B using standard protocols. Plasmid pSGI448/27/96/4 (pSG144angalangAIIangorf4) was isolated using standard procedures and the construct was confirmed by restriction digests and sequence analysis.

### Isolation of Plasmid pSG1448/27/96/4p5/2 (pSG144angAIangAIIangorf4pangorf14)

Plasmid pSGLit35/2 (isolated from E. coli ET12567) was digested with XbaI and the insert fragment was isolated and ligated with the XbaI digested vector fragment of pSGI448/27/96/4 (pSG144angAIangAIIangorf4). The ligation was used to transform E. coli DH10B and plasmid pSG1448/27/96/4p5/2 (pSG144angAIangAIIangorf4pangorf14) was isolated using standard protocols. The construct was verified with restriction digests and sequence analysis.

### Isolation of Plasmid pSG1448/27/96/4p5/21/4 (pSG144ang/AIangAIIangorf4pangorf14angMIII)

Plasmid pSGLit21/4 (isolated from E. coli ET12567) was digested with XbaI and the 1.4 kb fragment was isolated and ligated with the XbaI digested vector fragment of pSG1448/27/96/4p5/2 (pSG144angaIangAIIangorf4pangorf14). The ligation was used to transform E. coli DH10B and plasmid pSG1448/27/96/4p5/21/4 (pSG144angAIangAIIangorf4pangorf14angMIII) was isolated using standard protocols. The construct was verified with restriction digests and sequence analysis.

### Isolation of Plasmid pSG1448/27/96/4p5/21/44/19 (pSG144angAIangAIIangorf4pangorf14angMIIIangB)

Plasmid pSGLit24/19 (isolated from E. coli ET12567) was digested with XbaI and the 1.4 kb fragment was isolated and ligated with the XbaI digested vector fragment of pSG1448/27/96/4p5/21/4 (pSG144angAIangAIIangorf4pangorf14angMIII). The ligation was used to transform E. coli DH10B and plasmid pSG1448/27/96/4p5/21/44/19 (pSG144angAIangAIIangorf4pangorf14angMIIIangB) was isolated using standard protocols. The construct was verified with restriction digests and sequence analysis.

### Isolation of Plasmid pSG1448/27/96/4p5/21/44/193/6angMII

(pSG144a ngAIangAIIangorf4pangorf14angMIIIangBangMIangMII)

Plasmid pSG1448/27/91/4spnOp5/24/193/6angMI was digested with BglII and the about 2.2 kb fragment was isolated and used to ligate with the BglII treated vector fragment of SG1448/27/96/4p5/21/44/19. The ligation was used to transform E. coli DH10B using standard procedures and plasmid pSG1448/27/96/4p5/21/44/193/6angMII (pSG144angAIangAIIangorf4pangorf14angMIIIangBangMIangMII) was isolated. The construct was verified using restriction digests and sequence analysis. The plasmid was used to transform mutant strains of S. erythraea with standard protocols.

### Bioconversion of Tylactone with S. erythraea Q42/1 pSG1448/27/96/4p5/21/44/193/6angMII

(pSG144angAIangAIIangorf4pangorf14angMIIIangBangMIangMII)

Biotransformation experiments feeding tylactone are carried out as described in General Methods and the cultures are analysed. Angolosaminyl tylactone is detected.

### EXAMPLE 7

### Cloning of eryK into the Gene Cassette pSG144

### Isolation of Plasmid pUC19eryK

To amplify eryK primers eryK1 5′-GGTCTAGACTACGCCGACTGCCTCGGCGAGGAGCCC-3′ (SEQ ID NO: 52) and eryK2: 5′-GGCATATGTTCGCCGACGTGGAAACGACCTGCTGCG-3′ (SEQ ID NO: 53) were used and the PCR product was cloned as described for pUC19eryCVI. Plasmid pUC19eryK was isolated.

### Isolation of Plasmid pLSB111 (pCJR24eryK)

Plasmid pUC19eryK was digested with NdeI/XbaI and the insert band was ligated with NdeI/XbaI digested pCJR24. Plasmid pLSB111 (pCJR24eryK) was isolated and the construct was verified with restriction digests.

### Isolation of Plasmid pLSB115

Plasmid pLSB111 (pCJR24eryK) was digested with NdeI/XbaI and the insert fragment was isolated and ligated with the NdeI/XbaI digested vector fragment of plasmid pSGLit2 and plasmid pLSB115 was isolated using standard protocols. The plasmid was verified using restriction digestion and DNA sequence analysis.

### Isolation of Plasmid pSG1448/27/95/21/4eryK

Plasmid pLSB115 from E. coli ET12567 was digested with XbaI and the insert fragment was isolated and ligated with the XbaI treated vector fragment of pSG1448/27/95/21/4 (pSG144angAIangAIIangorf14angMIII). The ligation was used to transform E. coli DH10B with standard procedures and plasmid pSG1448/27/95/21/4eryK (pSG144angAIangAIangorf14angMIIIeryK) is isolated. The construct is confirmed with restriction digests.

### Isolation of plasmid pSG1448/27/95/21/4eryK4/19

Plasmid pSGLit24/19 from E. coli ET12567 is digested with XbaI and the insert fragment is isolated and ligated with the xbaI treated vector fragment of plasmid pSG1448/27/95/21/4eryK. The ligation is used to transform E. coli DH10B with standard procedures and plasmid pSG1448/27/95/21/4eryK4/19 (pSG144angAIangtlIIangorf14angMIIIeryKangB) is isolated. The construct is confirmed with restriction digests.

### Isolation of PlasmidpSG1448/27/95/21/4eryK4/193/6eryCIII

Plasmid pSG1448/27/95/21/44/193/6eryCIII is digested with BglII and the about 2.1 kb fragment is isolated and ligated with the BglII treated vector fragment of pSG1448/27/95/21/4eryK4/19. Plasmid pSG1448/27/95/21/4eryK4/193/6eryCIII is isolated using standard procedures and the construct is confirined using restriction digests. The plasmid is used to transform mutant strains of S. erythraea with standard methods.

### Bioconversion of 3-O-mycarosyl eiythronolide B to 5-O-dedesosaminyl-5-O-mycamninosyl erythromycin A

The S. erythraea strain Q4211pSG1448/27/95/21/4eryK4/193/6eryCIII is grown and bioconversions with fed 3-O-mycarosyl erythronolide B are performed as described in the General Methods. The cultures are analysed and a compound with m/z 750 is detected consistent with the presence of 5-O-dedesosaminyl-5-O-mycaminosyl erythromycin A.

### EXAMPLE 8

### Production of 13-desethyl-13-methyl-5-O-mycaminosyl erythromycins A and B; 13-desethyl-13-isopropyl-5-O-mycaminosyl erytliromycin A and B; 13-desethyl-13-secbutyl-5-O- mycaminosyl erythromycin A and B

### Production of 13-desethyl-13-methyl-3-O-nziycarosyl erythronolide B, 13-desethyl-13-isopropyl-3-O-mycarosyl erythronolide B and 13-desethyl-13-secbutyl-3-O-mycarosyl erytlronotide B

Plasmid pLS025, (WO 03/033699) a pCJR24-based plasmid containing the DEBS1, DEBS2 and DEBS3 genes, in which the loading module of DEBS1 has been replaced by the loading module of the avermectin biosynthetic cluster, was used to transform S. erythraea JC2ΔeryCIII (isolated using techniques and plasmids described previously (Rowe et al., 1998; Gaisser et al., 2000)) using standard techniques. The transformant JC2ΔeryCIIIpLS025 was isolated and cultures were grown using standard protocols. Cultures of S. erythraea JC2ΔeryCIIIpLS025 are extracted using methods described in the General Methods section and the presence of 3-O-mycarosyl erythronolide B, 13-desethyl-13-methyl-3-O-mycarosyl erythronolide B, 13-desethyl-13-isopropyl-3-O-mycarosyl erythronolide B and 13-desethyl-13-secbutyl-3-O-mycarosyl erythronolide B in the crude extract is verified by LCMS analysis.

Production of 13-desetdyl-13-methyl-5-O-dedesosminyl-5-O-rnycaminosyl erythromycin A and B, 13- desetlyl-13-isopropyl-5-O-dedesosaininyl-5-O-mycaininosyl erythromnycin A and B, 13-desethyl-13- secbutyl-5-O-dedesosminyl-5-O-mycaminosyl erythromycin A and B

Cultures of S. erythraea JC2ΔeryCIIIpLS025 are extracted using methods described in the General Methods section and the crude extracts are dissolved in 5 ml of methanol and subsequently fed to culture supernatants of the S. erythraea strain SGQ2pSG1448/27/95/21/44/193/6eryCIII using standard techniques. The bioconversion of 13-desethyl-13-methyl-3-O-mycarosyl erythronolide B, 13-desethyl-13- isopropyl-3-O-mycarosyl erythronolide B and13-desethyl-13-secbutyl-3-O-mycarosyl erythronolide B to 13-desethyl-13-metlyl-5-O-dedesosaminyl-5-O-mycaminosyl erythromycin A and 13-desethyl-13-mnethyl-5-O-dedesosaminyl-5-O-mycaminosyl erythromycin B; 13-desethyl-13-isopropyl-5-O-dedesosaminyl-5-O-mycaminosyl erythromycin A and 13-desethyl-13-isopropyl-5-O-dedesosaminyl-5-O-mycaminosyl erythromycin B; 13-desethyl-13-secbutyl-5-O-dedesosaminyl-5-O-mycaminosyl erythrornycini A and 13-desethyl-13-secbutyl-5-O-dedesosaminyl-5-O-mycaminosyl erythromycin B is verified by LCMS analysis.

### EXAMPLE 9

### 13-desethyl-13-methyl-5-O-dedesosaminyl-5-O-mycaminosyl erythromycin A and 13-desethyl-13-methyl-5-O-dedesosaminyl-5-O-mycaminosyl erythromycin B

### Production of 13-desethyl-13-inethyl-3-O-inycarosyl erythronolide B

Plasmid pIB023 (Patent application no 0125043.0), a pCJR2-based plasmid containing the DEBS1, DEBS2 and DEBS3, was used to transform S. erythraea JC2ΔeryCIII using standard techniques. The transformant JC2ΔeryCIIIpIB023 was isolated and cultures were grown using standard protocols, extracted and the crude extract was assayed using methods described in the General Methods section. The production of 3-O-mycarosyl erythronolide B, and 13-desethyl-13-methyl-3-O-mycarosyl erythronolide B is verified by LCMS analysis.

### Production of 13-desethyl-13-inethyl-5-O-dedesosaininyl-5-O-inycarninosyl erythromycin A, 13-desethyl-13-methyl-5-O-dedesosaminyl-5-O-mycaminosyl erythromycin B

Cultures of S. erythraea JC2ΔeryCIIIpIB023 are extracted using methods described in the General Methods section and the crude extracts are dissolved in 5 ml of methanol and subsequently fed to culture supernatants of S. erythraea SGQ2pSG1448/27/95/21/44/193/6eryCIII using standard techniques. The bioconversion of 13-desethyl-13-methyl-3-O-mycarosyl erythronolide B to 13-desethyl-13-methyl-5-O-dedesosaminyl-5-O-mycaminosyl erythromycin A and 13-desethyl-13-methyl-5-O-dedesosaminyl-5- O-mycaminosyl erythromycin B are verified by LCMS analysis.

### EXAMPLE 10

### Production of 5-O-dedesosaminyl-5-O-mycaminosyl azithromycin

Azithromycin aglycones were prepared using methods described in EP1024145A2 (Pfizer Products Inc. Groton, Connecticut). The S. erythraea strain SGT2pSG142 was isolated using techniques and plasmid constructs described earlier (Gaisser et al., 2000). Feeding experiments are carried out using methods described previously (Gaisser et al., 2000) with the S. erythraea mutant SGT2pSG142 thus converting azithromyciin aglycone to 3-O-mycarosyl azithronolide. Biotransformation experiments are carried out using S. erythraea SGQ2pSG1448/27/95/21/44/193/6eryCIII and crude extracts containing 3-O-mycarosyl azithronolide are added using standard microbiological techniques. The bioconversion of 3-O-mycarosyl azithronolide to 5-O-dedesosaminyl-5-O-mycaminosyl azithromycin is verified by LCMS analysis.

### EXAMPLE 11

### Production of 5-O-dedesosaminyl-5-O-mycaminosyl erythromycin C

### Isolation of the S. erythraea mutant SGP1 (SGQ2←eryG)

To create a chromosomal deletion in eryG, construct pSGAG3 was isolated as follows:

Fragment 1 was amplified using primers BIOSG53 5′-GGAATTCGGCCAGGACGCGTGGCTGGTCACCGGCT-3′ (SEQ ID NO: 54) and BIOSG54 5′-GGTCTAGAAAGAGCGTGAGCAGGCTCTTCTACAGCCAGGTCA-3′ (SEQ ID NO: 55) and genomic DNA of S. erythraea was used as template. Fragment 2 was amplified using primers

BIOSG55

(SEQ ID NO: 56)

5′-GGCATGCAGGAAGGAGAGAACCACGATGACCACCGACG-3′
and
BIOSG56

(SEQ ID NO: 57)

5′-GGTCTAGACACCAGCCGTATCCTTTCTCGGTTCCTCTTGTG-3′

and genomic DNA of S. erythaea was used as template. Both DNA fragments were cloned into SinaI cut pUC19 using standard techniques, plasmids pUCPCR1 and pUCPCR2 were isolated and the sequence of the amplified fragments was verified. Plasmid pUCPCR1 was digested using EcoRI/XbaI and the insert band DNA was isolated and cloned into EcoRI/XbaI digested pUC19. Plasmid pSGAG1 is isolated using standard methods and digested with SphI/XbaI followed by a ligation with the SphI/XbaI digested insert fragment of pUCPCR2. Plasmid pSGAG2 is isolated using standard procedures, digested with SphI/HindIII and ligated with the SphI/HindIII fragment of pCJR24 (Rowe et al., 1998) containing the gene encoding for tlhiostrepton resistance. Plasmid pSGAG3 is isolated and used to delete eryG in the genome of S. erythraea strain SGQ2 using methods described previously (Gaisser et al., 1997; Gaisser et al., 1998) and the S. erythraea mutant SGP1 (SGQ2ΔeryG) is created.

Production of 5-O-dedesosaminyl-5-O-mycamninosyl erythromycin C

The S. erythraea strain SGP1 (S. eiythraea SGQ2ΔeryG) is isolated using standard techniques and consequently used to transform the cassette construct pSG1448/27/95/21/44/193/6eryCIII as formerly described. The S. erythraea strain SGPlpSG1448/27/95/21/44/193/6eryCIII is isolated and used for biotransformation as described in Example 2 and assays are carried out as described above to verify the conversion of 3-O-mycarosyl-erythronolide B to 5-O-dedesosaminyl-5-O-mycaminosyl erythromycin C by LCMS analysis.

### EXAMPLE 12

### Production of 3-O-angolosaminyl-erythronolide B

### Bioconversion of Erythronolide B with S. erythaea Q42/1 pSG1448/27/91/4spnOp5/24/193/6angMII

(pSG144angAIangAIIangMIIIspnOpangorf14andBangMIangMII)

Biotransformation experiments feeding erythronolide B were carried out as described in General Methods and the cultures were analysed. Angolosaminylated erythronolide B was detected. About 30 mg of 3-O-angolosaminyl-erythronolide B were isolated and the structure was confirmed by NMR analysis.

TABLE IV
¹H and ¹³C NMR for the 3-angolosaminyl-erythronolide B in CDCl₃

| | | | | | H—C |
|---|---|---|---|---|---|
| Position | | δ_{C} | δ_{H} (mult., Hz) | H—H COSY | HMBC |
| 1 | COO | 176.3 | — | — | — |
| 2 | CH | 44.5 | 2.81 dq (10.4, 6.7) | 3, 16 | 1, |
| 3 | CH | 89.7 | 3.66 dd (10.5, 10.5) | 2, | 1, 2, 4, 5, |
| | | | | | 16, 17, 1′ |
| 4 | CH | 36.5 | 1.99 m | 17 | 5, 6, 17 |
| 5 | CH | 81.5 | 3.69 bs | | 3, 6, 7, 17, |
| | | | | | 18 |
| 6 | C | 75.2 | — | | — |
| 7 | CH₂ | 38.3 | 1.92 dd (14.6, 9.0) | 7b, 8 | 6, 8, 9, 18, |
| | | | | | 19 |
| | | | 1.44 dd (14.6. 5.4) | 7a, 8 | 6, 8, 9, 18 |
| 8 | CH | 43.4 | 2.69 m | 7 | 7, 9, 18 |
| 9 | CO | 217.8 | — | | — |
| 10 | CH | 40.1 | 2.91 bq (6.6) | 20 | 9, 11, 20 |
| 11 | CH | 70.6 | 3.78 d (10.0) | 12 | 12, 13, 20 |
| 12 | CH | 40.2 | 1.69 m | 11, 21 | 13, 21 |
| 13 | CH | 75.6 | 5.40 dd (9.5, 9.3) | 14 | 1, 11, 12, |
| | | | | | 14, 15, 21 |
| 14 | CH₂ | 25.8 | 1.71 qd (7.2, 2.2) | 13, 14b, 15 | 12, 13 |
| | | | 1.51 m | 13, 14a, 15 | 13 |
| 15 | CH₃ | 9.1 | 0.90 d (7.7) | 14 | |
| 16 | CH₃ | 15.2 | 1.19 d (6.9) | 2 | 2, 3 |
| 17 | CH₃ | 8.3 | 1.06 d (6.7) | 4 | 3, 4, 5 |
| 18 | CH₃ | 26.6 | 1.30 s | | 5, 6, 7 |
| 19 | CH₃ | 16.9 | 1.16 d (6.1) | | 1 |
| 20 | CH₃ | 8.5 | 0.98 t (7.7) | 10 | 9, 10, 11 |
| 21 | CH₃ | 10.4 | 0.89 d (7.7) | 12 | 11, 12, 13 |
| 1′ | CH | 103.0 | 4.61 dd (9.2, 1.6) | 2′ | 2′, 3′, 3 |
| 2′ | CH₂ | 27.0 | 1.49 m | 1′, 2b, 3′ | 1′, 3′ |
| | | | 2.00 m | 2a, 3′ | 1′, 3′, 4′ |
| 3′ | CH | 65.2 | 2.48 td (10.2, 3.5) | 2′, 4′ | 4′ |
| 4′ | CH | 70.3 | 3.03 dd (9.5, 9.5) | 3′, 5′ | 3′, 5′, 6′ |
| 5′ | CH | 73.9 | 3.34 dq (8.7, 6.0) | 4′, 6′ | 3′ |
| 6′ | CH₃ | 17.5 | 1.34 d (6.0) | 5′ | 4′, 5′ |

### Bioconversion of erythronolie B erythronolide B with S. erythraea 18A1 pSG1448/27/96/4p5/21/44/193/6angMII

(pSG144angAIangAIIangorf4pangorf14angMIIIangBangMIangMII)

Biotransformation experiments feeding erythronolide B were carried out as described in General Methods and the cultures are analysed. Peaks characteristic for angolosaminylated erythronolide B were detected.

### REFERENCES

Ali, N., Herron, P. R., Evans, M. C. and Dyson, P. J. (2002) Osmotic regulation of the Streptomyces lividans thiostrepton-inducible promoter, ptipA. Microbiology 148: 381-390.

Bertram, G., Innes, S., Minella, O., Richardson, J. P. and Stanfield, I. (2001) Endless possibilities: translation termination and stop codon recognition. Microbiology 147: 255-269.

Bussiere, D. E. and Bastia, D. (1999) Termination of DNA replication of bacterial and plasmid chromosomes. Mol Microbiol 31: 1611-1618.

Bierman, M., Logan, R., O'Brien, K., Seno, E. T., Rao, R. N. and Schoner, B. E. (1992) Plasmid cloning vectors for the conjugal transfer of DNA from Escherichia coli to Streptomyces spp. Gene 116: 43-49.

Caffrey, P., Bevitt, D. J., Staunton, J. and Leadlay, P. F. (1992) Identification of DEBS1, DEBS 2 and DEBS 3, the multienzyme polypeptides of the erythromycin-producing polyketide synthase from Saccharopolyspora erythraea. FEBS 304: 225-228.

Doumith, M., Legrand, R., Lang, C., Salas, J. and Raynal, M. C. (1999) Interspecies complementation in Saccharopolyspora erythraea: Elucidation of the function of oleP1, oleG1 and oleG2 from the oleandomycin biosynthetic gene cluster of Streptomyces antibioticus and generation of new erythromnycin derivatives. Mol Microbiol 34:1039-1048.

Gaisser, S., Böhm, G. A., Cortés, J. and Leadlay, P. F. (1997) Analysis of seven genes from the eryAI-eryK region of the erythromycin biosynthetic gene cluster in Saccharopolyspora erythraea. Mol Gen Genet 256: 239-251.

Gaisser, S., Böhm, G. A., Doumith, M., Raynal, M. C., Dhillon, N., Cortés, J. and Leadlay, P. F. (1998) Analysis of eryBI, eryBIII and eryBVII from the erythromycin biosynthetic gene cluster in Saccharopolyspora erythraea. Mol Gen Genet 258: 78-88.

Gaisser, S., Reather, J., Wirtz, G., Kellenberger, L., Staunton, J. and Leadlay, P. F. (2000) A defined system for hybrid macrolide biosynthesis in Saccharopolyspora erythraea. Mol Microbiol 36: 391-401.

Gaisser, S., Martin, C. J., Wilkinson, B., Sheridan, R. M., Lill, R. E., Weston, A. J., Ready, S. J., Waldron, C., Crouse, G. D., Leadlay, P. F. and Staunton, J. (2002a) Engineered biosynthesis of novel spinosyns bearing altered deoxyhexose substituents. Chemn Commun 618-619.

Gaisser, S., Lill, R., Staunton, J., Mendez, C., Salas, J. and Leadlay, P F. (2002b) Parallel pathways for oxidation of 14-membered polyketide macrolactones in Saccharopolyspora erythraea. Mol Microbiol 44: 771-81.

Gates, P. J., Kearney, G. C., Jones, R., Leadlay, P. F. and Staunton, J. (1999) Structural elucidation studies of erythromycins by electrospray tandem mass spectrometry. Rapid Commun Mass Spectrom 13: 242-246.

Hansen, J. L., Ippolito, J. A., Ban, N., Nissen, P., Moore, P. B. and Steitz, T. A. (2002) The structures of four macrolide antibiotics bound to the large ribosomal subunit. Molecular Cell 10: 117-128.

Hu Y. and Walker S. (2002) Remarkable structural similarities between diverse glycosyltransferases. Chemistry and Biology 9: 1287-1296.

Ichinose, K., Ozawa M., Itou K., Kunieda K., and Ebizuka Y. (2003) Cloning, sequencing and heterologous expression of the medermycin biosynthetic gene cluster of Streptomyces sp AM-7161: towards comparative analysis of the benzoisochromanequinone gene cluster. Microbiology 149: 1633-1645.

Jones, P. H., Iyer, K. S. and Grundy, W. E. (1969) Chemical modifications of erythromycin antibiotics. II. Synthesis of 4′-hydroxyerythromycin A. Antimicrobial Agents Chemother 9: 123-130.

Kaneko, T., McArthur, H. and Sutcliffe, J. (2000) Recent developments in the area of macrolide antibiotics. Exp Opin Ther Patents 10: 1-23.

Kato, Y., Bai, L., Xue, Q., Revill, W. P., Yu, T. W. and Floss, H. G. (2002) Functional expression of genes involved in the biosynthesis of the novel polyketide chain extension unit, methoxymalonyl-acyl carrier protein, and engineered biosynthesis of 2-desmethyl-2-methoxy-6-deoxyerythronolide B. J. Am Chem Soc 124: 5268-5269.

Kieser, T., Bibb, M. J., Buttner, M. J., Chater, K. F., and Hopwood, D. A. (2000) Practical Streptomyces Genetics, John Innes Foundation, Norwich.

Kinumaki, A. and Suzuki, M. (1972) Proposed structure of angolamycin (shincomycin A) by mass spectrometry. J. Antibiotics 25: 480-482.

Lee, M. H., Pascopella, L., Jacobs, W. R. Jr, and Hatfull, G. F. (1991). Site specific integration of mycobacteriopliage L5: integration-proficient vectors for Mycobacterium smegmatis, Mycobacterium tuberculosis and Bacille Calmette-Guerin. Proc. Natl. Acad. Sci. USA, 88: 3111-3115.

Liu, H.-W. and Thorson, J. S. (1994) Pathways and mechanisms in the biogenesis of novel deoxysugars by bacteria. Annu Rev Microbiol 48: 223-56.

Madduri, K., Kennedi, J., Rivola, G., Inventi-Solari, A., Filippini, S., Zanoso, G., et al., (1998) Production of the antitumor drug epirubicin (4′-epidoxorubicin) and its precursor by a genetically engineered strain of Streptomyces peucetius. Nat Biotechnol 16: 69-74.

Matsuura, M., Noguchi, T., Yamaguchi, D., Aida, T., Asayama, M., Takahashi, H. and Shirai, M. (1996). The sre gene (ORF469) encodes a site-specific recombinase responsible for integration of the R4 phage genome. J Bact. 178: 3374-3376.

Méndez, C. and Salas, J. A. (2001) Altering the glycosylation pattern of bioactive compounds. Trends in Biotechnology 19: 449-456.

Pfoestl, A., Hofinger, A., Kosma, P., and Messner P. (2003) Biosynthesis of dTDP-3-acetamido-3,6-dideoxy-alpha-D-galactose in Aneurinibacillus thernioaerophilus L420-91^{T}*. J Bio Chem 278:26410-26417.

Poulsen, S. M., Kofoed, C. and Vester, B. (2000) Inhibition of the ribosomal peptidyl transferease reaction by the mycarose moiety of the antibiotics carbomycin, spiramycin and tylosin. J Mol Biol 304: 471-481.

Rowe, C. J., Cortés, J., Gaisser, S., Staunton, J. and Leadlay, P. F. (1998) Construction of new vectors for high-level expression in actinomycetes. Gene 216: 215-223.

Salah-Bey, K., Blanc, V. and Thompson, C. J. (1995) Stress-activated expression of a Streptomyces pristinaespiralis multidrug resistance gene (ptr) in various Streptomyces spp. and Escherichia coli. Molecular Microbiology 17: 1001-1012.

Salah-Bey, K., Doumith, M., Michel, J. M., Haydock, S., Cortés, J., Leadlay, P. F. and Raynal, M. C. (1998) Targeted gene inactivation for the elucidation of the deoxysugar biosynthesis in the erythromycin producer Saccharopolyspora erythraea. Mol Gen Genet 257: 542-553.

Sambrook, J., Fritsch, E. F. and Maniatis, T. (1989) Molecular cloning: a laboratory manual, 2nd ed. Cold Spring Harbor Laboratory Press, N.Y.

Schliünzen, F., Zarivach, R., Harms, J., Bashan, A., Tocilj, A., Albrecht, R., Yonath, A. and Franceschi, F. (2001) Structural basis for the interaction of antibiotics with the peptidyl transferase centre in eubacteria. Nature 413: 814-821.

Solenberg, P. J., Matsushima, P., Stack, D. R., Wilkie, S. C., Thompson, R. C. and Baltz, R. H. (1997) Production of hybrid glycopeptide antibiotics in vitro and in Streptomnyces toyocaensis. Chem Biol 4: 195-202.

Smovkina, T., Mazodier, P., Boccard, F., Thompson, C. J. and Guerineau, M. (1990) Construction of a series of pSAM2-based integrative vectors for use in actinomycetes. Gene 94: 53-59.

Spagnoli, R., Cappelletti, L. and Toscano, L. (1983) Biological conversion of erythronolide B, an intermediate of erythromycin biogenesis, into “hybrid” macrolide antibiotics. J Antibiot 36: 365-375.

Staunton, J. and B. Wilkinson (1997). Biosynthesis of erythromycin and rapamycin. Chem Rev 97: 2611-2629.

Summers, R. G., Donadio, S., Staver, M. J., Wendt-Pienkowski, E., Hutchinson, C. R. and Katz, L. (1997) Sequencing and mutagenesis of genes from the erythromycin biosynthetic gene cluster of Saccharopolyspora erythraea that are involved in L-mycarose and D-desosamine production. Microbiol 143: 3251-3262.

Tang, L. and McDaniel, R. (2001) Construction of desosamine containing polyketide libraries using a glycosyltransferase with broad substrate specificity. Chemistry and Biology 8: 547-555.

Trefzer, A., Salas, J. A. and Bechthold, A. (1999) Genes and enzymes involved in deoxysugar biosynthesis in bacteria. Nat Prod Rep 16: 283-299.

Van Mellaert, L., Mei, L., Lammertyn, E., Schacht, S., and Anne, J. (1998) Site-specific integration of bacteriophage VWB genome into Streptomyces venezuelae and construction of a VWB-based integrative vector. Microbiology 144: 3351-3358.

Wohlert, S. E., Blanco, G., Lombo, F., Fernandez, E., Brana, A. F., Reich, S., Udvarnoki, G., et al., (1998) Novel hybrid tetracenomycins through combinatorial biosynthesis using a glycosyltransferase encoded by the elm genes in cosmid 16F4 and which shows a broad sugar substrate specificity. J Am Chem Soc 120: 10596-10601.

Wohlert, S.E., Lomovskaya, N., Kulowski, K., Fonstein, L., Occi, J. L., Gewain, K. M., MacNeil, D. J. and Hutchinson, C. R. (2001) Insights about the biosynthesis of the avermectin deoxysugar L-oleandrose through heterologous expression of Streptomyces avermitilis deoxysugar genes in Streptomyces lividans. Chemistry & Biology 8: 681-700.

Zhao, L., Ahlert, J., Xue, Y., Thorson J.S., Sherman, D. H. and Liu, H-W. (1999) Engineering a methymycin/pikroinycin-calicheamicin hybrid: Construction of two new macrolides carrying a designed sugar moiety. J Am Chem Soc 121: 9881-9882.

Zhao, L., Que, N. L. S, Xue, Y., Sherman, D. H. and Liu, H. W. (1998a) Mechanistic studies of desosamine biosynthesis: C-4 deoxygenation precedes C-3 transamination. J Am Chem Soc 120: 12159-10160.

Zhao, L., Sherman, D. H. and Liu, H. W. (1998b) Biosynthesis of desosamine: Construction of a new methymycin/neomethymycin analogue by deletion of a desosamine biosynthetic gene. J Am Chem Soc 120: 10256-10257.

1. A gene cassette comprising a combination of genes which, in an appropriate strain background, are able to direct the synthesis of mycaminose or angolosamine and to direct its subsequent transfer to an aglycone or pseudoaglycone.

2. A gene cassette according to claim 1, comprising a combination of genes able to direct the synthesis and transfer of mycaminose, wherein:
a) at least one of the genes is selected from the group consisting of: angorfl4, tylmIl, tylMI, tylB, tylAl, tylAll, tylIa, angAI, angAII, angMIII, angB, angMI, eryG and eryK; and,
b) at least one of the genes is a glycosyltransferase gene selected from the group consisting of tylMII, angMII, desVII, eryC-II, eryBV, spnP, and midI.

3. A gene cassette according to claim 2, wherein one of the genes within the gene cassette is tylIa

4. A gene cassette according to claim 2, wherein one of the genes within the gene cassette is angorf14

5. A gene cassette according to claim 2, which comprises angAI, angAII, angorf14, angMIII, angB and angMI, in combination with one or more glycosyltransferase genes selected from the group consisting of eryCIII, tylMII and angMII.

6. A gene cassette according to claim 2, which comprises tylAI, tylAII, tylMIII, tylB, tylIa and tylMI, in combination with one or more glycosyltransferase genes selected from the group consisting of eryCIII, tylMII and angMII.

7. A gene cassette according to claim 1 comprising a combination of genes able to direct the synthesis and transfer of angolosamine, wherein:
a) at least one of the genes is selected from the group consisting of: angMIII, angMI, angB, angAI, angAII, angorf14, angorf4, tylMIII, tylMI, tylB, tylAI, tylAII, erytCVI, spnO, eryBVI, and eryK; and,
b) at least one of the genes is a glycosyltransferase gene selected from the group consisting of eryCIII, tylMII, angMII, desVII, eryBV, spnP and midI.

8. A gene cassette according to claim 7, which comprises angMIII, angMI, angB, angAl, angAIl, angorf14 and spnO, in combination with one or more glycosyltransferase genes selected from the group consisting of angMII, tylMII and eryCIII.

9. A gene cassette according to claim 7, which comprises angMIII, angMI, angB, angAI, angAII, angorf4, and angorfl4, in combination with one or more glycosyltransferase genes selected from the group consisting of angMII, tylMlI and eryCIII.

10. A process for the production of erythromycins and azithromycins which contain either mycaminose or angolosamine at the C-5 position, said process comprising transforming a strain with a gene cassette of claim 1 and culturing the strain under appropriate conditions for the production of said erythromycin or azithromycin.

11. The process of claim 10, wherein the strain is selected from actinomycetes, Pseudomonas, myxobacteria, and E. coli.

12. The process of claim 10, wherein the host strain is additionally transformed with the ermE from S. erythraea.

13. The process of claim 10, wherein the host strain is an actinomycete.

14. The process of claim 13, wherein the host strain is selected from S. erythraea, Streptomyces griseofuscus, Streptomyces cinnamonensis, Streptomyces albus, Streptomyces lividans, Streptomyces hygroscopicus sp., Streptomyces hygroscopicus var. ascomyceticus, Streptomyces longisporoflavus, Saccharopolyspora spinosa, Streptomyces tsukubaensis, Streptomyces coelicolor, Streptomyces fradiae, Streptomyces rimosus, Streptomyces avermitilis, Streptomyces eurythermus, Streptomyces venezuelae, and Amycolatopsis mediterranei.

15. The process of claim 14, wherein the host strain is S. erythraea.

16. The process of claim 15, wherein the host strain is selected from the SGQ2, Q42/1 or 18A1 strains of S. erythraea.

17. The process of claim 10, which further comprises feeding of an aglycone and/or a pseudoaglycone substrate to the recombinant strain.

18. The process of claim 17, wherein said aglycone and/or pseudoaglycone is selected from the group consisting of 3-O-mycarosyl erythronolide B, erythronolide B, 6-deoxy erythronolide B, 3-O-mycarosyl-6-deoxy erythronolide B, tylactone, spinosyn pseudoaglycone, 3-O-rhamnosyl erythronolide B, 3-O-rhamnosyl-6-deoxy erythronolide B, 3-O-angolosaminyl erythronolide B, 15-hydroxy-3-O-mycarosyl erythronolide B, 15-hydroxy erythronolide B, 15-hydroxy-6-deoxy erythronolide B, 15-hydroxy-3-O-mycarosyl-6-deoxy erythronolide B, 15-hydroxy-3-O-rhamnosyl erythronolide B, 15-hydroxy-3-O-rhamnosyl-6-deoxy erythronolide B, 15-hydroxy-3-O-angolosaminyl erythronolide B, 14-hydroxy-3-O-mycarosyl erythronolide B, 14-hydroxy erythronolide B, 14-hydroxy-6-deoxy erythronolide B, 14-hydroxy-3-O-mycarosyl-6-deoxy erythronolide B, 14-hydroxy-3-O-rhamnosyl erythronolide B, 14-hydroxy-3-O-rhamnosyl-6-deoxy erythronolide B, 14-hydroxy-3-O-angolosaminyl erythronolide B.

19. The process of claim 10, which additionally comprises the step of isolating the compound produced.

20. A compound according to the formula I below: 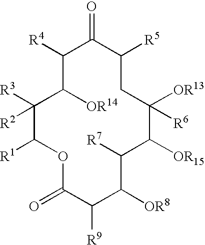
R¹ is selected from:
H, CH₃, C₂H₅
an alpha-branched C₃-C₈ group selected from alkyl, alkenyl, alkynyl, alkoxyalkyl and alkylthioalkyl groups any of which may be optionally substituted by one or more hydroxyl groups;
a C₅-C₈ cycloalkylalkyl group wherein the alkyl group is an alpha-branched C₂-C₅ alkyl group
a C₃-C₈ cycloalkyl group or C₅-C₈ cycloalkenyl group, either of which may optionally be substituted by one or more hydroxyl, or one or more C₁-C₄ alkyl groups or halo atoms
a 3 to 6 membered oxygen or sulphur containing heterocyclic ring which may be saturated, or fully or partially unsaturated and which may optionally be substituted by one or more C₁-C₄ alkyl groups, halo atoms or hydroxyl groups
phenyl which may be optionally substituted with at least one substituent selected from C₁-C₄ alkyl, C₁-C₄ alkoxy and C₁-C₄ alkylthio groups, halogen atoms, trifluoromethyl, and cyano or
R17-CH₂- where R¹⁷ is H, C₁-C₈ alkyl, C₂-C₈ alkenyl, C₂-C₈ alkynyl, alkoxyalkyl or alkylthioalkyl containing from 1 to 6 carbon atoms in each alkyl or alkoxy group wherein any of said alkyl, alkoxy, alkenyl or alkynyl groups may be substituted by one or more hydroxyl groups or by one or more halo atoms; or a C₃-C₈ cycloalkyl or C₅-C₈ cycloalkenyl either of which may be optionally substituted by one or more C₁-C₄ alkyl groups or halo atoms; or a 3 to 6 membered oxygen or sulphur containing heterocyclic ring which may be saturated or fully or partially unsaturated and which may optionally be substituted by one or more C₁-C₄ alkyl groups or halo atoms; or a group of the formula SA₁₆ wherein A₁₆ is C₁-C₈ alkyl, C₂-C₈ alkenyl, C₂-C₈ alkynyl, C₃-C₈ cycloalkyl, C₅-C₈ cycloalkenyl, phenyl or substituted phenyl wherein the substituent is C₁-C₄ alkyl, C₁-C₄ alkoxy or halo, or a 3 to 6 membered oxygen or sulphur-containing heterocyclic ring which may be saturated, or fully or partially unsaturated and which may optionally be substituted by one or more C₁-C₄ alkyl groups or halo atoms
R², R⁴, R⁵, R⁶, R⁷ and R⁹ are each independently H, OH, CH₃, C₂H₅ or OCH₃
R³═H or OH
r⁸═H, 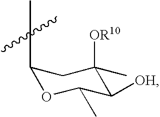
rhamnose, 2′-O-methyl rhamnose, 2′,3′-bis-O-methyl rhamnose, 2′,3′,4′-tri-O-methyl rhamnose, oleandrose, oliose, digitoxose, olivose or angolosamine;
R¹⁰═H or CH₃ or C(═O)R_{A}, where R_{A}═C1-C6 alkyl, C2-C6 alkenyl or C2-C6 alkynyl
R¹¹═H, 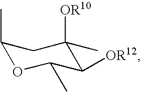
mycarose, C4-O-acyl-mycarose or glucose
R¹²═H or C(═O)R_{A}, where R_{A}═C1-C6 alkyl, C2-C6 alkenyl or c2-C6 alkynyl
R¹³═H or CH₃
R¹⁵═H or 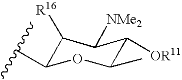
R¹⁶═H or OH
R¹⁴═H or —C(O)NR^{c}R^{d} wherein each of R^{c} and R^{d} is indpeendently H, C₁-C₁₀ alkyl, C₂-C₂₀ alkenyl, C₂-C₁₀ alkynyl, —(CH₂)ₘ(C₆-C₁₀ aryl), or —(CH₂)ₘ(5-10 membered heteroaryl), wherein m is an integer ranging from 0 to 4, and wherein each of the foregoing R^{c} and R^{d} groups, except H, may be substituted by 1 to 3 Q groups; or wherein R^{c} and R^{d} may be taken together to form a 4-7 membered saturated ring or a 5-10 membered heteroaryl ring, wherein said saturated and heteroaryl rings may include 1 or 2 heteroatoms selected from O, S and N, in addition to the nitrogen to which R^{c} and R^{d} are attached, and said saturated ring may include 1 or 2 carbon-carbon double or triple bonds, and said saturated and heteroaryl rings may be substituted by 1 to 3 Q groups; or R² and R¹⁷ taken together form a carbonate ring; each Q is independently selected from halo, cyano, nitro, trifluoromethyl, azido, —C(O)Q¹, —OC(O)Q¹, —C(O)OQ¹, —OC(O)OQ¹, —NQ²C(O)Q³, —C(O)NQ²Q³, —NQ²Q³, hydroxy, C₁-C₆ alkyl, C₁-C₆ alkoxy, —(CH₂)ₘ(C₆-C₁₀ aryl), and —(CH₂)ₘ(5-10 membered heteroaryl), wherein m is an integer ranging from 0 to 4, and wherein said aryl and heteroaryl substituents may be substituted by 1 or 2 substituents independently selected from halo, cyano, nitro, trifluoromethyl, azido, —C(O)Q¹, —C(O)OQ¹, —OC(O)OQ¹, —NQ²C(O)Q³, —C(O)NQ²Q³, —NQ²Q³, hydroxy, C₁-C₆ alkyl, and C₁-C₆ alkoxy;
each Q¹, Q² and Q³ is independently selected from H, OH, C₁-C₁₀ alkyl, C₁-C₆ alkoxy, C₂-C₁₀ alkenyl, C₂-C₁₀ alkynyl, —(CH₂)m(C₆-C₁₀ aryl),a nd —(CH₂)ₘ(5-10 membered heteroaryl), wherein m is an integer ranging from 0 to 4; with the proviso that the compound is not 5-O-dedesosaminyl-5-O-mycaminosyl erythromycin A or D or said compound is a variant of any of the above in which the —CHOR¹⁴— at C11 is replaced by a methylene group (—CH₂—), a keto group (C═O), or by a 10,11-olefinic bond;
or said compound is a variant of any of the above which differs in the oxidation state of one or more of the ketide units (i.e. selection of alternatives from the group: —CO—, —CH(OH)—, alkene —CH—, and CH₂ );
with the proviso that the compounds are not selected from the group consisting of 5-O-dedesosaminyl-5-O-mycaminosyl erythromycin A and 5-O-dedesosaminyl-5-O-mycaminosyl erythromycin D.

21. A compound according to the formula II below: 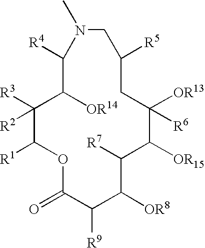
R¹ is selected from:
H, CH₃, C₂H₅
an alpha-branched C₃-C₈ group selected from alkyl, alkenyl, alkynyl, alkoxyalkyl and alkylthioalkyl groups any of which may be optionally substituted by one or more hydroxyl groups;
a C₅-C₈ cycloalkylalkyl group wherein the alkyl group is an alpha-branched C₂-C₅ alkyl group
a C₃-C₈ cycloalkyl group or C₅-C₈ cycloalkenyl group, either of which may optionally be substituted by one or more hydroxyl, or one or more C₁-C₄ alkyl groups or halo atoms
a 3 to 6 membered oxygen or sulphur containing heterocyclic ring which may be saturated, or fully or partially unsaturated and which may optionally be substituted by one or more C₁-C₄ alkyl groups, halo atoms or hydroxyl groups
phenyl which may be optionally substituted with at least one substituent selected from C₁-C₄ alkyl, C₁-C₄ alkoxy and C₁-C₄ alkylthio groups, halogen atoms, trifluoromethyl, and cyano or
R¹⁷-CH₂- where R¹⁷ is H, C₁-C₈ alkyl, C₂-C₈ alkenyl, C₂-C₈ alkynyl, alkoxyalkyl or alkylthioalkyl containing from 1 to 6 carbon atoms in each alkyl or alkoxy group wherein any of said alkyl, alkoxy, alkenyl or alkynyl groups may be substituted by one or more hydroxyl groups or by one or more halo atoms; or a C₃-C₈ cycloalkyl or C₅-C₈ cycloalkenyl either of which may be optionally substituted by one or more C₁-C₄ alkyl groups or halo atoms; or a 3 to 6 membered oxygen or sulphur containing heterocyclic ring which may be saturated or fully or partially unsaturated and which may optionally be substituted by one or more C₁-C₄ alkyl groups or halo atoms; or a group of the formula SA₁₆ wherein A₁₆ is C₁-C₈ alkyl, C₂-C₈ alkenyl, C₂-C₈ alkynyl, C₃-C₈ cycloalkyl, C₅-C₈ cycloalkenyl, phenyl or substituted phenyl wherein the substituent is C₁-C₄ alkyl, C₁-C₄ alkoxy or halo, or a 3 to 6 membered oxygen or sulphur-containing heterocyclic ring which may be saturated, or fully or partially unsaturated and which may optionally be substituted by one or more C₁-C₄ alkyl groups or halo atoms
R², R⁴, R⁵, R⁶, r⁷ and R⁹ are each independently H, OH, CH₃, C₂H₅ or OCH₃
R³═H or OH
R⁸═H, 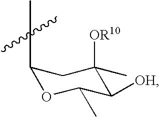
rhamnose, 2′-O-methyl rhamnose, 2′,3′-bis-O-methyl rhamnose, 2′,3′,4′-tri-O-methyl rhamnose, oleandrose, oliose, digitoxose, olivose or angolosamine;
R¹⁰═H or CH₃ or C(═O)R_{A}, where R_{A}═C1-C6 alkyl, C2-C6 alkenyl or c2-C6 alkynyl
R¹¹═H, 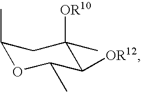
mycarose, C4-O-acyl-mycarose or glucose
R¹²═H or C(═O)R_{A}, where R_{A}═C1-C6 alkyl, C2-C6 alkenyl or C2-C6 alkynyl
R¹³═H or CH₃
R¹⁵═H or 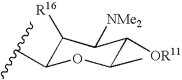
R¹⁶═H or OH
R¹⁴═H or —C(O)NR^{c}R^{d} wherein each of R^{c} and R^{d} is independently H, C₁-C₁₀ alkyl, C₂-C₂₀ alkenyl, C₂-C₁₀ alkynyl, —(CH₂)ₘ(C₆-C₁₀ aryl), or —(CH₂)ₘ(5-10 membered heteroaryl), wherein m is an integer ranging from 0 to 4, and wherein each of the foregoing R^{c} and R^{d} groups, except H, may be substituted by 1 to 3 Q groups; or wherein R^{c} and R^{d} may be taken together to form a 4-7 membered saturated ring or a 5-10 membered heteroaryl ring, wherein said saturated and heteroaryl rings may include 1 or 2 heteroatoms selected from O, S and N, in addition to the nitrogen to which R^{c} and R^{d} are attached, and said saturated ring may include 1 or 2 carbon-carbon double or triple bonds, and said saturated and heteroaryl rings may be substituted by 1 to 3 Q groups; or R² and R¹⁷ taken together form a carbonate ring; each Q is independently selected from halo, cyano, nitro, trifluoromethyl, azido, —C(O)Q¹, —OC(O)Q¹, —C(O)OQ¹, —OC(O)OQ¹, —NQ²C(O)Q³, —C(O)NQ²Q³, —NQ²Q³, hydroxy, C₁-C₆ alkyl, C₁-C₆ alkoxy, —(CH₂)ₘ(C₆-C₁₀ aryl), and —(CH₂)ₘ(5-10 membered heteroaryl), wherein m is an integer ranging from 0 to 4, and wherein said aryl and heteroaryl substituents may be substituted by 1 or 2 substituents independently selected from halo, cyano, nitro, trifluoromethyl, azido, —C(O)Q¹, —C(O)OQ¹, —OC(O)OQ¹, —NQ²C(O)Q³, —C(O)NQ²Q³, —NQ²Q³, hydroxy, C₁-C₆ alkyl, and C₁-C₆ alkoxy;
each Q¹, Q² and Q³ is independently selected from H, OH, C₁-C₁₀ alkyl, C₁-C₆ alkoxy, C₂-C₁₀ alkenyl, C₂-C₁₀ alkynyl, —(CH₂)m(C₆-C₁₀ aryl), and —(CH₂)ₘ(5-10 membered heteroaryl), wherein m is an integer ranging from 0 to 4;
or said compound is a variant of any of the above in which the —CHOR¹4— at C12 is replaced by a methylene group (—CH2—), a keto group (C═O), or by a 11,12-olefinic bond;
or said compound is a variant of any of the above which differs in the oxidation state of one or more of the ketide units (i.e. selection of alternatives from the group: —CO—, —CH(OH)—, alkene —CH—, and CH₂).

22. A compound according to claim 20, wherein: R², R⁴, R⁵, R⁶, R⁷ and R⁹ are all CH₃.

23. A compound according to claim 22, wherein
R¹¹═H or 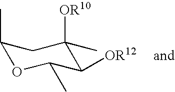
R¹⁴═H.

24. A compound according to claim 23, wherein R¹═C₂H₅ optionally substituted with a hydroxyl group.

25. A compound according to claim 24, wherein R¹²═H.

26. A compound according to claim 25, wherein R¹═C₂H₅.

27. A compound according to claim 21, wherein: R²₁ R⁴, R⁵, R⁶, R⁷ and R⁹ are all CH₃.

28. A compound according to claim 27, wherein
R¹¹═H or 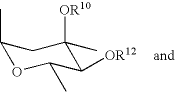
R¹⁴═H,

29. A compound according to claim 28, wherein R¹═C₂H, optionally substituted with a hydroxyl group.

30. A compound according to claim 29, wherein R¹²═H.

31. A compound according to claim 25, wherein R¹═C₂H₅.


SEQUENCE LISTING (57 sequences)

SEQ ID NO: 1 - protein, 305 aa, Streptomyces fradiae
MNDRPRRAMKGIILAGGSGTRLRPLTGTLSKQLLPVYDKPMIYYPLSVLMLAGIREIQIISSKDHLDLFRSLLGEGDRLGLSISYAEQREPRGIAEAFLIGARHIGGDDAALILGDNVFHGPGFSSVLTGTVARLDGCELFGYPVKDAHRYGVGEIDSGGRLLSLEEKPRRPRSNLAVTGLYLYTNDVVEIARTISPSARGELEITDVNKVYLEQGRARLTELGRGFAWLDMGTHDSLLQAGQYVQLLEQRQGERIACIEEIAMRMGFISAEQCYRLGQELRSSSYGSYIIDVAMRGAAADSRAQ

SEQ ID NO: 2 - protein, 303 aa, Streptomyces fradiae
MNDRPRRAMKGIILAGGSGTRLRPLTGTLSKQLLPVYDKPMIYYPLSVLMLAGIREIQIISSKDHLDLFRSLLGEGDRLGLSISYAEQREPRGIAEAFLIGARHIGGDDAALILGDNVFHGPGFSSVLTGTVARLDGCELFGYPVKDAHRYGVGEIDSGGRLLSLEEKPRRPLEPGRHRLYLYTNDVVEIARTISPSARGELEITDVNKVYLEQGRAAHGAGAVVAWLDMGTHDSLLQAGQYVQLLEQRQGERIACIEEIAMRMGFISAEQCYRLGQELRSSSYGSYIIDVAMRGAAADSRAQ

SEQ ID NO: 3 - protein, 333 aa, Streptomyces fradiae
MRVLVTGGAGFIGSHFTGQLLTGAYPDLGATRTVVLDKLTYAGNPANLEHVAGHPDLEFVRGDIADQALVRRLMEGVGLVVHFAAESHVDRSIESSEAFVRTNVEGTRVLLQAAVDAGVGRFVHISTDEVYGSIAEGSWPEDHPLAPNSPYAATKAASDLLALAYHRTYGLDVRVTRCSNNYGPRQYPEKAVPLFTTNLLDGLPVPLYGDGGNTREWLHVDDHCRGVALVAAGGRPGVIYNIGGGTELTNAELTDRILELCGADRSAVRRVADRPGHDRRYSVDTTKIREELGYAPRTGITEGLAGTVAWYRDNRAWWEPLKRSPGGRELERA

SEQ ID NO: 4 - protein, 333 aa, Streptomyces fradiae
MRVLVTGGAGFIGSHFTGQLLTGAYPDLGATRTVVLDKLTYAGNPANLEHVAGHPDLEFVRGDIADHGWWRRLMEGVGLVVHFAAESHVDRSIESSEAFVRTNVEGTRVLLQAAVDAGVGRFVHISTDEVYGSIAEGSWPEDHPVAPNSPYAATKAASDLLALAYHRTYGLDVRVTRCSNNYGPRQYPEKAVPLFTTNLLDGLPVPLYGDGGNTREWLHVDDHCRGVALVGAGGRPGVIYNIGGGTELTNAELTDRILELCGADRSALRRVADRPGHDRRYSVDTTKIREELGYAPRTGITEGLAGTVAWYRDNRAWWEPLKRSPGGRELERA

SEQ ID NO: 5 - DNA, 2160 bp, Streptomyces eurythermus
ggcatgccttcggggtgtgcggcggcgcctcagagcgtggccagtacctcgtgcagggccgcgatcaccttgtcctgtacgtcgggcgcgagccccgggtacatcggcagcgagaagatctcgtccgccagccgctccgtcaccggcagcgagcccttggcgtaccccaggtgcgcgaagcccgtcatggtgtgcacgggccacgggtaactgatgttgagcgagatcccgtacgacttgagcgcctcgatgatgtcgtcccggcgcgggtggcggacgacgtacacgtaatacacgtggtcgttgccctcggtgacggacggcagcaccaggccgccggggcccgtcaggttcgcgagtccttcggcgtaacgccgggcgaccgcgcgccggccctcgatgtagcggtcgaggcgggtgagcttgcggcgcaggatctccgcctgcacctcgtcgagccggctgttgtggccgggcgtctgcacgacgtagtacacgtcctccatgccgtagtagcgcagccggcgcagcgcacggtcgacgtccgcgtcgtcggtcagcacggccccgccgtcgccgtacgcaccgaggaccttcgtcgggtagaacgagaaggcggcggcgtcgcccagcgtgccggccagctcgccgtggtggcgggcaccgtgcgcctgggcgcagtcctccagcaccaccaggccgtgctgctcggccagggcgcgcaagggcgccatgtcgacgcactgcccgtacaggtgcaccggcagcagggccttcgtgcgcggggtgatgacgtccgcgacctggtcggtgtccatgaggtggtcctcggcgcggacgtcgacgaagacgggcgtggcaccggtgccgtcgatggccaccaccgtcggcgcggccgtgttggagacggtgacgacctcgtcccccgggcccaccccgagcgcctgcagacccagcttgacggcgttggtgccgttgtcgacaccgccgcagtggcgcaggccgtggtagtccgcgaactccttctcgaacccgtccacgctggggccgaggaccaactgcccggaggcgaagacggtctcgacggcgtcgaggaggtccgcgcgttcgttctggtattccgccaggtagtcccagacgtaggtagtcacggagagctcaacctccagagtgtttcgatggggtggtgggaagccggtgcgcgcggaccaggtcgtgccagcagtcgcggaccgactcccgcagcgaacggcgcggtgcccagcccagcagggcgcgcgccgcgccggtgtcgacccgcagccagtcctcccggtgcccgggagcccggcccggagccgggcgctccaccacccgcgccggaatgccgctcgcctcgatgaacaggccgaccaggtcgcggacggcgaccgcctcgccccgcccgatgccgacggcgaccgggacggccggtgcgcgggcggcggccacgacggcgtcggccacgtcccgcacatcgacgtagtcccggtgcgcgcgcagccgggacagttccacgacggcctccgcacccgtcccggcggccgccagcagccgctcggcgacctggcccagcagactgatccgcggggtgccggggcccgacacgttggacacccgtagcaccacaccgtcgacccacccgcccgaggtgccccgcagcaccgcctcgctggcggcgagcttgctcctgccgtacgccgtgtccgggcgcggtacggcgtcggcgcccaccgaaccgccgggcgtcaccgggccgtactccagtaccgagccgaggtggaccagccgcggccgcgcggacatcagcgccagcgcctccagcaggcgcagcgtgggcaccgcggtggcggaccacatctgctcgtcggtacggccccagatgcttccgacggagttgacgatcgtgtccggacgctccgcgtccagggcggcggccagcgccgcgggatccgtaccggccaggtccagggtgacgcagcggtacggcatcggctcctcgggcgggcggcggcccaccaccaccacgtcacggccccgcgcggcgaacgccgcgcacacatgccggccgacgtacccggcgccgcccaggaccacgacgctgccactgccactgccgcgcggcatcggatcgttcaccat

SEQ ID NO: 6 - DNA, 4461 bp, Streptomyces eurythermus
cgtcagtacagcgtgtgggcacacgccaccagggtgcgcagctcgatgttgaggtagttgccgtgcgccagcagcccggtgagctgaccgagcgacagccaggcgaagtcgtccggtgcgtcctccgggaagtcgtgcgggacctccacgatcacgtagcggttctgggcgtggaagaagcgcccgccctcctcggactggacggcgtcgtagcgcacgtcctgaggcggcgcggacagcacgtcctccaggtacggcgggccgggcagcccccgcggaccggtgtgctcctgtggccggcactggaccgtgggggccagctcggcgacgttcaggtgcccgacgtccacccgtgcccgcacgagcgcgtgcagcacgccgtcgacggacttgaccagcagcgccatcagacccggcagccgcggctcgatgagcggctgcgtccaggaggtgacctcccggctgctggcgctgacctcggcggccatgacccggaagtgccgcccgctctcgtgggcgatctcgtgcggcgtgcggtaccagccgtccgccgtcaccgtatcgagcggcacccggttctgcaccagctcccgcagggcgcgcacacccgtgaaccacgtcaggacctcggccgtcgtgtgccgcgccgcacccggcgagccgaagaaggagcgcagcacgggggacggggcggacgcgtcggcgtccgccgtgggcaggcaggcgaggatggaccgggcgtccatgttgaccacgttgtccagcatcagcagccggcggagctgccccagcgtcagccagcggaagtcctccccgatgtcgaggtcgtcgtccgccgccaactcgacgatcatgttccggttgcgtttggccaggaaccagtccgcctgttcggactggatcgagtcgaccaggacacgcgcccgtcgcggccccatgaacaggtccagatagcggatgtcgcgcccccggtgcaccccggtgaagttgctccgggtggcctgcacggtcggcgacacctgaagaacgttgacgttcccgggctccatcttggcctgcatcaggaagtgcagcaccccgtcgatctcccgcgccacgatcccgagcagccccacctccggctgcacgatgatgggctgcgtccagccccgctcgggcagccggtccgtacggacgtgcagcccctccacggagaagaaacggcccgacgcgtggtgcaggtttcccgtacccgggtggaagctccagccgcgcagctccgcgaagggaacgcgggacacgtcgaagcgccccgcccgcaggcgttcggccagccagccggagatgccgtcgaacggcgtgaccgcactgtccgcggtgcgtgccgacaccagcacccgccgcgccgtgtccaccgggtcaccgggccggaccgcgtccgcacggcgccgcgcggcgccgtgcggggcgggggcggatcgcggcggtacgggttcgcgggcggtgtccgcggcggtgcgcggcgggacggggccggtgctcgtgtccgcggcggtacgcggtgggacggtcccggtggccgtgtccgcggtggccgtgccggcgagggcgtcgccgatggtccggcacacctcgtccatccggtcgttcagatagaagtgaccgccggcgaaggtgtgcagggcgaaggggcccgtggtcagctcccgccaggccctcgcctcctccagcgggacatcgggatcacggtcaccggtgagcaccgtgaccggacagtccagcgcaccgccgggcacatacgcgtacgtgcccgccgcccggtagtcgttgcggatcgccggcagggccagccgcagcagctcctcgtcctggaggacggcgtcctcggtgccctgaagcgtggcgatctccgcgatcagcgcgtcgtcgtcgaggaggtgggcgacgtcccgccggcgcaccgtcggcgcacggcggcccgacaccagcagatggacgggggaggcctgcccggaaccgcgcagccggcgcgcgacctcgaacgccaccgtggcacccatgctgtgcccgaacagcgcgagcggacggtcggcccagcgcaggatctccggcaccacctggtccaccaggcccgatatggacgggatgaacggctcgtgccggcggtcctggcggcccgggtactgcaccgccagcgcctccacggtctcgtccagtccgcgtgccagggcggcgaaggaggtcgcggcgccaccggcgtgcgggaagcagaccagacgcagttccggatcccgcaccgggcggtaacggcggacccacagaccctcgtccgggtgtccggccggcgacggggctcccggaacgggtggtgcggaaggggtgctcacggcggatccagctcctcgcggtcggggggaccgctgtcggggacggcacgtcgggtgcggacgtcgggtacgggcgtcggggcgtgacggggagggacggggcggtcggtcagtcggtgcgccgggcctcctgcgcggccttcttcagcggttcccaccacgcgcggttctccgcgtaccagcgcaccgtgtccgccaggcccgtcgtgaagtccgtacgcggggcatagcccagctcgcccgtgatcttgccgatgtccagcgcgtaccgcaggtcgtgccccggccggtcggcgacgtggcgcaccgacgaggcgtcggcaccgcacagcccgagcagccgcttcgtcagctcccggttggtcagctccgtcccgccaccgatgtggtagacctcgcccgggcgcccgcgggtcgccaccaggctgatcccgcggcagtggtcgtccacgtgcagccagtcccggctgttgccgccgtcgctgtacagcggcaccgtcagaccgtccaacaggttcgtggcgaagagcgggacgaccttctcggggtgctggtacgggccgtagttgttggagcaccgggtgacgacgaccggcaggccgtacgtccggtggtaggccagcgccaggaggtccgacgccgccttcgaggcggcgtacggggagttcggcgccagcggctgctcctcgcgccacgacccctcggcgatcgagccgtacacctcgtccgtggagacgtggacgaaccggccggcccccgcctccaccgcggcctgcaagaggacttgcgtcccccgtacgttcgtctcgacgaacgccgacgcgtcggcgatggagcggtccacgtgcgactccgccgcgaagtggaccacgacgtccgccccccgcacgacccgggacatcacctccgcgtcccggatgtcggcgtgcacgaactccagcgacggatggtccgcgaccgggtccaggttggcgaggttcccggcataggtcagcttgtcgaccaccaccgtccgcgccccggccaggtccggatacgccccggccagcagttgtctgacgaagtgcgagccgatgaagcccgcacctccggtgaccagcagccgcatgggagcacagacctttcttccagggacgggaaacggggaggcggacggggacggaggcgagggcggtggctatgcggccggtccggacatgagggtctccgccacgtccatcaagtaccggccgtagctggagctctcgagttcacggccgagctcgtggcactgccgcgcgctgatgtaccccatccgcagggcgatctcctcgacgcaggagatccgcacgccctgccgctgctccaggagctggacgtactgccccgcttgcagcagcgagctgtgcgtgcccatgtccagccaggcgaacccgcgccccagttccgtcatacgggcgcggccctgctccaggtacaccttgttgacgtcggtgatctccagctcgccccgcggcgacggtgtcagccgccgggcgatgtccaccacgccgttgtcgtagaagtacagccccgtcaccgcgagatgggagcggggcttctccggcttctcctccagggacaccagccggccttccgcgtcgacctcgccgacgccgtagcgccgggggtccttcaccgggtagccgaacagctcgcagccgtccagccgcgccgcggtggaggccagcacggaggagaaccccggaccgtggaagacgttgtcccccaggatgagggcgaccgggtcgtccccgatgtgctcctcgccgatgaggaacgcctcggcgatgccccggggctcctcctgctcggcgtagccgacactgatcccgatgcggctgccgtcgcccagcagcgaacggaacatctccaagtgcgtcttcgacgtgatgatctggatgtcccggatccccgccagcatgagcaccgacagcgggtagtagatcatgggcttgtcgtagaccggcagcaactgcttggacagtgccccggtcagggggcgcaggcgcgtgccgctgccgcccgccaggatgatgcccttcatgggccgccggtccgccgtcgtcttcgtcat

SEQ ID NO: 7 - DNA, 3375 bp, Streptomyces eurythermus
gtgagccccgcacccgccaccgaggacccggccgccgccgggcgccgcctgcaactgacccgcgcagcccagtggttcgcgggaacccaggacgacccgtacgcgctcgtcctgcgcgccgaggccaccgacccggccccgtacgaggagcggatccgggcccacgggccgctcttccgcagcgacctgctcgacacctgggtcacggcgagcagggccgtcgccgacgaagtgatcacctcacccgccttcgacgggctcacggccgacgggcggcgccccggcgcgcgggaactgccgctgtccggcaccgcgctcgacgcggaccgcgccacatgcgcacggttcggggccctcaccgcctggggcgggccgctgctgccggcgccgcacgagcgggcgctgcgcgagtccgccgaacggcgggcccacacactcctcgacggggcggaggccgccctggccgccgacggcaccgtcgacctcgtcgacgcgtacgcccgcaggctccccgcgctggtcctccgcgaacagctcggcgtgccggaggaggcggcgaccgccttcgaggacgcgctggccggctgccgccgcaccctggacggcgccctgtgcccgcaactcctcccggacgccgtggcgggggtgcgcgcggaagccgcgctgaccgccgtgctggcctccgccctgcgcgggactccggccggccgggcccccgacgccgtcgccgccgcccgcaccctggccgtcgcggccgccgagcccgcagccaccctcgtcggcaacgccgtacaggagctgctggcgcgtcccgcgcagtgggcggagctcgtacgcgacccgcgcctcgcggccgccgcggtgaccgaaacgctgcgtgtcgccccgcccgtccgcctggagcggcgggtcgcccgcgaggacacggacatcgccgggcagcgcctccccgccggggggagcgtcgtgatcctcgtcgccgccgtcaaccgcgcgcccgtatccgcgggaagcgacgcctccaccaccgtcccgcacgccggcggccggccccgtacctccgccccctccgtcccctcagcccccttcgacctcacacggcccgtggccgcgcccgggccgttcgggctccccggcgacctgcacttccgcctcggcgggcccctggtcggaacggtcgccgaagccgcgctcggtgcgctggccgcacggctccccggtctgcgcgccgccgggccggccgtgcggcgccgccgctcaccggtgctgcacggacacgcccgcctccccgtcgccgtcgcccggacggcccgtgacctgcccgccaccgcaccgcggaactgaggagggagtgccccgatgcgtatcctgctgacgtcgttcgcgcacaacacgcactactacaacctggtccccctcggctgggcgctgcgcgccgccgggcacgacgtacgggtcgccagccagccctcgctgaccggcaccatcaccggctccgggctgaccgccgtccccgtgggcgacgacacggccatcgtcgagctgatcaccgagatcggcgacgacctcgtcctctaccagcagggcatggacttcgtggacacccgcgacgagccgctgtcctgggaacacgccctcggacagcagacgatcatgtcggccatgtgcttctcgccgctgaacggcgacagcaccatcgacgacatggtggcgctggcccgttcctggaaaccggacctcgtcctgtgggagcccttcacctacgcgggacccgtcgccgcgcacgcctgcggcgccgcccacgcccggctgctgtggggtcccgacgtggtcctcaacgcacggcggcagttcacccggctgctcgccgagcgccccgtcgaacagcgcgaggacccggtcggcgaatggctcacgtggacgctggagcgccacggcctcgccgccgacgcggacacgatcgaggaactgttcgccgggcagtggacgatcgaccccagcgccgggagcctgcggctgccggtcgacggcgaggtcgtgcccatgcgcttcgtgccgtacaacggcgcctcggtcgtccccgcctggctctccgagccgcctgcccggccccgggtctgcgtcaccctcggcgtctccacccgggagacctacggcacggacggcgtcccgttccacgaactgctggccggactggccgacgtggacgccgagatcgtcgccaccctcgacgcggggcagctcccggacgccgccggtctgcccggcaatgtgcgcgtcgtcgacttcgtgccgctggacgccctgctgccgagctgcgccgcgatcgtccaccacggaggcgcgggaacctgtttcacggccaccgtgcacggcgtcccgcagatcgtcgtggcctccctctgggacgcgccgctgaaggcgcaccaactcgccgaggcgggcgccgggatcgccctggaccccggggaactgggcgtggacaccctgcgcggcgccgtcgtgcgggtgctggagagccgcgagatggccgtggcggcgcgtcgcctcgccgacgagatgctcgccgcccccaccccggccgcgctcgtcccccgcctcgaacgcctcaccgccgcgcaccgccgcgcctgatcccgccaaggagcccccatgaacctcgaatacagcggcgacatcgcccggttgtacgacctggtccaccagggaaagggcaaggactaccgggcggaggccgaggagctggccgcgcttgtcacccagcgccgccccggggcccgctccctcctcgacgtggcctgcggaacggggatgcacctgcggcacctcggcgacctcttcgaggaggtggccggggtggagatgtcccccgacatgctggccatcgcgcagcggcgcaacccggaggccggcatccaccggggggacatgcgggacttcgccctcggccgccgcttcgacgccgtgatctgcatgttcagttccatcgggcacatgcgcgaccagcgggaactggacgcggcgatcggccggttcgccgcgcacctgccgtccggcggggtcgtgatcgtcgatccctggtggttcccggagacgttcacaccggggtacgtcggcgcgagcctcgtcgaggccgagggccgcaccatcgcgcgcttctcccactccgcgctcgaggacggcgcgacccggatcgatgtggactacctcgtcggcgtgccgggggagggggtgcggcacttgaaggagacccatcggatcacgcttttcgggcgtgcgcagtacgaggcggccttcaccgcggcggggatgtccgtcgagtacctcccgcacgccgccaccgaccgcggactcttcgtcggcgtccaggcctga

SEQ ID NO: 8 - protein, 295 aa, Streptomyces eurythermus
MKGIILAGGSGTRLRPLTGALSKQLLPVYDKPMIYYPLSVLMLAGIRDIQIITSKTHLEMFRSLLGDGSRIGISVGYAEQEEPRGIAEAFLIGEEHIGDDPVALILGDNVFHGPGFSSVLASTAARLDGCELFGYPVKDPRRYGVGEVDAEGRLVSLEEKPEKPRSHLAVTGLYFYDNGVVDIARRLTPSPRGELEITDVNKVYLEQGRARMTELGRGFAWLDMGTHSSLLQAGQYVQLLEQRQGVRISCVEEIALRMGYISARQCHELGRELESSSYGRYLMDVAETLMSGPAA

SEQ ID NO: 9 - protein, 332 aa, Streptomyces eurythermus
MRLLVTGGAGFIGSHFVRQLLAGAYPDLAGARTVVVDKLTYAGNLANLDPVADHPSLEFVHADIRDAEVMSRVVRGADVVVHFAAESHVDRSIADASAFVETNVRGTQVLLQAAVEAGAGRFVHVSTDEVYGSIAEGSWREEQPLAPNSPYAASKAASDLLALAYHRTYGLPVVVTRCSNNYGPYQHPEKVVPLFATNLLDGLTVPLYSDGGNSRDWLHVDDHCRGISLVATRGRPGEVYHIGGGTELTNRELTKRLLGLCGADASSVRHVADRPGHDLRYALDIGKITGELGYAPRTDFTTGLADTVRWYAENRAWWEPLKKAAQEARRTD

SEQ ID NO: 10 - protein, 787 aa, Streptomyces eurythermus
VSTPSAPPVPGAPSPAGHPDEGLWVRRYRPVRDPELRLVCFPHAGGAATSFAALARGLDETVEALAVQYPGRQDRRHEPFIPSISGLVDQVVPEILRWADRPLALFGHSMGATVAFEVARRLRGSGQASPVHLLVSGRRAPTVRRRDVAHLLDDDALIAEIATLQGTEDAVLQDEELLRLALPAIRNDYRAAGTYAYVPGGALDCPVTVLTGDRDPDVPLEEARAWRELTTGPFALHTFAGGHFYLNDRMDEVCRTIGDALAGTATADTATGTVPPRTAADTSTGPVPPRTAADTAREPVPPRSAPAPHGAARRRADAVRPGDPVDTARRVLVSARTADSAVTPFDGISGWLAERLRAGRFDVSRVPFAELRGWSFHPGTGNLHHASGRFFSVEGLHVRTDRLPERGWTQPIIVQPEVGLLGIVAREIDGVLHFLMQAKMEPGNVNVLQVSPTVQATRSNFTGVHRGRDIRYLDLFMGPRRARVLVDSIQSEQADWFLAKRNRNMIVELAADDDLDIGEDFRWLTLGQLRRLLMLDNVVNMDARSILACLPTADADASAPSPVLRSFFGSPGAARHTTAEVLTWFTGVRALRELVQNRVPLDTVTADGWYRTPHEIAHESGRHFRVMAAEVSASSREVTSWTQPLIEPRLPGLMALLVKSVDGVLHALVRARVDVGHLNVAELAPTVQCRPQEHTGPRGLPGPPYLEDVLSAPPQDVRYDAVQSEEGGRFFHAQNRYVIVEVPHDFPEDAPDDFAWLSLGQLTGLLAHGNYLNIELRTLVACAHTLY

SEQ ID NO: 11 - protein, 333 aa, Streptomyces eurythermus
MVNDPMPRGSGSGSVVVLGGAGYVGRHVCAAFAARGRDVVVVGRRPPEEPMPYRCVTLDLAGTDPAALAAALDAERPDTIVNSVGSIWGRTDEQMWSATAVPTLRLLEALALMSARPRLVHLGSVLEYGPVTPGGSVGADAVPRPDTAYGRSKLAASEAVLRGTSGGWVDGVVLRVSNVSGPGTPRISLLGQVAERLLAAAGTGAEAVVELSRLRAHRDYVDVRDVADAVVAAARAPAVPVAVGIGRGEAVAVRDLVGLFIEASGIPARVVERPAPGRAPGHREDWLRVDTGAARALLGWAPRRSLRESVRDCWHDLVRAHRLPTTPSKHSGG

SEQ ID NO: 12 - protein, 373 aa, Streptomyces eurythermus
VTTYVWDYLAEYQNERADLLDAVETVFASGQLVLGPSVDGFEKEFADYHGLRHCGGVDNGTNAVKLGLQALGVGPGDEVVTVSNTAAPTVVAIDGTGATPVFVDVRAEDHLMDTDQVADVITPRTKALLPVHLYGQCVDMAPLRALAEQHGLVVLEDCAQAHGARHHGELAGTLGDAAAFSFYPTKVLGAYGDGGAVLTDDADVDRALRRLRYYGMEDVYYVVQTPGHNSRLDEVQAEILRRKLTRLDRYIEGRRAVARRYAEGLANLTGPGGLVLPSVTEGNDHVYYVYVVRHPRRDDIIEALKSYGISLNISYPWPVHTMTGFAHLGYAKGSLPVTERLADEIFSLPMYPGLAPDVQDKVIAALHEVLATL

SEQ ID NO: 13 - protein, 447 aa, Streptomyces eurythermus
VSPAPATEDPAAAGRRLQLTRAAQWFAGTQDDPYALVLRAEATDPAPYEERIRAHGPLFRSDLLDTWVTASRAVADEVITSPAFDGLTADGRRPGARELPLSGTALDADRATCARFGALTAWGGPLLPAPHERALRESAERRAHTLLDGAEAALAADGTVDLVDAYARRLPALVLREQLGVPEEAATAFEDALAGCRRTLDGALCPQLLPDAVAGVRAEAALTAVLASALRGTPAGRAPDAVAAARTLAVAAAEPAATLVGNAVQELLARPAQWAELVRDPRLAAAAVTETLRVAPPVRLERRVAREDTDIAGQRLPAGGSVVILVAAVNRAPVSAGSDASTTVPHAGGRPRTSAPSVPSAPFDLTRPVAAPGPFGLPGDLHFRLGGPLVGTVAEAALGALAARLPGLRAAGPAVRRRRSPVLHGHARLPVAVARTARDLPATAPRN

SEQ ID NO: 14 - protein, 424 aa, Streptomyces eurythermus
MRILLTSFAHNTHYYNLVPLGWALRAAGHDVRVASQPSLTGTITGSGLTAVPVGDDTAIVELITEIGDDLVLYQQGMDFVDTRDEPLSWEHALGQQTIMSAMCFSPLNGDSTIDDMVALARSWKPDLVLWEPFTYAGPVAAHACGAAHARLLWGPDVVLNARRQFTRLLAERPVEQREDPVGEWLTWTLERHGLAADADTIEELFAGQWTIDPSAGSLRLPVDGEVVPMRFVPYNGASVVPAWLSEPPARPRVCVTLGVSTRETYGTDGVPFHELLAGLADVDAEIVATLDAGQLPDAAGLPGNVRVVDFVPLDALLPSCAAIVHHGGAGTCFTATVHGVPQIVVASLWDAPLKAHQLAEAGAGIALDPGELGVDTLRGAVVRVLESREMAVAARRLADEMLAAPTPAALVPRLERLTAAHRRA

SEQ ID NO: 15 - protein, 240 aa, Streptomyces eurythermus
MNLEYSGDIARLYDLVHQGKGKDYRAEAEELAALVTQRRPGARSLLDVACGTGMHLRHLGDLFEEVAGVEMSPDMLAIAQRRNPEAGIHRGDMRDFALGRRFDAVICMFSSIGHMRDQRELDAAIGRFAAHLPSGGVVIVDPWWFPETFTPGYVGASLVEAEGRTIARFSHSALEDGATRIDVDYLVGVPGEGVRHLKETHRITLFGRAQYEAAFTAAGMSVEYLPHAATDRGLFVGVQA

SEQ ID NO: 16 - DNA, 72 bp, Artificial
  primer
ggggaattcagatctggtctagaggtcagccggcgtggcggcgcgtgagttcctccagtcgcgggacgatct

SEQ ID NO: 17 - DNA, 38 bp, Artificial
  Primer
gggcatatgaacgaccgtccccgccgcgccatgaaggg

SEQ ID NO: 18 - DNA, 50 bp, Artificial
  primer
cccctctagaggtcactgtgcccggctgtcggcggcggccccgcgcatgg

SEQ ID NO: 19 - DNA, 52 bp, Artificial
  primer
cccctctagaggtcatgcgcgctccagttccctgccgcccggggaccgcttg

SEQ ID NO: 20 - DNA, 81 bp, Artificial
  primer
gggtctagatcgattaattaaggaggacattcatgcgcgtcctggtgaccggaggtgcgggcttcatcggctcgcacttca

SEQ ID NO: 21 - DNA, 40 bp, Artificial
  primer
gggcatatgtacgagggcgggttcgccgagctttacgacc

SEQ ID NO: 22 - DNA, 40 bp, Artificial
  primer
ggggtctagaggtcatccgcgcacaccgacgaacaacccg

SEQ ID NO: 23 - DNA, 38 bp, Artificial
  primer
gggcatatggcggcgagcactacgacggaggggaatgt

SEQ ID NO: 24 - DNA, 38 bp, Artificial
  primer
gggtctagaggtcacgggtggctcctgccggccctcag

SEQ ID NO: 25 - DNA, 22 bp, Artificial
  primer
catcgtcaaggagttcgacggt

SEQ ID NO: 26 - DNA, 21 bp, Artificial
  primer
gccagctcggcgacgtccatc

SEQ ID NO: 27 - DNA, 35 bp, Artificial
  primer
gggcatatgagccccgcacccgccaccgaggaccc

SEQ ID NO: 28 - DNA, 42 bp, Artificial
  primer
ggtctagaggtcagttccgcggtgcggtggcgggcaggtcac

SEQ ID NO: 29 - DNA, 41 bp, Artificial
  primer
gggcatatgcgtatcctgctgacgtcgttcgcgcacaacac

SEQ ID NO: 30 - DNA, 44 bp, Artificial
  primer
ggtctagaggtcaggcgcggcggtgcgcggcggtgaggcgttcg

SEQ ID NO: 31 - DNA, 39 bp, Artificial
  primer
ggagatctggcgcggcggtgcgcggcggtgaggcgttcg

SEQ ID NO: 32 - DNA, 42 bp, Artificial
  primer
gggcatatgaacctcgaatacagcggcgacatcgcccggttg

SEQ ID NO: 33 - DNA, 44 bp, Artificial
  primer
ggtctagaggtcaggcctggacgccgacgaagagtccgcggtcg

SEQ ID NO: 34 - DNA, 37 bp, Artificial
  primer
gggcatatgactacctacgtctgggactacctggcgg

SEQ ID NO: 35 - DNA, 40 bp, Artificial
  primer
ggtctagaggtcagagcgtggccagtacctcgtgcagggc

SEQ ID NO: 36 - DNA, 41 bp, Artificial
  primer
gggcatatggtgaacgatccgatgccgcgcggcagtggcag

SEQ ID NO: 37 - DNA, 43 bp, Artificial
  primer
ggtctagaggtcaacctccagagtgtttcgatggggtggtggg

SEQ ID NO: 38 - DNA, 39 bp, Artificial
  primer
gggcatatgaagggcatcatcctggcgggcggcagcggc

SEQ ID NO: 39 - DNA, 46 bp, Artificial
  primer
ggtctagaggtcatgcggccggtccggacatgagggtctccgccac

SEQ ID NO: 40 - DNA, 36 bp, Artificial
  primer
gggcatatgcggctgctggtcaccggaggtgcgggc

SEQ ID NO: 41 - DNA, 36 bp, Artificial
  primer
ggtctagaggtcagtcggtgcgccgggcctcctgcg

SEQ ID NO: 42 - DNA, 40 bp, Artificial
  primer
gggcatatgtgtcctccttaattaatcgatgcgttcgtcc

SEQ ID NO: 43 - DNA, 51 bp, Artificial
  primer
ggagatctggtctagatcgtgttcccctccctgcctcgtggtccctcacgc

SEQ ID NO: 44 - DNA, 36 bp, Artificial
  primer
gggcatatgagcaccccttccgcaccacccgttccg

SEQ ID NO: 45 - DNA, 40 bp, Artificial
  primer
ggtctagaggtcagtacagcgtgtgggcacacgccaccag

SEQ ID NO: 46 - DNA, 37 bp, Artificial
  primer
gggcatatgagcagttctgtcgaagctgaggcaagtg

SEQ ID NO: 47 - DNA, 41 bp, Artificial
  primer
ggtctagaggtcatcgccccaacgcccacaagctatgcagg

SEQ ID NO: 48 - DNA, 33 bp, Artificial
  primer
cccatatgaccggagttcgaggtacgcggcttg

SEQ ID NO: 49 - DNA, 33 bp, Artificial
  primer
gatactagtccgccgaccgcacgtcgctgagcc

SEQ ID NO: 50 - DNA, 38 bp, Artificial
  primer
tgcactagtggccgggcgctcgacgtcatcgtcgacat

SEQ ID NO: 51 - DNA, 36 bp, Artificial
  primer
tcgatatcgtgtcctgcggtttcacctgcaacgctg

SEQ ID NO: 52 - DNA, 36 bp, Artificial
  primer
ggtctagactacgccgactgcctcggcgaggagccc

SEQ ID NO: 53 - DNA, 36 bp, Artificial
  primer
ggcatatgttcgccgacgtggaaacgacctgctgcg

SEQ ID NO: 54 - DNA, 35 bp, Artificial
  primer
ggaattcggccaggacgcgtggctggtcaccggct

SEQ ID NO: 55 - DNA, 42 bp, Artificial
  primer
ggtctagaaagagcgtgagcaggctcttctacagccaggtca

SEQ ID NO: 56 - DNA, 38 bp, Artificial
  primer
ggcatgcaggaaggagagaaccacgatgaccaccgacg

SEQ ID NO: 57 - DNA, 41 bp, Artificial
  primer
ggtctagacaccagccgtatcctttctcggttcctcttgtg